(19) United States
(12) Patent Application Publication
(10) Pub. No.: US 2024/0158467 A1
(43) Pub. Date: May 16, 2024

(54) MATERIALS AND METHODS FOR TREATING CANCER

(71) Applicants: HUMANIGEN, INC., Short Hills, NJ (US); MAYO FOUNDATION FOR MEDICAL EDUCATION AND RESEARCH, Rochester, MN (US)

(72) Inventors: Cameron DURRANT, Bella Collina, FL (US); Dale CHAPPELL, Dolores, CO (US); Saad J. KENDERIAN, Rochester, MN (US); Michelle J. COX, Rochester, MN (US); Reona SAKEMURA, Rochester, MN (US)

(73) Assignees: HUMANIGEN, INC., Short Hills, NJ (US); MAYO FOUNDATION FOR MEDICAL EDUCATION AND RESEARCH, Rochester, MN (US)

(21) Appl. No.: 18/283,196

(22) PCT Filed: Mar. 15, 2022
(86) PCT No.: PCT/US22/20459

§ 371 Date: Sep. 20, 2023

Related U.S. Application Data
(63) Continuation of application No. 17/209,160, filed on Mar. 22, 2021.
Application No. 17/209,160 is a continuation-in-part of application No. PCT/US19/59275, filed on Oct. 31, 2019.
(60) Provisional application No. 62/753,485, filed on Oct. 31, 2018.

(51) Int. Cl. C07K 14/705; A61K 31/7105; A61K 35/17; A61K 38/17; A61K 38/46; A61K 39/395; A61P 35/00

(52) U.S. Cl. CPC C07K 14/70596; A61K 31/7105; A61K 35/17; A61K 38/1774; A61K 38/465; A61K 39/3955; A61P 35/00; C07K 14/70578; A61K 48/00

(57) ABSTRACT

This document provides methods and materials involved in treating cancer. For example, chimeric antigen receptor T cells having reduced levels of GM-CSF are provided. Also provided as methods for making and using chimeric antigen receptor T cells having reduced levels of GM-CSF.

### CROSS-REFERENCE TO RELATED APPLICATIONS

This application claims priority to U.S. application Ser. No. 17/209,160, filed Mar. 22, 2021, which is a continuation-in-part application of PCT International Application No. PCT/US19/59275, filed Oct. 31, 2019, which claims priority to U.S. Provisional Application No. 62/753,485, filed Oct. 31, 2018, which are hereby incorporated by reference.

### SEQUENCE LISTING INCORPORATION

The “.txt” Sequence Listing filed with this application by EFS and which is entitled P-588784-US2-SQL-06JUN21_ST25.txt, is 8.07 kilobytes in size and which was created on Jun. 6, 2021, is hereby incorporated by reference.

### BACKGROUND

### 1. Technical Field

This document relates to methods and materials involved in treating cancer. For example, this document provides methods and materials for using chimeric antigen receptor T cells having reduced expression levels of one or more cytokines (e.g., GM-CSF) in an adoptive cell therapy (e.g., a chimeric antigen receptor T cell therapy) to treat a mammal (e.g., a human) having cancer.

### 2. Background Information

Unprecedented results from pivotal trials evaluating the safety and efficacy of CD19 directed chimeric antigen receptor T cells (CART19) have led to the recent FDA approval of CART19 (Tisagenlecleucel) for relapsed refractory acute lymphoblastic leukemia (ALL) and CART19 (Axi-Cel) for the treatment of diffuse large B cell lymphoma (DLBCL). The application of CART cell therapy is associated with toxicities resulting in cytokine release syndrome (CRS) and neurotoxicity. Additionally, the efficacy of CART cell therapy is limited to only 40% durable remissions in lymphoma and 50-60% durable remissions in acute leukemia. Thus, a critical need exists for compositions and methods for preventing and treating immunotherapy-related toxicity that occurs during and/or after treatment of cancer with immunotherapeutic methods, such as CAR-T cell therapy.

### SUMMARY OF THE INVENTION

This document provides methods and materials for generating T cells (e.g., chimeric antigen receptor (CAR) T cells (CARTs)) having a reduced expression level of one or more cytokine (e.g., GM-CSF) polypeptides. For example, a T cell (e.g., a CART) can be engineered to have reduced GM-CSF polypeptide expression (e.g., for use in adoptive cell therapy). In some cases, a T cell (e.g., a CART) can be engineered to knock out (KO) a nucleic acid encoding one or more cytokine polypeptides (e.g., a GM-CSF polypeptide) to reduce cytokine polypeptide (e.g., GM-CSF polypeptide) expression in that T cell. This document also provides methods and materials for using T cells (e.g., CARTs) having a reduced expression level of one or more cytokines (e.g., GM-CSF polypeptides). For example, T cells (e.g., CARTs) having a reduced level of GM-CSF polypeptides can be administered (e.g., in an adoptive cell therapy) to a mammal having cancer to treat the mammal.

In one aspect, this invention provides a method for treating or preventing CAR-T cell related toxicity in a subject in need thereof, the method comprising administering to the subject CAR-T cells having a GM-CSF gene inactivation, GM-CSF gene knock-down or gene knockout (GM-CSF^{k/o} CAR-T cells).

In another aspect, this invention provides a method for increasing CAR-T cell proliferation in a subject treated with GM-CSF-inactivated or GM-CSF^{k/o} CAR-T cells, the method comprising administering to the subject CAR-T cells having a GM-CSF gene inactivation, GM-CSF gene knock-down or gene knockout (GM-CSF^{k/o} CAR-T cells), wherein administration of the GM-CSF^{k/o} CAR-T cells increases CAR-T proliferation in the subject.

In one aspect, this invention provides a method for enhancing anti-tumor efficacy of immunotherapy in a subject, the method comprising administering to the subject CAR-T cells having a GM-CSF gene inactivation, GM-CSF gene knock-down or gene knockout (GM-CSF^{k/o} CAR-T cells), wherein administration of these CAR-T cells improves their anti-tumor efficacy and reduces or prevents immunotherapy-related toxicity.

In another aspect, this invention provides a method for reducing a level of a non-GM-CSF cytokine in a subject treated with immunotherapy, the method comprising administering to the subject CAR-T cells having a GM-CSF gene inactivation, GM-CSF gene knock-down or gene knockout (GM-CSF^{k/o} CAR-T cells).

In one aspect, this invention provides a method for GM-CSF gene inactivation, GM-CSF gene knock-down or GM-CSF knockout (KO) in a cell comprising targeted genome editing or GM-CSF gene silencing.

In one aspect, this invention provides a method for making a chimeric antigen receptor T cell having a reduced level of granulocyte-macrophage colony-stimulating factor (GM-CSF) polypeptides, said method comprising: introducing a nucleic acid construct into an ex vivo T cell, wherein said nucleic acid construct comprises: a) a nucleic acid encoding a guide RNA, wherein said guide RNA is complementary to a GM-CSF messenger RNA; b) a nucleic acid encoding a Cas nuclease, and c) a nucleic acid encoding said chimeric antigen receptor.

In another aspect, this invention provides a method for making a chimeric antigen receptor T cell having a reduced level of granulocyte-macrophage colony-stimulating factor (GM-CSF) polypeptides, said method comprising: introducing a complex into an ex vivo T cell, wherein said complex comprises: a) a guide RNA, wherein said guide RNA is complementary to a GM-CSF messenger RNA; and b) a Cas nuclease; and introducing a nucleic acid encoding said chimeric antigen receptor into said ex vivo T cell.

In an aspect, this invention provides a method for making a chimeric antigen receptor T cell having a reduced level of cytokine polypeptides, said method comprising: introducing a nucleic acid construct into an ex vivo T cell, wherein said nucleic acid construct comprises: a) a nucleic acid encoding a guide RNA, wherein said guide RNA is complementary to a cytokine messenger RNA; b) a nucleic acid encoding a Cas nuclease, and c) a nucleic acid encoding said chimeric antigen receptor.

In another aspect, this invention provides a method for making a chimeric antigen receptor T cell having a reduced level of cytokine polypeptides, said method comprising: introducing a complex into an ex vivo T cell, wherein said complex comprises: a) a guide RNA, wherein said guide RNA is complementary to a cytokine messenger RNA; and b) a Cas nuclease; and introducing a nucleic acid encoding said chimeric antigen receptor into said ex vivo T cell.

In a further aspect, this invention provides a method for improving T cell effector functions of a chimeric antigen receptor T cell, said method comprising: introducing a nucleic acid construct into an ex vivo T cell, wherein said nucleic acid construct comprises: a) a nucleic acid encoding a guide RNA, wherein said guide RNA is complementary to a GM-CSF messenger RNA; b) a nucleic acid encoding a Cas nuclease, and c) a nucleic acid encoding said chimeric antigen receptor.

In an aspect, this invention provides a method for improving T cell effector functions of a chimeric antigen receptor T cell, said method comprising: introducing a complex into an ex vivo T cell, wherein said complex comprises: a) a guide RNA, wherein said guide RNA is complementary to a GM-CSF messenger RNA; and b) a Cas nuclease; and introducing a nucleic acid encoding said chimeric antigen receptor into said ex vivo T cell.

As demonstrated herein, GM-CSF KO CARTs produce reduced levels of GM-CSF and continue to function normally in both in vitro and in vivo models. Also, as demonstrated herein, GM-CSF KO CARTs can have enhanced CART cell function and antitumor activity. For example, enhanced CART cell proliferation and anti-tumor activity can be observed after GM-CSF depletion. CART19 antigen specific proliferation in the presence of monocytes can be increased in vitro after GM-CSF depletion. In ALL patient derived xenografts, CART19 cells can result in a more durable disease control when combined with lenzilumab, and GM-CSF^{k/o} CART cells can be more effective in controlling leukemia in NALM6 xenografts. In some cases, GM-CSF KO CARTs can be incorporated into adoptive T cell therapies (e.g., CART cell therapies) to treat, for example, mammals having cancer without resulting in CRS and/or neurotoxicity. For example, GM-CSF KO CARTs can be incorporated into adoptive T cell therapies (e.g., CART cell therapies) to enhance the therapeutic window after CART cell therapy. In some cases, a single construct can be used both to introduce a CAR into a cell (e.g., a T cell) and to reduce or knock out expression of one or more cytokine polypeptides in that same cell.

In another aspect, this invention provides a method for treating a mammal having cancer, wherein said method comprises administering chimeric antigen receptor T cells having a reduced level of granulocyte-macrophage colony-stimulating factor (GM-CSF) polypeptides to said mammal.

In still another aspect, this invention provides a method for treating a mammal having cancer, wherein said method comprises administering chimeric antigen receptor T cells having a reduced level of cytokine polypeptides to said mammal.

In general, one aspect of this document features methods for making a CART cell having a reduced level of cytokine polypeptides. The methods can include, or consist essentially of introducing a nucleic acid construct into an ex vivo T cell, wherein the nucleic acid construct includes: a) a nucleic acid encoding a guide RNA (gRNA) complementary to a cytokine messenger RNA (mRNA); b) a nucleic acid encoding a Cas nuclease, and c) a nucleic acid encoding a chimeric antigen receptor. The cytokine polypeptides can include granulocyte-macrophage colony-stimulating factor (GM-CSF) polypeptides, interleukin 6 (IL-6) polypeptides, IL-1 polypeptides, M-CSF polypeptides, and/or MIP-1B polypeptides. The cytokine polypeptides can be GM-CSF polypeptides, and the gRNA can include a nucleic acid sequence set forth in SEQ ID NO:1. The Cas nuclease can be a Cas9 nuclease. The nucleic acid encoding the CAR can include a nucleic acid sequence set forth in SEQ ID NO:2. The nucleic acid construct can be a viral vector (e.g., a lentiviral vector). The CAR can target a tumor-associated antigen. (e.g., CD19). The introducing step can include transduction.

In another aspect, this document features methods for making a CAR T cell having a reduced level of cytokine polypeptides. The methods can include, or consist essentially of, introducing a complex into an ex vivo T cell, where the complex includes: a) a gRNA complementary to a cytokine mRNA; and b) a Cas nuclease; and introducing a nucleic acid encoding the CAR into the ex vivo T cell. The cytokine polypeptides can include GM-CSF Polypeptides and/or IL-6 polypeptides. The cytokine polypeptides can be GM-CSF polypeptides, and the gRNA can include a nucleic acid sequence set forth in SEQ ID NO:1. The Cas nuclease can be a Cas9 nuclease. The nucleic acid encoding the CAR can include a nucleic acid sequence set forth in SEQ ID NO:2. The complex can be a ribonucleoprotein (RNP). The CAR can target a tumor-associated antigen (e.g., CD19). The introducing steps can include electroporation.

In another aspect, this document features methods for making a CAR T cell having a reduced level of GM-CSF polypeptides. The methods can include, or consist essentially of introducing a nucleic acid construct into an ex vivo T cell, where the nucleic acid construct includes: a) a nucleic acid encoding a gRNA complementary to a GM-CSF mRNA; b) a nucleic acid encoding a Cas nuclease, and c) a nucleic acid encoding the CAR. The gRNA can include a nucleic acid sequence set forth in SEQ ID NO:1. The Cas nuclease can be a Cas9 nuclease. The nucleic acid encoding the CAR can include a nucleic acid sequence set forth in SEQ ID NO:2. The nucleic acid construct can be a viral vector (e.g., a lentiviral vector). The CAR can target a tumor-associated antigen (e.g., CD19). The introducing step can include transduction.

In another aspect, this document features methods for making a CAR T cell having a reduced level of GM-CSF polypeptides. The methods can include, or consist essentially of, introducing a complex into an ex vivo T cell, where the complex includes: a) a gRNA complementary to a GM-CSF mRNA; and b) a Cas nuclease; and introducing a nucleic acid encoding the CAR into the ex vivo T cell. The gRNA can include a nucleic acid sequence set forth in SEQ ID NO:1. The Cas nuclease can be a Cas9 nuclease. The nucleic acid encoding the CAR can include a nucleic acid sequence set forth in SEQ ID NO:2. The complex can be a RNP. The CAR can target a tumor-associated antigen (e.g., CD19). The introducing steps can include electroporation.

In another aspect, this document features methods for treating a mammal having cancer. The methods can include, or consist essentially of, administering CART cells having a reduced level of cytokine polypeptides to a mammal having cancer. The cytokine polypeptides can include GM-CSF polypeptides and/or IL-6 polypeptides. The cytokine polypeptides can be GM-CSF polypeptides, and the gRNA can include a nucleic acid sequence set forth in SEQ ID NO:1. The mammal can be a human. The cancer can be a lymphoma (e.g., a DLBCL). The cancer can be a leukemia (e.g., an ALL). The CAR can target a tumor-associated antigen (e.g., CD19).

In another aspect, this document features methods for treating a mammal having cancer. The methods can include, or consist essentially of, administering CAR T cells having a reduced level of GM-CSF polypeptides to a mammal having cancer. The mammal can be a human. The cancer can be a lymphoma (e.g., a DLBCL). The cancer can be a leukemia (e.g., ALL). The cancer can be mantle cell lymphoma. The cancer can be follicular lymphoma. The cancer can be multiple myeloma. The CAR can target a tumor-associated antigen (e.g., CD19 or B-cell maturation antigen (BCMA)).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description, examples, and drawings, and from the claims. It should be understood, however, that the detailed description and specific examples while indicating certain embodiments of the invention are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 contains a schematic of an exemplary method of using CRISPR to engineer a GM-CSF knock out (KO) cell. Guide RNA (GACCTGCCTACAGACCCGCC; SEQ ID NO:1) targeting exon 3 of GM-CSF (also known as colony-stimulating factor 2 (CSF2)) was synthesized and cloned into a lentivirus (LV) plasmid. This LV plasmid was used to transduce 293T cells and lentivirus particles were collected at 24 hours and 48 hours and were concentrated. To generate GM-CSF knocked out CART cells, T cells were stimulated with CD3/CD28 beads on day 0. On day 1, T cells were transduced with CAR19 lentivirus particles, and simultaneously with GMCSF knockout CRISPR/Cas9 lentivirus particles. T cells were expanded for 8 days and then harvested.

FIGS. 2A-2B show CAR transduction and GM-CSF knockout efficiency. FIG. 2A contains a graph showing that CRISPR/Cas9 lentivirus with a guide RNA directed to exon 3 of GM-CSF resulted in a knockout efficiency of 24.1%. At the end of the expansion, CART cells were harvested, and DNA was isolated and sent for sequencing to be compared to control sequences. This yielded in a knockout efficiency of 24.1%. FIG. 2B contains a flow cytometric analysis showing that CAR transduction efficiency after transduction with lentivirus was 73%. Flow cytometric analysis was performed on Day 6 after lentivirus transduction.

FIG. 3 shows that GM-CSF KO CART19 cells produce less GM-CSF compared to CART cells, and GM-CSF knockout control T cells produce less amount of GM-CSF compared to control untransduced T cells (UTD). CART19, GM-CSF KO CART19, UTD, or GM-CSF KO UTD were co-cultured with the CD19 positive cell line NALM6 at a ratio of 1:5. 4 hours later, the cells were harvested, permeabilized, and fixed; and intra-cellular staining for cytokines was performed.

FIG. 4 shows that GM-CSF KO CART19 cells expand more robustly compared to CART19. After T cells were transduced with the virus, their expansion kinetics was followed. GM-CSF KO expand more robustly compared to CART19 alone.

FIG. 5 shows an exemplary nucleic acid sequence (SEQ ID NO:2) encoding a CAR targeting CD19 (CAR19).

FIGS. 6A-6D show that GM-CSF neutralization in vitro enhances CAR-T cell proliferation in the presence of monocytes and does not impair CAR-T cell effector function. FIG. 6A contains a graph showing that lenzilumab neutralizes CAR-T cell produced GM-CSF in vitro compared to isotype control treatment as assayed by multiplex after 3 days of culture with CART19 in media alone or CART19 co-cultured with NALM6, n=2 experiments, 2 replicates per experiment, representative experiment depicted, *** p<0.001 between lenzilumab and isotype control treatment, t test, mean±SEM. FIG. 6B contains a graph showing that GM-CSF neutralizing antibody treatment did not inhibit the ability of CAR-T cells to proliferate as assayed by CSFE flow cytometry proliferation assay of live CD3 cells, n=2 experiments, 2 replicates per experiment, representative experiment at 3 day time point depicted, ns p>0.05 between lenzilumab and isotype control treatment, t test, mean±SEM. Alone: CART19 in media alone, MOLM13: CART19+MOLM13, PMA/ION: CART19+5 ng/mL PMA/0.1 μg/mL ION, NALM6: CART19+NALM6. FIG. 6C contains a graph showing that lenzilumab enhanced the proliferation of CART19 compared to isotype control treated with CART19 when co-cultured with monocytes n=3 biologic replicates at 3 day time point, 2 replicates per biological replicate, **** p<0.0001, mean±SEM. FIG. 6D contains a graph showing that lenzilumab treatment did not inhibit cytotoxicity of CART19 or untransduced T cells (UTD) when cultured with NALM6, n=2 experiments, 2 replicates per experiment, representative experiment at 48 hr time point depicted, ns p>0.05 between lenzilumab and isotype control treatment, t test, mean±SEM.

FIGS. 7A-7E show that GM-CSF neutralization in vivo enhances CAR-T cell anti-tumor activity in xenograft models. FIG. 7A contains an experimental schema showing that NSG mice were injected with the CD19+ luciferase+ cell line NALM6 (1×10⁶ cells per mouse I.V). 4-6 days later, mice were imaged, randomized, and received 1-1.5×10⁶ CAR-T 19 or equivalent number of total cells of control UTD cells the following day with either lenzilumab or control IgG (10 mg/Kg, given IP daily for 10 days, starting on the day of CAR-T injection). Mice were followed with serial bioluminescence imaging to assess disease burden beginning day 7 post CAR-T cell injection and were followed for overall survival. Tail vein bleeding was performed 7-8 days after CAR-T cell injection. FIG. 7B contains a graph showing that lenzilumab neutralizes CAR-T produced serum GM-CSF in vivo compared to isotype control treatment as assayed by GM-CSF singleplex, n=2 experiments, 7-8 mice per group, representative experiment, serum from day 8 post CAR-T cell/UTD injection, *** p<0.001 between lenzilumab and isotype control treatment, t test, mean±SEM. FIG. 7C contains a graph showing that lenzilumab treated CAR-T in vivo are equally effective at controlling tumor burden compared to isotype control treated CAR-T in a high tumor burden relapse xenograft model of ALL, day 7 post CAR-T injection, n=2 experiments, 7-8 mice per group, representative experiment depicted, *** p<0.001, * p<0.05, ns p>0.05, t test, mean±SEM. FIG. 7D contains an experimental schema showing that NSG mice were injected with the blasts derived from patients with ALL (1×10⁶ cells per mouse I.V). Mice were bled serially and when the CD19+ cells≥1/uL, mice were randomized to receive 5×10⁶ CART19 (transduction efficiency is around 50%) or UTD cells with either lenzilumab or control IgG (10 mg/Kg, given IP daily for 10 days, starting on the day of CAR-T injection). Mice were followed with serial tail vein bleeding to assess disease burden beginning day 14 post CAR-T cell injection and were followed for overall survival. FIG. 7E contains a graph showing that lenzilumab treatment with CAR-T therapy results in more sustained control of tumor burden over time in a primary acute lymphoblastic leukemia (ALL) xenograft model compared to isotype control treatment with CAR-T therapy, 6 mice per group, ** p<0.01, * p<0.05, ns p>0.05, t test, mean±SEM.

FIG. 8 contains a graph showing that lenzilumab+CAR-T cell treated mice have comparable survival compared to isotype control+CAR-T cell treated mice in a high tumor burden relapse xenograft model of ALL. n=2 experiments, 7-8 mice per group, representative experiment depicted, **** p<0.0001, *** p<0.001, * p<0.05, log-rank.

FIG. 9 contains a graph showing a representative TIDE sequence to verify genome alteration in the GM-CSF CRISPR Cas9 knockout CAR-T cells. n=2 experiments, representative experiment depicted.

FIGS. 10A-10E show that GM-CSF CRISPR knockout CAR-T cells exhibit reduced expression of GM-CSF, similar levels of key cytokines, and enhanced anti-tumor activity. FIG. 10A contains graphs showing that the CRISPR Cas9 GM-CSF^{k/o} CAR-T exhibit reduced GMCSF production compared to wild type CART19, but other cytokine production and degranulation are not inhibited by the GM-CSF gene disruption, n=3 experiments, 2 replicates per experiment, *** p<0.001, * p<0.05, ns p>0.05 comparing GM-CSF k/o CAR-T and CAR-T, t test, mean±SEM. FIG. 10B contains a graph showing that GM-CSF k/o CAR-T have reduced serum human GM-CSF in vivo compared to CAR-T treatment as assayed by multiplex, 5-6 mice per group (4-6 at time of bleed, 8 days post CART injection), **** p<0.0001, *** p<0.001 between GM-CSF k/o CAR-T cells and wild type CAR-T cells, t test, mean±SEM. FIG. 10C contains a graph showing that GM-CSF^{k/o} CART19 in vivo enhances overall survival compared to wild type CART19 in a high tumor burden relapse xenograft model of ALL, 5-6 mice per group, ** p<0.01, log-rank.

FIGS. 10D and 10E contain heat maps showing human (FIG. 10D) and mouse (FIG. 10E) cytokines from multiplex of serum, other than human GM-CSF, show no statistical differences between the GM-CSF k/o CAR-T cells and wild type CAR-T cells, further implicating critical T-cell cytokines aren't adversely depleted by reducing GM-CSF expression, 5-6 mice per group (4-6 at time of bleed), **** p<0.0001, t test.

FIG. 11 contains a graph showing that GM-CSF knockout CAR-T cells in vivo shows slightly enhanced control of tumor burden compared to CAR-Tin a high tumor burden relapse xenograft model of ALL. Days post CAR-T injection listed on x-axis, 5-6 mice per group (2 remained in UTD group at day 13), representative experiment depicted, **** p<0.0001, * p<0.05, 2 way ANOVA, mean±SEM.

FIGS. 12A-12D show that patient derived xenograft model for neurotoxicity and cytokine release syndrome. FIG. 12A contains an experimental schema showing that mice received 1-3×10⁶ primary blasts derived from the peripheral blood of patients with primary ALL. Mice were monitored for engraftment for ˜10-13 weeks via tail vein bleeding. When serum CD19+ cells were 2:10 cells/uL the mice received CART19 (2-5×10⁶ cells) and commenced antibody therapy for a total of 10 days, as indicated. Mice were weighed on a daily basis as a measure of their well-being. Mouse brain MRIs were performed 5-6 days post CART19 injection and tail vein bleeding for cytokine and T cell analysis was performed 4-11 days post CART19 injection, 2 independent experiments. FIG. 12B contains a graph showing that combination of GM-CSF neutralization with CART19 is equally effective as isotype control antibodies combined with CART19 in controlling CD19+ burden of ALL cells, representative experiment, 3 mice per group, 11 days post CART19 injection, * p<0.05 between GM-CSF neutralization+CART19 and isotype control+CART19, t test, mean±SEM. FIG. 12C contains an image showing that brain MRI with CART19 therapy exhibits T1 enhancement, suggestive of brain blood-brain barrier disruption and possible edema. 3 mice per group, 5-6 days post CART19 injection, representative image. FIG. 12D contains graphs showing that high tumor burden primary ALL xenografts treated with CART19 show human CD3 cell infiltration of the brain compared to untreated PDX controls. 3 mice per group, representative image.

FIGS. 13A-13D show that GM-CSF neutralization in vivo ameliorates cytokine release syndrome after CART19 therapy in a xenograft model. FIG. 13A contains a graph showing that lenzilumab and anti-mouse GM-CSF antibody prevent CRS induced weight loss compared to mice treated with CART19 and isotype control antibodies, 3 mice per group, 2 way anova, mean±SEM. FIG. 13B contains a graph showing that human GM-CSF was neutralized in patient derived xenografts treated with lenzilumab and mouse GM-CSF neutralizing antibody, 3 mice per group, *** p<0.001, * p<0.05, t test, mean±SEM. FIG. 13C contains a heat map showing that human cytokines (serum collected 11 days after CART19 injection) exhibit increase in cytokines typical of CRS after CART19 treatment. GM-CSF neutralization results in significant decrease in several cytokines compared to mice treated with CART19 and isotype control antibodies, including several myeloid associated cytokines, as indicated in the panel, 3 mice per group, serum from day 11 post CART19 injection, *** p<0.001, ** p<0.01, * p<0.05, comparing GM-CSF neutralizing antibody treated and isotype control treated mice that received CAR-T cell therapy, t test. FIG. 13D contains a heat map showing that mouse cytokines (serum collected 11 days after CART19 injection) exhibit increase in mouse cytokines typical of CRS after CART19 treatment. GM-CSF neutralization results in significant decrease in several cytokines compared to treated with CART19 with control antibodies, including several myeloid differentiating cytokines, as indicated in the panel, 3 mice per group, serum from day 11 post CART19 injection, *p<0.05, comparing GM-CSF neutralizing antibody treated and isotype control treated mice that received CAR-T cell therapy, t test.

FIGS. 14A-14D show that GM-CSF neutralization in vivo ameliorates neurotoxicity after CART19 therapy in a xenograft model. FIGS. 14A and 14B show that gadolinium enhanced T1-hyperintensity (cubic mm) MRI showed that GM-CSF neutralization helped reduced brain inflammation, blood-brain barrier disruption, and possible edema compared to isotype control (A) representative images, (B) 3 mice per group, ** p<0.01, * p<0.05, 1 way ANOVA, mean±SD. FIG. 14C contains a graph showing that human CD3 T cells were present in the brain after treatment with CART19 therapy. GM-CSF neutralization resulted in a trend toward decreased CD3 infiltration in the brain as assayed by flow cytometry in brain hemispheres, 3 mice per group, mean±SEM. FIG. 14D contains a graph showing that CD1 1b+ bright macrophages were decreased in the brains of mice receiving GM-CSF neutralization during CAR-T therapy compared to isotype control during CAR-T therapy as assayed by flow cytometry in brain hemispheres, 3 mice per group, mean±SEM.

FIGS. 15A-15B show an exemplary generation of GM-CSFk/o CART19 cells. The experimental schema depicts the schema (FIG. 15A), gRNA sequence (FIG. 15B), and primer sequences (FIG. 15B) for generation of GM-CSFk/o CART19. To generate GM-CSFk/o CART19 cells, gRNA was clones into a Cas9 lentivirus vector under the control of a U6 promotor and used for lentivirus production. T cells derived from normal donors were stimulated with CD3/CD28 beads and dual transduced with CAR 19 virus and CRISPR/Cas9 virus 24 hours later. CD3/CD28 magnetic bead removal was performed on Day +6 and GM-CSFk/o CART19 cells or control CART19 cells were cryopreserved on Day 8.

FIG. 16 shows a flow chart for procedures used in RNA sequencing. The binary base call data was converted to fastq using Illumina bcl2fastq software. The adapter sequences were removed using Trimmomatic, and FastQC was used to check for quality. The latest human (GRCh38) and mouse (GRCm38) reference genomes were downloaded from NCBI. Genome index files were generated using STAR30, and the paired end reads were mapped to the genome for each condition. HTSeq was used to generate expression counts for each gene, and DeSeq2 was used to calculate differential expression. Gene ontology was assessed using Enrichr.

FIGS. 17A-17B show respectively, that CD14+ cells are a greater proportion of the CNS cell population in human patients with grade 3 or above neurotoxicity (FIG. 17A) and that anti-hGM-CF antibody, Lenzilumab, caused a reduction in CNS infiltration by CD14+ cells and by CD1 1b+ cells in the primary ALL mouse model used for the NT experiments (FIG. 17B), as detailed in Example 4.

FIGS. 18A-18E show GM-CSF knockout via CRISPR/Cas9 does not impair CART19 production and effector functions. A-B) CRISPR/Cas9 depletion of GM-CSF in CART19 cells generated little to no GM-CSF upon CAR19 stimulation. Representative flow plot (FIG. 18A) or bar graph (FIG. 18B) showing the levels of GM-CSF detected on live CD3+ cells by intracellular flow cytometric staining upon stimulation with CD19+ cell line Nalm6 (one-way ANOVA, **** p<0.0001; 4 biological replicates, 2 technical replicates) FIG. 18C shows CAR19 expression is not impaired by depletion of GM-CSF via CRISPR/Cas9. Representative flow plot showing no differences in CAR19 expression between GM-CSF^{WT} vs GM-CSF^{KO} CART19 cells after CAR19 stimulation via flow cytometric staining. FIG. 18D shows GM-CSF disruption does not affect the composition of CART19 (CD4:CD8 ratio) at rest or upon activation. UTD, GM-CSF^{WT} or GM-CSF^{KO} CART19 cells were co-cultured with either CD19+ cell line Nalm6 (CAR stimulation), CD38/CD28 beads (TCR stimulation) or PMA/Ionomycin (Ca+ influx stimulation) for 5 days, followed by flow cytometric staining of CD4 and CD8 staining (one-way ANOVA, ns=not significant; 2 biological replicates, 2 technical replicates). FIG. 18E shows that CSF KO CART19 show enhanced delayed proliferation. GM-CSF^{KO} and GM-CSF^{WT} CART19 cells were co-cultured with irradiated CD19+ cell line Nalm6, and cell counts were obtained daily for 6 days (one-way ANOVA, * p<0.05; 2 biological replicates).

FIGS. 19A-19G show CRISPR/Cas9 editing of GM-CSF in CART cells is precise and specific. FIG. 19A shows there is not a significant difference in number of single nucleotide variants (SNVs) or insertions and deletions (indels) between GM-CSF KO conditions and controls. Analysis of single nucleotide polymorphisms by whole exome sequencing on three biological replicates (n.s.=not significant; Wilcoxon signed rank test). FIG. 19B shows CSF2 gene-specific editing is precise. Insertion or deletion of cytosine at base pair 132074828 is the only SNV or indel identified in chromosome 5 (CSF2, exon 3) on three biological replicates. FIG. 19C shows potential off targets predicted by available tools are not edited. CCTop predicted targets in exonic regions. Only edit found in our dataset is CSF2 (CRISPRater score: 0.743377). FIGS. 19D-19E show GM-CSF receptors (α and β subunits) are activated upon of T cell and CART19 cells expansion. Untransduced (UTD) T cells were isolated from peripheral blood mononuclear cells (PBMCS) and stimulated over a 6-day expansion period with CD3/CD28 beads. Flow cytometric analysis was performed in order to assess GM-CSF2R α and β subunits expression at days 0, 2, 4 and 6 (Scatter plot (FIG. 19D) and representative flow plot (FIG. 19E), 2 biological replicates). FIGS. 19F-19G show GM-CSF2R α and β subunits are upregulated in activated GM-CSF^{WT} CART19 or GM-CSF^{KO} CART19. GM-CSF_{KO} and GM-CSF^{WT} CART19 cells were activated with either CD3/CD28 beads for 6 days (FIG. 19F) or CD19+ cell line Nalm6 for 24 hours (FIG. 19G). Flow cytometric analysis was performed in order to assess the expression of GM-CSF2R α and β subunits expression on gated on live CD3 (two-way ANOVA; ** p-value<0.01, *** p<0.001, **** p<0.0001).

FIGS. 20A-20F show CRISPR/Cas9-mediated depletion of GM-CSF in CART cells results in decreased T cell apoptosis and AICD. FIG. 20A shows GM-CSF^{WT} CART19 cells are more apoptotic when stimulated through the CAR (CD19+ Nalm6) than TCR (CD3/CD28 beads). CART19 cells were co-cultured with CD19+ cell line Nalm6 (CAR stimulation), CD38/CD28 beads (TCR stimulation) and PMA/Ionomycin (Ca+ influx stimulation). Flow cytometric staining for Annexin V, 7-AAD, and CD3 was performed at 0 hr and 2 hr (two-way ANOVA; ** p-value<0.01, *** p<0.001, **** p<0.0001; 4 biological replicates). FIG. 20B shows representative flow plot of showing the expression of GM-CSF^{WT} CART19 showing apoptotic cells (Annexin V+, 7-AAD−). GM-CSF^{WT} CART19 cells were co-cultured with CD19+ Nalm6. FIGS. 20C-20D show GM-CSF^{KO} CART19 cells are less apoptotic than GM-CSF^{WT} CART19 cells upon stimulation via CAR or non-specifically. GM-CSF^{KO} CART19 and GM-CSF^{WT} CART19 were co-cultured with the CD19+ cell line Nalm6 (FIG. 20C) or PMA/Ionomycin (FIG. 20D). Flow cytometric analysis was performed in order to measure apoptotic cells (Annexin+, 7-AAD−) at 0 hr, 1 hr, 2 hr and 4 hr (two-way ANOVA; ** p-value <0.01, *** p<0.001, **** p<0.0001; 4 biological replicates, 3 technical replicates). FIGS. 20E-20F show GM-CSF disruption ameliorates CART cell apoptosis. GM-CSF^{WT} CART19 and GM-CSF^{KO} CART19 cells were cultured in the presence of irradiated CD19+ cell line Nalm6 for 0 hr, 2 hr, or 6 hr. TUNEL assay via flow cytometry was performed at each time point in order to measure apoptosis based on bromolated deoxyuridine triphosphate nucleotide (BrdU) levels on fragmentated DNA on CART cells (two-way ANOVA; ns=not significant; 2 biological replicates). Bar plot (FIG. 20E) and representative flow plot (FIG. 20F) of % apoptosis by BrdU of G0-G1 phase.

FIGS. 21A-21E show GM-CSF^{KO} CART19 cells exhibit a distinct transcriptomic profile in comparison to GM-CSF^{WT} CART19 cells. A-C) Comparison of gene expression between untransduced T cells (UTD), CART19 cells and GM-CSF^{KO} CART19 cells via RNA-seq. Differential expression by heatmap (FIG. 21A), volcano plot (FIG. 21B), or principal component analysis (FIG. 21C) on RNA isolated from untransduced T cells (UTD), CART19, and GM-CSF^{KO} CART19 cells on day 8 of CART expansion (adj. p-value<0.05, 3 biological replicates). FIG. 21D shows apoptotic pathways are enriched in GM-CSF^{KO} CART19. Gene set enrichment analysis of significantly downregulated genes using Enrichr (p-value<0.05). FIG. 21E shows apoptosis is not impaired on CART19 cells that were produced in the presence of anti-GM-CSF antibody. CART19 or CART19 cells generated in the presence of anti-GM-CSF antibody were co-cultured in the presence of CD19+ cell line Nalm6 and flow cytometric staining for Annexin V, 7-AAD, and CD3 was performed at 0 hr, 1 hr, 2 h,r 4 hr, and 4 hr (two-way ANOVA; ns=not significant; 3 biological replicates, 3 technical replicates).

FIGS. 22A-22N show GM-CSF disruption on CART19 modulates its early activation and anti-tumor activity. FIGS. 22A-22H show GM-CSF^{KO} CART19 cells showed altered expression levels of T cell activation markers. GM-CSF^{KO} or GM-CSF^{WT} CART19 cells were co-cultured with CD19+ Nalm6 for 24 hrs and flow cytometric staining is performed in order to measure CD3 (FIG. 22A), CD45 (FIG. 22B), CD69 (FIGS. 22C and 22D), HLA-DR (FIGS. 22E and 22F) and CD25 (FIGS. 22G and 22H) (one-way ANOVA; ** p<0.01, *** p<0.001, **** p <0.0001; 2 biological replicates, 2 technical replicates). FIGS. 22I-22L show GM-CSF disruption reduces early CART cell activation and shows prolonged expansion in an in vivo JeKo-1 relapse xenograft model. Experimental schema showing NSG mice engrafted with the CD19+ luciferase+cell line JeKo-1 (1×10⁶ cells intravenous [i.v.] and randomized to treatment with UTD T cells, GM-CSF^{WT} CART19 cells, and GM-CSF^{KO} CART19 cells (1×10⁶ cells i.v.) (FIG. 22I). Tumor burden by bioluminescent imaging over 20 days after CART cell therapy (two-way ANOVA; *** p<0.001) (FIG. 22J). FIG. 22K shows GM-CSF^{KO} CART19 cells reduced activation in vivo. Peripheral blood analysis of UTD, GM-CSF^{WT} CART19 and GM-CSF^{KO} CART19 cells where CD69, HLA-DR, and CD25 were measured by flow cytometry (one-way ANOVA, * p<0.05). FIG. 22L shows GM-CSF^{KO} CART19 cells exhibit enhanced delayed proliferation in vivo. Peripheral blood analysis of UTD, GM-CSF^{WT} CART19 and GM-CSF^{KO} CART19 cells where CD3+ cells were quantified (one-way ANOVA, *** p<0.001). FIG. 22M shows GM-CSF^{KO} CART19 cells exhibit prolonged survival in vivo. FIG. 22N shows GM-CSF^{KO} CART19 cells exhibit reduced expression of TRAIL-R1. GM-CSF^{WT} and GM-CSF^{KO} CART19 are co-cultured with CD19+ Nalm6 for 24 hours and flow cytometric staining is performed (one-way ANOVA, ****<0.0001; 2 biological replicates).

FIGS. 23A-23D show reduced CART19 cell apoptosis following GM-CSF disruption is due to modulation of intrinsic, and not extrinsic, apoptosis pathways. FIGS. 23A-23B show there is no difference in apoptotic levels between GM-CSF^{WT} CART19 or GM-CSF^{KO} CART19 when death receptors Fas or TRAIL-R2 (DR5) are blocked. GM-CSF^{WT} CART19 or GM-CSF^{KO} CART19 cells were co-cultured with CD19+ cell line Nalm6 in the presence of either an IgG Isotype control or a monoclonal antibody against Fas or TRAIL-R2 (10 ng/mL). Flow cytometric staining for Annexin V, 7-AAD, and CD3 was performed after 24 hours (two-way ANOVA; ** p-value <0.01, *** p<0.001, **** p<0.0001; 3 biological replicates, 2 technical replicates). FIGS. 23C-23D show GM-CSF disruption ameliorates CART cell apoptosis through modulation of intrinsic pathways. GM-CSF^{WT} CART19 and GM-CSF^{KO} CART19 cells were co-cultured with CD19+ Nalm6 and western blot for BID was performed at 0 hr, 2 hr, 4 hr and 6 hr (two-way ANOVA; ** p-value <0.01, *** p<0.001, **** p<0.0001; 2 biological replicates).

FIGS. 24A-24C show a schematic of GM-CSF^{KO} CART19 production with a CRISPR/Cas9 lentiviral vector, similarity of GM-CSF^{KO} CART19 cells and GM-CSF^{WT} CART19 cells in killing assay and a schematic of CART19 production in the presence of GM-CSF blocking antibody. FIG. 24A shows schema of GM-CSF^{KO} CART19 production with CRISPR/Cas9 lentiviral vector. FIG. 24B shows GM-CSF^{WT} CART19 or GM-CSF^{KO} CART19 cells do not show a difference in killing (two-way ANOVA; ns=not significant). FIG. 24C shows schema of CART19 production in the presence of GM-CSF blocking antibody.

### DETAILED DESCRIPTION OF THE INVENTION

The present invention may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

Despite the remarkable activity of CD19-directed chimeric antigen receptor T cell (CART19) therapy in treating B cell malignancies, limitations include 1) the development of life-threatening complications such as neurotoxicity (NT) and cytokine release syndrome (CRS) and 2) lack of durable response. Emerging literature suggests that inhibitory myeloid cells and their cytokines play an important role in inducing CART cell toxicities and contribute to CART inhibition. Specifically, and of relevance to the disclosure herein, granulocyte-macrophage colony-stimulating factor (GM-CSF) was found to be implicated in the development of NT and CRS after CART19 therapy based on correlative studies from pivotal clinical trials.

GM-CSF is produced by macrophages, T cells, NK cells, endothelial cells and fibroblasts and plays several roles in the hematopoietic and immune system. GM-CSF plays a redundant role in stimulating stem cells to differentiate into monocytes, granulocytes, and neutrophils. GM-CSF also activates monocytes and differentiates them into macrophages and is a component of the immune response to infections. Following allogeneic transplantation, GM-CSF has also been demonstrated to drive graft versus host pathology by licensing donor derived myeloid cells to produce inflammatory mediators such as interleukin 1(3. GM-CSF was also shown to recruit dendritic cells and promote graft versus host disease, amplifying the activation of alloreactive T cells.

In a recent analysis of the pivotal clinical trial ZUMA-1, which led to FDA approval of axicabtagene ciloleucel (Axi-cel; Yescarta ®) CART cell therapy, GM-CSF was the most significant cytokine associated with the development of CRS and NT. GM-CSF was found to be elevated early (within the first 24-48 hours) following CART19 infusion, suggesting a potential role in initiation and/or propagation of CART cell associated toxicities. Preclinical studies identified that depletion of GM-CSF prevents CRS and NT and enhances CART cell anti-tumor activity in preclinical models. Specifically, these models indicate that GM-CSF neutralization with lenzilumab reduces monocyte activation and decreases inhibitory myeloid cytokines, which in turn ameliorates the development of CRS, preserves blood brain barrier integrity, and prevents neuroinflammation. This preclinical work led to the launch of ZUMA-19, an ongoing phase Ib/II multi-center study of Axi-cel CART19 in a sequenced therapy with lenzilumab for GM-CSF neutralization in patients with relapsed or refractory large B-cell lymphoma (NCT04314843).

While GM-CSF is secreted primarily by myeloid cells and contributes to their activation, it is also produced by T cells. Therefore, it was hypothesized that disruption of GM-CSF during CART cell manufacturing would result in reduced GM-CSF levels and decreased monocyte activation. Indeed, it was shown that CRISPR/Cas9 gene disruption of GM-CSF in CART cells during their manufacturing generates GM-CSF^{k/o} CART19 cells, which produce less GM-CSF while maintaining their effector functions. This work was recently corroborated by a preclinical study using TALENs to knock out GM-CSF. In fact, the results indicate that GM-CSF^{k/o} CART19 cells exhibit superior antitumor activity in xenograft models where myeloid cells are lacking. This pointed to a direct impact of GM-CSF on T cells, independent of its role as a mediator of monocyte activation and monocyte induced T cell inhibition. To this end, GM-CSF producing T cells (Th GM) have been identified as a novel subset of T cells with unique phenotypic and functional properties. Specifically, Th^{GM} cells were found to induce activation of other T cell subsets, thus amplifying T cell responses. Th^{GM} were also demonstrated to be more susceptible to apoptosis and activation-induced cell death (AICD).

It was, therefore, hypothesized that GM-CSF depletion in CART cells results in reduced AICD and enhanced anti-tumor activity independent of the effect on myeloid cell activation. In Example 6, how GM-CSF disruption of CART cells impact their functions directly was tested.

This document provides methods and materials for generating T cells (e.g., chimeric antigen receptor (CAR) T cells (CARTs)) having a reduced expression level of one or more cytokine polypeptides (e.g., GM-CSF polypeptides). In some cases, a T cell (e.g., CART) can be engineered to knock out (KO) a nucleic acid encoding a GM-CSF polypeptide to reduce GM-CSF polypeptide expression in that T cell (e.g., as compared to a T cell that is not engineered to KO a nucleic acid encoding a GM-CSF polypeptide). A T cell that is engineered to KO a nucleic acid encoding a GM-CSF polypeptide can also be referred to herein as a GM-CSF KO T cell. In some cases, the methods and materials provided herein can be used to modulate myeloid cells. In some cases, the methods and materials provided herein can be used to deplete myeloid cells. In some cases, the methods and materials provided herein can be used to enhance T cell (e.g., CARTs) efficacy.

T cells (e.g., CARTs) provided herein can be designed to have a reduced expression level of any appropriate cytokine polypeptide or combination of cytokine polypeptides. For example, a T cell (e.g., a CART) provided herein can be designed to have a reduced expression level of a GM-CSF polypeptide, an interleukin 6 (IL-6) polypeptide, a G-CSF, a interferon gamma (IFN-g) polypeptide, an IL-1B polypeptide, an IL-10 polypeptide, a monocyte chemoattractant protein 1 (MCP-1) polypeptide, a monokine induced by gamma (MIG) polypeptide, a macrophage inflammatory protein (MIP) polypeptide (e.g., a MIP-1β polypeptide), a tumor necrosis factor alpha (TNF-a) polypeptide, an IL-2 polypeptide, a perforin polypeptide, or any combination thereof. For example, a T cell can be designed to have a reduced expression level of both GM-CSF and IL-6 polypeptides.

In one aspect, this invention provides a method for enhancing anti-tumor efficacy of immunotherapy in a subject, the method comprising administering to the subject CAR-T cells having a GM-CSF gene inactivation, GM-CSF gene knock-down or gene knockout (GM-CSF^{k/o} CAR-T cells), wherein administration of the CAR-T cells improves anti-tumor efficacy and reduces or prevents immunotherapy-related toxicity. In an embodiment of the herein provided method, the method further comprises administering to the subject an anti-hGM-CSF antibody, wherein the anti-hGM-CSF antibody is a recombinant anti-hGM-CSF antibody that binds to and neutralizes human GM-CSF. In a particular embodiment, the method comprises administering to the subject an anti-hGM-CSF antibody. In another embodiment, the immunotherapy-related toxicity CAR-T comprises Cytokine Release Syndrome (CRS), neurotoxicity (NT), neuroinflammation or a combination thereof.

In certain embodiments of the herein provided methods, the administration of (i) the CAR-T cells having a GM-CSF gene inactivation, GM-CSF gene knock-down or gene knockout (GM-CSF^{k/o} CAR-T cells) or (ii) the CAR-T cells and the anti-hGM-CSF antibody decreases or prevents CD14+ myeloid cell trafficking to a central nervous system (CNS) of the subject. In an embodiment, a high level of CD14+ myeloid cells in the central nervous system (CNS) of the subject is indicative of neurotoxicity. A level of CD14+ myeloid cells in the CNS is determined by performing a lumbar puncture, removing a sample of cerebrospinal fluid (CSF), and measuring the CD14+ cells in the CSF, for example by cytometric flow analysis (Flow Cytometry), ELISA, anti-CD14-FITC monoclonal antibody or other suitable measurement techniques.

In an embodiment of the herein provided methods, an objective response rate of the subject administered the anti-hGM-CSF antibody is improved compared to a subject that is not administered the anti-hGM-CSF antibody. In a specific embodiment, the objective response rate is a complete response rate or a partial response rate. In another embodiment, a progression free response and/or survival of the subject is improved compared to a subject that is not administered the anti-hGM-CSF antibody and/or the CAR-T cells having a GM-CSF gene inactivation, GM-CSF gene knock-down or gene knockout (GM-CSF^{k/o} CAR-T cells). In a further embodiment, the survival is overall survival of the subject. In another embodiment, the anti-hGM-CSF antibody is administered to the subject before, during or after administration of the CAR-T cells having a GM-CSF gene inactivation, GM-CSF gene knock-down or GM-CSF^{k/o} CAR-T cells.

In a particular embodiment of the herein provided methods, the immunotherapy comprises administering chimeric antigen receptor-expressing T-cells (CAR T-cells). In an embodiment, wherein the CAR T-cells are CART19 cells. In another embodiment of the herein provided methods, of claim 1, the immunotherapy comprises adoptive cell transfer selected from the group consisting of administering T-cell receptor (TCR) modified T-cells, tumor-infiltrating lymphocytes (TIL), chimeric antigen receptor (CAR)-modified natural killer cells, or dendritic cells, or any combination thereof. In some embodiments, the immunotherapy comprises administration of a monoclonal antibody, a cytokine, a cancer vaccine, a T cell engaging bispecific antibody, or any combination thereof. In a particular embodiment, the subject has a cancer. In certain embodiments, the cancer is lymphoma or a leukemia. In another embodiment, the lymphoma is a diffuse large B cell lymphoma (DLBCL). In still another embodiment, the leukemia is acute lymphoblastic leukemia (ALL). In still another embodiment, the lymphoma is mantle cell lymphoma. In still another embodiment, the lymphoma is follicular lymphoma. In still another embodiment, the cancer is multiple myeloma.

In another aspect, this invention provides a method for reducing a level of a non-GM-CSF cytokine in a subject treated with immunotherapy, the method comprising administering to the subject CAR-T cells having a GM-CSF gene inactivation, GM-CSF gene knock-down or gene knockout (GM-CSF^{k/o} CAR-T cells). In a specific embodiment of the herein provided method, the method further comprising administering to the subject an anti-hGM-CSF antibody to the subject. In another embodiment, the non-GM-CSF cytokine is IP-10, IL-la, IL-lb, IL-2, IL-3, IL-4, IL-5, IL-6, IL-1Ra, IL-9, IL-10, VEGF, TNF-a, FGF-2, IFN-γ, IL-12p40, IL-12p70, sCD40L, KC, MDC, MCP-1, MIP-la, MIP-lb or a combination thereof. In another embodiment of the herein provided methods, the immunotherapy-related toxicity CAR-T comprises CRS, NT, neuroinflammation or a combination thereof.

In one aspect, this invention provides a method for treating or preventing CAR-T cell related toxicity in a subject in need thereof, the method comprising administering to the subject CAR-T cells having a GM-CSF gene inactivation, GM-CSF gene knock-down or gene knockout (GM-CSF^{k/o} CAR-T cells). In some embodiments of the herein provided methods, the CAR-T cell related toxicity comprises neurotoxicity, cytokine release syndrome (CRS) or a combination thereof. In particular embodiments, the subject has a cancer and/or a tumor. In an embodiment, the cancer is lymphoma or a leukemia. In certain embodiments, the lymphoma is a diffuse large B cell lymphoma (DLBCL). In some embodiments, the leukemia is acute lymphoblastic leukemia (ALL). In still another embodiment, the lymphoma is mantle cell lymphoma. In still another embodiment, the lymphoma is follicular lymphoma. In still another embodiment, the cancer is multiple myeloma.

In an embodiment of the herein provided methods, levels of the CAR-T cells having a GM-CSF gene inactivation, GM-CSF gene knock-down or gene knockout (GM-CSF^{k/o} CAR-T cells) expand and persist in blood of the subject from a peak level of GM-CSF^{k/o} CAR-T cell expansion during the first 30 days after administration of the GM-CSF^{k/o} CAR-T cells and expansion of the GM-CSF^{k/o} CAR-T cells up to at least 90 days to 180 days after the administration of the GM-CSF^{k/o} CAR-T cells. In some embodiments, GM-CSF^{k/o} CAR-T cell expansion and persistence in the blood of the subject continues for up to 24 months after administration of the GM-CSF^{k/o} CAR-T cells. In certain embodiments, GM-CSF^{k/o} CAR-T cell expansion and persistence in the blood of the subject achieves an anti-cancer or anti-tumor efficacy from 90 days to 24 months after administration of the GM-CSF^{k/o} CAR-T cells. In a particular embodiment, the anti-cancer or anti-tumor efficacy in the subject is a complete or partial remission of the cancer and/or the tumor. In further particular embodiments, the anti-cancer or anti-tumor efficacy in the subject is a reduction or an absence of signs and symptoms of the cancer and/or the tumor.

In another aspect, this invention provides a method for increasing CAR-T cell proliferation in a subject treated with GM-CSF-inactivated or GM-CSF^{k/o} CAR-T cells, the method comprising administering to the subject CAR-T cells having a GM-CSF gene inactivation, GM-CSF gene knock-down or gene knockout (GM-CSF^{k/o} CAR-T cells), wherein administration of the GM-CSF^{k/o}CAR-T cells increases CAR-T proliferation in the subject. In some embodiments, of the herein provided methods, the administration of the GM-CSF^{k/o}CAR-T cells and expansion of the GM-CSF^{k/o} CAR-T cells reduces the overall production of GM-CSF by CAR T cells by 75%-95%. In an embodiment of the herein provided methods, the administration of the GM-CSF^{k/o} CAR-T cells and expansion of the GM-CSF^{k/o} CAR-T cells reduces the overall production of GM-CSF by CAR T cells by 95%-99% or eliminates production of GM-CSF by the GM-CSF^{k/o} CAR-T cells. In some embodiments, production of GM-CSF by the administered GM-CSF^{k/o} CAR T cells is completely eliminated. In some embodiments of the herein provided methods, reduction or elimination of the production of GM-CSF by the GM-CSF^{k/o} CAR-T cells increases production and expansion of the GM-CSF by the GM-CSF^{k/o} CAR-T cells. In certain \embodiments, increased production and expansion of the GM-CSF by the GM-CSF^{k/o} CAR-T cells reduces of eliminates CAR-T cell related toxicity in the subject, wherein the CAR-T cell related toxicity comprises neurotoxicity, cytokine release syndrome (CRS) or a combination thereof. In a particular embodiment, the subject has a cancer and/or a tumor. In some embodiments, the cancer is lymphoma or a leukemia. In certain \embodiments, the lymphoma is a diffuse large B cell lymphoma (DLBCL). In some embodiments, the leukemia is acute lymphoblastic leukemia (ALL). In still another embodiment, the lymphoma is mantle cell lymphoma. In still another embodiment, the lymphoma is follicular lymphoma. In still another embodiment, the cancer is multiple myeloma. In an embodiment of the herein provided methods, levels of the CAR-T cells having a GM-CSF gene inactivation, GM-CSF gene knock-down or gene knockout (GM-CSF^{k/o} CAR-T cells) expand and persist in blood of the subject from a peak level of GM-CSF^{k/o} CAR-T cell expansion during the first 30 days after administration of the GM-CSF^{k/o} CAR-T cells and expansion of the GM-CSF^{k/o} CAR-T cells up to at least 90 days to 180 days after the administration of the GM-CSF^{k/o} CAR-T cells. In particular embodiments, GM-CSF^{k/o} CAR-T cell expansion and persistence in the blood of the subject continues for up to 24 months after administration of the GM-CSF^{k/o} CAR-T cells. In certain embodiments, GM-CSF^{k/o} CAR-T cell expansion and persistence in the blood of the subject achieves an anti-cancer or anti-tumor efficacy from 90 days to 24 months after administration of the GM-CSF^{k/o} CAR-T cells. In some embodiments, the anti-cancer or anti-tumor efficacy in the subject is a complete or partial remission of the cancer and/or the tumor. In a particular embodiment, the anti-cancer or anti-tumor efficacy in the subject is a reduction or an absence of signs and symptoms of the cancer and/or the tumor.

In one aspect, this invention provides a method for GM-CSF gene inactivation, GM-CSF gene knock-down or GM-CSF knockout (KO) in a cell comprising targeted genome editing or GM-CSF gene silencing. In an embodiment of the herein provided method, the method further comprises an endonuclease as a nucleic acid cutting enzyme. In some embodiments, the endonuclease is a Fok 1 restriction enzyme or a flap endonuclease 1 (FEN-1). In certain embodiments, the endonuclease is a Cas9 CRISPR associated protein 9 (Cas9). In a specific embodiment, the GM-CSF gene inactivation by CRISPR/Cas9 targets and edits a GM-CSF gene at Exon 1, Exon 2, Exon 3 or Exon 4. In an embodiment, the GM-CSF gene inactivation comprising CRISPR/Cas9 targets and edits the GM-CSF gene at Exon 3. In another embodiment, the GM-CSF gene inactivation comprising CRISPR/Cas9 targets and edits the GM-CSF gene at Exon 1.

In still another embodiment, the GM-CSF gene inactivation comprises multiple CRISPR/Cas9 enzymes, wherein each Cas9 enzyme targets and edits a different sequence of the GM-CSF gene at Exon 1, Exon 2, Exon 3 or Exon 4. In another embodiment, the GM-CSF gene inactivation comprises bi-allelic CRISPR/Cas9 targeting and knockout/inactivation of the GM-CSF genes.

In a particular embodiment of the herein provided methods, the method further comprises treating primary T cells with valproic acid to enhance bi-allele gene knockout/inactivation. In another embodiment the targeted genome editing comprises Zinc finger (ZnF) proteins. In certain embodiments, the targeted genome editing comprises transcription activator-like effector nucleases (TALENS). In particular embodiments, the targeted genome editing comprises a homing endonuclease, wherein the homing endonuclease is an ARC nuclease (ARCUS) or a meganuclease. In an embodiment of the herein provided methods, the targeted genome editing comprises a flap endonuclease (FEN-1). In a specific embodiment, the cell is a CAR T cell. In a particular embodiment, the CAR T cell is a CD19 CAR-T cell. In another embodiment, the CAR T cell is a BCMA CAR-T cell. In another embodiment, the GM-CSF gene silencing is selected from the group consisting of RNA interference (RNAi), short interfering RNS (siRNA), and DNA-directed RNA interference (ddRNAi).

In one aspect, this invention provides a method for making a chimeric antigen receptor T cell having a reduced level of granulocyte-macrophage colony-stimulating factor (GM-CSF) polypeptides, said method comprising: introducing a nucleic acid construct into an ex vivo T cell, wherein said nucleic acid construct comprises: a) a nucleic acid encoding a guide RNA, wherein said guide RNA is complementary to a GM-CSF messenger RNA; b) a nucleic acid encoding a Cas nuclease, and c) a nucleic acid encoding said chimeric antigen receptor. In an embodiment of said herein provided method, said guide RNA comprises a nucleic acid sequence set forth in SEQ ID NO: 1. In another embodiment, said Cas nuclease is Cas9 nuclease. In a further embodiment, said nucleic acid encoding said chimeric antigen receptor comprises a nucleic acid sequence set forth in SEQ ID NO:2. In another embodiment, said nucleic acid construct is a viral vector. In a particular embodiment, said viral vector is a lentiviral vector. In a further embodiment, said chimeric antigen receptor targets a tumor-associated antigen. In another embodiment, said tumor-associated antigen is CD19. In a further embodiment, said introducing step comprises transduction.

In another aspect, this invention provides a method for making a chimeric antigen receptor T cell having a reduced level of granulocyte-macrophage colony-stimulating factor (GM-CSF) polypeptides, said method comprising: introducing a complex into an ex vivo T cell, wherein said complex comprises: a) a guide RNA, wherein said guide RNA is complementary to a GM-CSF messenger RNA; and b) a Cas nuclease; and introducing a nucleic acid encoding said chimeric antigen receptor into said ex vivo T cell. In an embodiment of the herein provided method, said guide RNA comprises a nucleic acid sequence set forth in SEQ ID NO:1. In another embodiment, said Cas nuclease is Cas9 nuclease. In a particular embodiment, said nucleic acid encoding said chimeric antigen receptor comprises a nucleic acid sequence set forth in SEQ ID NO:2. In another embodiment, said complex is a ribonucleoprotein. In a further embodiment, said chimeric antigen receptor targets a tumor-associated antigen. In a particular embodiment, said tumor-associated antigen is CD19. In another embodiment of the herein provided method, said introducing steps comprise electroporation.

In another aspect, this invention provides a method for treating a mammal having cancer, wherein said method comprises administering chimeric antigen receptor T cells having a reduced level of granulocyte-macrophage colony-stimulating factor (GM-CSF) polypeptides to said mammal. In a particular embodiment, said mammal is a human. In another embodiment, said cancer is a lymphoma. In a further embodiment, said lymphoma is a diffuse large B cell lymphoma. In another embodiment, said cancer is a leukemia. In another embodiment, said leukemia is an acute lymphoblastic leukemia. In still another embodiment, the lymphoma is mantle cell lymphoma. In still another embodiment, the lymphoma is follicular lymphoma. In still another embodiment, the cancer is multiple myeloma. In an embodiment, said chimeric antigen receptor targets a tumor-associated antigen. In a particular embodiment, said tumor-associated antigen is CD19. In a particular embodiment, the tumor-associated antigen is BMCA.

In an aspect, this invention provides a method for making a chimeric antigen receptor T cell having a reduced level of cytokine polypeptides, said method comprising: introducing a nucleic acid construct into an ex vivo T cell, wherein said nucleic acid construct comprises: a) a nucleic acid encoding a guide RNA, wherein said guide RNA is complementary to a cytokine messenger RNA; b) a nucleic acid encoding a Cas nuclease, and c) a nucleic acid encoding said chimeric antigen receptor. In certain embodiments of said herein provided method, said cytokine polypeptides comprise granulocyte-macrophage colony-stimulating factor (GM-CSF) polypeptides and/or interleukin 6 (IL-6) polypeptides. In a particular embodiment, said cytokine polypeptides are GM-CSF polypeptides, and wherein said guide RNA comprises a nucleic acid sequence set forth in SEQ ID NO:1. In an embodiment, said Cas nuclease is Cas9 nuclease. In another embodiment, said nucleic acid encoding said chimeric antigen receptor comprises a nucleic acid sequence set forth in SEQ ID NO:2. In a further embodiment, said nucleic acid construct is a viral vector. In a specific embodiment, said viral vector is a lentiviral vector. In another embodiment, said chimeric antigen receptor targets a tumor-associated antigen. In various embodiments, said tumor-associated antigen is CD19. In another embodiment, said introducing step comprises transduction.

In another aspect, this invention provides a method for making a chimeric antigen receptor T cell having a reduced level of cytokine polypeptides, said method comprising: introducing a complex into an ex vivo T cell, wherein said complex comprises: a) a guide RNA, wherein said guide RNA is complementary to a cytokine messenger RNA; and b) a Cas nuclease; and introducing a nucleic acid encoding said chimeric antigen receptor into said ex vivo T cell. In an embodiment of said herein provided method, said cytokine polypeptides comprise granulocyte-macrophage colony-stimulating factor (GM-CSF) polypeptides and/or interleukin 6 (IL-6) polypeptides. In a particular embodiment, said cytokine polypeptides are GM-CSF polypeptides, and wherein said guide RNA comprises a nucleic acid sequence set forth in SEQ ID NO:1. In another embodiment, said Cas nuclease is Cas9 nuclease. In another embodiment, said nucleic acid encoding said chimeric antigen receptor comprises a nucleic acid sequence set forth in SEQ ID NO:2. In a further embodiment, said complex is a ribonucleoprotein. In a still further embodiment, said chimeric antigen receptor targets a tumor-associated antigen. In a specific embodiment, said tumor-associated antigen is CD19. In another embodiment, said introducing steps comprises electroporation.

In still another aspect, this invention provides a method for treating a mammal having cancer, wherein said method comprises administering chimeric antigen receptor T cells having a reduced level of cytokine polypeptides to said mammal. In an embodiment of said provided method, said cytokine polypeptides comprise granulocyte-macrophage colony-stimulating factor (GM-CSF) polypeptides and/or interleukin 6 (IL-6) polypeptides. In another embodiment, said cytokine polypeptides are GM-CSF polypeptides, and wherein said guide RNA comprises a nucleic acid sequence set forth in SEQ ID NO:1. In a further embodiment, said mammal is a human. In another embodiment, said cancer is a lymphoma. In still another embodiment, said lymphoma is a diffuse large B cell lymphoma. In another embodiment, said cancer is a leukemia. In a further embodiment, said leukemia is an acute lymphoblastic leukemia. In still another embodiment, the lymphoma is mantle cell lymphoma. In still another embodiment, the lymphoma is follicular lymphoma. In still another embodiment, the cancer is multiple myeloma. In an embodiment, said chimeric antigen receptor targets a tumor-associated antigen. In a particular embodiment, said tumor-associated antigen is CD19. In a particular embodiment, said tumor-associated antigen is BCMA.

In one aspect, this invention provides a method for improving T cell effector functions of a chimeric antigen receptor T cell, said method comprising: introducing a nucleic acid construct into an ex vivo T cell, wherein said nucleic acid construct comprises: a) a nucleic acid encoding a guide RNA, wherein said guide RNA is complementary to a GM-CSF messenger RNA; b) a nucleic acid encoding a Cas nuclease, and c) a nucleic acid encoding said chimeric antigen receptor.

In another aspect, this invention provides a method for improving T cell effector functions of a chimeric antigen receptor T cell, said method comprising: introducing a complex into an ex vivo T cell, wherein said complex comprises: a) a guide RNA, wherein said guide RNA is complementary to a GM-CSF messenger RNA; and b) a Cas nuclease; and introducing a nucleic acid encoding said chimeric antigen receptor into said ex vivo T cell.

The term “reduced level” as used herein with respect to an expression level of a cytokine (e.g., GM-CSF) refers to any level that is lower than a reference expression level of that cytokine (e.g., GM-CSF). The term “reference level” as used herein with respect to a cytokine (e.g., GM-CSF) refers to the level of that cytokine (e.g., GM-CSF) typically observed in a sample (e.g., a control sample) from one or more mammals (e.g., humans) not engineered to have a reduced expression level of that cytokine (e.g., GM-CSF polypeptides) as described herein. Control samples can include, without limitation, T cells that are wild-type T cells (e.g., T cells that are not GM-SCF KO T cells). In some cases, a reduced expression level of a cytokine polypeptide (e.g., a GM-CSF polypeptide) can be an undetectable level of that cytokine (e.g., GM-CSF). In some cases, a reduced expression level of GM-CSF polypeptides can be an eliminated level of GM-CSF.

In some cases, a T cell having (e.g., engineered to have) a reduced expression level of one or more cytokine polypeptides such as a GM-CSF KO T cell can maintain normal T cell functions such as T cell degranulation and release of cytokines (e.g., as compared to a CART that is not engineered to have a reduced expression level of that cytokine (e.g., GM-CSF polypeptides) as described herein).

In some cases, a T cell having (e.g., engineered to have) a reduced level of GM-CSF polypeptides (e.g., a GM-CSF KO T cell) can have enhanced CART function such as antitumor activity, proliferation, cell killing, cytokine production, exhaustion susceptibility, antigen specific effector functions, persistence, and differentiation (e.g., as compared to a CART that is not engineered to have a reduced level of GM-CSF polypeptides as described herein).

In some cases, a T cell having (e.g., engineered to have) a reduced level of GM-CSF polypeptides (e.g., a GM-CSF KO T cell) can have enhanced T cell expansion (e.g., as compared to a CART that is not engineered to have a reduced level of GM-CSF polypeptides as described herein).

A T cell having (e.g., engineered to have) a reduced expression level of one or more cytokines (e.g., a GM-CSF polypeptide) such as a GM-CSF KO T cell can be any appropriate T cell. A T cell can be a naive T cell. Examples of T cells that can be designed to have a reduced expression level of one or more cytokines as described herein include, without limitation, cytotoxic T cells (e.g., CD4+ CTLs and/or CD8+ CTLs). For example, a T cell that can be engineered to have a reduced level of GM-CSF polypeptides as described herein can be a CART. In some cases, one or more T cells can be obtained from a mammal (e.g., a mammal having cancer). For example, T cells can be obtained from a mammal to be treated with the materials and method described herein.

A T cell having (e.g., engineered to have) a reduced expression level of one or more cytokine polypeptides (e.g., a GM-CSF polypeptide) such as a GM-CSF KO T cell can be generated using any appropriate method. In some cases, a T cell (e.g., CART) can be engineered to KO a nucleic acid encoding a GM-CSF polypeptide to reduce GM-CSF polypeptide expression in that T cell.

In some cases, when a T cell (e.g., CART) is engineered to KO a nucleic acid encoding a cytokine (e.g., a GM-CSF polypeptide) to reduce expression of that cytokine polypeptide in that T cell, any appropriate method can be used to KO a nucleic acid encoding that cytokine. Examples of techniques that can be used to knock out a nucleic acid sequence encoding a cytokine polypeptide (e.g., a GM-CSF polypeptide) include, without limitation, gene editing, homologous recombination, non-homologous end joining, and microhomology end joining. For example, gene editing (e.g., with engineered nucleases) can be used to KO a nucleic acid encoding a GM-CSF polypeptide. Nucleases useful for genome editing include, without limitation, CRISPR-associated (Cas) nucleases, zinc finger nucleases (ZFNs), transcription activator-like effector (TALE) nucleases, and homing endonucleases (HE; also referred to as meganucleases).

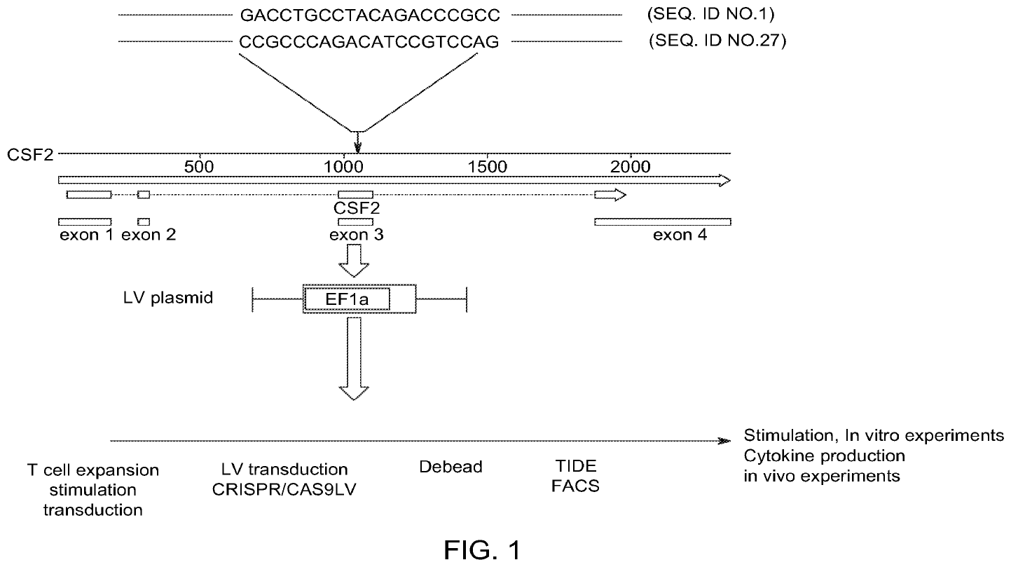

In some cases, a clustered regularly interspaced short palindromic repeat (CRISPR)/Cas system can be used (e.g., can be introduced into one or more T cells) to KO a nucleic acid encoding cytokine polypeptide (e.g., a GM-CSF polypeptide) (see, e.g., FIG. 1 and Example 1). A CRISPR/Cas system used to KO a nucleic acid encoding a cytokine polypeptide (e.g., a GM-CSF polypeptide) can include any appropriate guide RNA (gRNA). In some cases, a gRNA can be complementary to a nucleic acid encoding a GM-CSF polypeptide (e.g., a GM-CSF mRNA). Examples of gRNAs that are specific to a nucleic acid encoding a GM-CSF polypeptide include, without limitation, GACCTGCCTACAGACCCGCC (SEQ ID NO:1), GCAGTGCTGCTTGTAGTGGC (SEQ ID NO:10), TCAGGAGACGCCGGGCCTCC (SEQ ID NO:3), CAGCAGCAGTGTCTCTACTC (SEQ ID NO:4), CTCAGAAATGTTTGACCTCC (SEQ ID NO:5), and GGCCGGTCTCACTCCTGGAC (SEQ ID NO:6). In some cases, a gRNA component of a CRISPR/Cas system designed to KO a nucleic acid encoding a GM-CSF polypeptide can include the nucleic acid sequence set forth in SEQ ID NO:1.

A CRISPR/Cas system used to KO a nucleic acid encoding a cytokine polypeptide (e.g., a GM-CSF polypeptide) can include any appropriate Cas nuclease. Examples of Cas nucleases include, without limitation, Casl, Cas2, Cas3, Cas9, Cas10, and Cpfl. In some cases, a Cas component of a CRISPR/Cas system designed to KO a nucleic acid encoding a cytokine polypeptide (e.g., a GM-CSF polypeptide) can be a Cas9 nuclease. For example, the Cas9 nuclease of a CRISPR/Cas9 system described herein can be a lentiCRISPRv2 (see, e.g., Shalem et al., 2014 Science 343:84-87; and Sanjana et al., 2014 Nature methods 11: 783-784, each of which is incorporated herein by reference in its entirety).

Components of a CRISPR/Cas system (e.g., a gRNA and a Cas nuclease) used to KO a nucleic acid encoding a cytokine polypeptide (e.g., a GM-CSF polypeptide) can be introduced into one or more T cells (e.g., CARTs) in any appropriate format. In some cases, a component of a CRISPR/Cas system can be introduced into one or more T cells as a nucleic acid encoding a gRNA and/or a nucleic acid encoding a Cas nuclease. For example, a nucleic acid encoding at least one gRNA (e.g., a gRNA sequence specific to a nucleic acid encoding a GM-CSF polypeptide) and a nucleic acid at least one Cas nuclease (e.g., a Cas9 nuclease) can be introduced into one or more T cells. In some cases, a component of a CRISPR/Cas system can be introduced into one or more T cells as a gRNA and/or as a Cas nuclease. For example, at least one gRNA (e.g., a gRNA sequence specific to a nucleic acid encoding a GM-CSF polypeptide) and at least one Cas nuclease (e.g., a Cas9 nuclease) can be introduced into one or more T cells.

In some cases, when components of a CRISPR/Cas system (e.g., a gRNA and a Cas nuclease) are introduced into one or more T cells as nucleic acid encoding the components (e.g., nucleic acid encoding a gRNA and nucleic acid encoding a Cas nuclease), the nucleic acid can be any appropriate form. For example, a nucleic acid can be a construct (e.g., an expression construct). A nucleic acid encoding at least one gRNA and a nucleic acid encoding at least one Cas nuclease can be on separate nucleic acid constructs or on the same nucleic acid construct. In some cases, a nucleic acid encoding at least one gRNA and a nucleic acid encoding at least one Cas nuclease can be on a single nucleic acid construct. A nucleic acid construct can be any appropriate type of nucleic acid construct. Examples of nucleic acid constructs that can be used to express at least one gRNA and/or at least one Cas nuclease include, without limitation, expression plasmids and viral vectors (e.g., lentiviral vectors). In cases where a nucleic acid encoding at least one gRNA and a nucleic acid encoding at least one Cas nuclease are on separate nucleic acid constructs, the nucleic acid constructs can be the same type of construct or different types of constructs. In some cases, a nucleic acid encoding at least one gRNA sequence specific to a nucleic acid encoding a cytokine polypeptide (e.g., a GM-CSF polypeptide) and a nucleic acid encoding at least one Cas nuclease can be on a single lentiviral vector. For example, a lentiviral vector encoding at least one gRNA sequence specific to a nucleic acid encoding a cytokine polypeptide (e.g., GM-CSF polypeptide), encoding at least one gRNA including the sequence set forth in SEQ ID NO:1, and encoding at least one Cas9 nuclease can be used in ex vivo engineering of T cells to have a reduced expression level of that cytokine (e.g., a GM-CSF polypeptide).

In some cases, components of a CRISPR/Cas system (e.g., a gRNA and a Cas nuclease) can be introduced directly into one or more T cells (e.g., as a gRNA and/or as Cas nuclease). A gRNA and a Cas nuclease can be introduced into the one or more T cells separately or together. In cases where a gRNA and a Cas nuclease are introduced into the one or more T cells together, the gRNA and the Cas nuclease can be in a complex. When a gRNA and a Cas nuclease are in a complex, the gRNA and the Cas nuclease can be covalently or non-covalently attached. In some cases, a complex including a gRNA and a Cas nuclease also can include one or more additional components. Examples of complexes that can include components of a CRISPR/Cas system (e.g., a gRNA and a Cas nuclease) include, without limitation, ribonucleoproteins (RNPs) and effector complexes (e.g., containing a CRISPR RNAs (crRNAs) a Cas nuclease). For example, at least one gRNA and at least one Cas nuclease can be included in a RNP. In some cases, a RNP can include gRNAs and Cas nucleases at a ratio of about 1:1 to about 10:1 (e.g., about 1:1 to about 10:1, about 2:1 to about 10:1, about 3:1 to about 10:1, about 5:1 to about 10:1, about 8:1 to about 10:1, about 1:1 to about 9:1, about 1:1 to about 7:1, about 1:1 to about 5:1, about 1:1 to about 4:1, about 1:1 to about 3:1, about 1:1 to about 2:1, about 2:1 to about 8:1, about 3:1 to about 6:1, about 4:1 to about 5:1, or about 5:1 to about 7:1). For example, a RNP can include gRNAs and Cas nucleases at about a 1:1 ratio. For example, a RNP can include gRNAs and Cas nucleases at about a 2:1 ratio. In some cases, a RNP including at least one gRNA sequence specific to a nucleic acid encoding a GM-CSF polypeptide (e.g., encoding at least one gRNA including the sequence set forth in SEQ ID NO:1) and at least one Cas9 nuclease can be used in ex vivo engineering of T cells to have a reduced level of GM-CSF polypeptides.

Components of a CRISPR/Cas system (e.g., a gRNA and a Cas nuclease) used to KO a nucleic acid encoding a cytokine polypeptide (e.g., a GM-CSF polypeptide) can be introduced into one or more T cells (e.g., CARTs) using any appropriate method. A method of introducing components of a CRISPR/Cas system into a T cell can be a physical method. A method of introducing components of a CRISPR/Cas system into a T cell can be a chemical method. A method of introducing components of a CRISPR/Cas system into a T cell can be a particle-based method. Examples of methods that can be used to introduce components of a CRISPR/Cas system into one or more T cells include, without limitation, electroporation, transfection (e.g., lipofection), transduction (e.g., viral vector mediated transduction), microinjection, and nucleofection. In some cases, when components of a CRISPR/Cas system are introduced into one or more T cells as nucleic acid encoding the components, the nucleic acid encoding the components can be transduced into the one or more T cells. For example, a lentiviral vector encoding at least one gRNA sequence specific to a nucleic acid encoding a GM-CSF polypeptide (e.g., encoding at least one gRNA including the sequence set forth in SEQ ID NO:1) and at least one Cas9 nuclease can be transduced into T cells (e.g., ex vivo T cells). In some cases, when components of a CRISPR/Cas system are introduced directly into one or more T cells, the components can be electroporated into the one or more T cells. For example, a RNP including at least one gRNA sequence specific to a nucleic acid encoding a GM-CSF polypeptide (e.g., encoding at least one gRNA including the sequence set forth in SEQ ID NO:1) and at least one Cas9 nuclease can be electroporated into T cells (e.g., ex vivo T cells). In some cases, components of a CRISPR/Cas system can be introduced ex vivo into one or more T cells. For example, ex vivo engineering of T cells have a reduced level of GM-CSF polypeptides can include transducing isolated T cells with a lentiviral vector encoding components of a CRISPR/Cas system. For example, ex vivo engineering of T cells having reduced levels of GM-CSF polypeptides can include electroporating isolated T cells with a complex including components of a CRISPR/Cas system. In cases where T cells are engineered ex vivo to have a reduced level of GM-CSF polypeptides, the T cells can be obtained from any appropriate source (e.g., a mammal such as the mammal to be treated or a donor mammal, or a cell line).

In some cases, a T cell (e.g., a CART) can be treated with one or more inhibitors of GM-CSF polypeptide expression or GM-CSF polypeptide activity to reduce GM-CSF polypeptide expression in that T cell (e.g., as compared to a T cell that was not treated with one or more inhibitors of GM-CSF polypeptide expression or GM-CSF polypeptide activity). An inhibitor of GM-CSF polypeptide expression or GM-CSF polypeptide activity can be any appropriate inhibitor. Example of inhibitors of GM-CSF polypeptide expression or GM-CSF polypeptide activity include, without limitation, nucleic acid molecules designed to induce RNA interference (e.g., a siRNA molecule or a shRNA molecule), antisense molecules, miRNAs, receptor blockade, and antibodies (e.g., antagonistic antibodies and neutralizing antibodies).

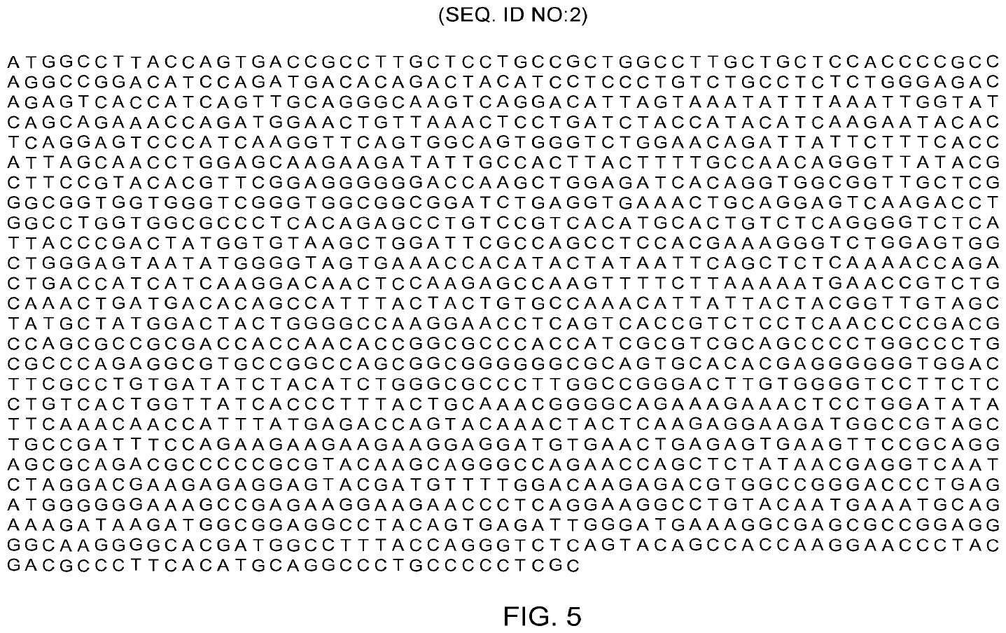

A T cell having (e.g., engineered to have) a reduced expression level of one or more cytokines (e.g., a GM-CSF KO T cell) can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, a T cell can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor expressed in a T cell having reduced expression of a cytokine polypeptide (e.g., a GM-CSF polypeptide) as described herein include, without limitation, cluster of differentiation 19 (CD19), mucin 1 (MUC-1), human epidermal growth factor receptor 2 (HER-2), estrogen receptor (ER), epidermal growth factor receptor (EGFR), alpha-fetoprotein (AFP), carcinoembryonic antigen (CEA), CA-125, epithelial tumor antigen (ETA), melanoma-associated antigen (MAGE), CD33, CD123, CLL-1, E-Cadherin, folate receptor alpha, folate receptor beta, IL13R, EGFRviii, CD22, CD20, kappa light chain, lambda light chain, desmopressin, CD44v, CD45, CD30, CD5, CD7, CD2, CD38, BCMA, CD138, FAP, CS-1, EphA3, EphA2, and C-met. For example, a T cell having a reduced level of GM-CSF polypeptides can be designed to express an antigen receptor targeting CD19. An exemplary nucleic acid sequence encoding a CAR targeting CD19 (CAR19) is shown in FIG. 5.

Any appropriate method can be used to express an antigen receptor on a T cell having (e.g., engineered to have) a reduced expression level of one or more cytokine polypeptides (e.g., a GM-CSF KO T cell). For example, a nucleic acid encoding an antigen receptor can be introduced into one or more T cells. In some cases, viral transduction can be used to introduce a nucleic acid encoding an antigen receptor into a non-dividing a cell. A nucleic acid encoding an antigen receptor can be introduced in a T cell using any appropriate method. In some cases, a nucleic acid encoding an antigen receptor can be introduced into a T cell by transduction (e.g., viral transduction using a retroviral vector such as a lentiviral vector) or transfection. In some cases, a nucleic acid encoding an antigen receptor can be introduced ex vivo into one or more T cells. For example, ex vivo engineering of T cells expressing an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line).

In some cases, when a T cell having (e.g., engineered to have) a reduced expression level of one or more cytokine polypeptides (e.g., a GM-CSF gene KO T cell) also expresses (e.g., is engineered to express) an antigen receptor, that T cell can be engineered to have a reduced expression level of that cytokine and engineered to express an antigen receptor using any appropriate method. In some cases, a T cell can be engineered to have a reduced expression level of a cytokine polypeptide (e.g., a GM-CSF polypeptide) first and engineered to express an antigen receptor second, or vice versa. In some cases, a T cell can be simultaneously engineered to have a reduced expression level of one or more cytokine polypeptides (e.g., a GM-CSF polypeptide) and to express an antigen receptor. For example, one or more nucleic acids used to reduce expression of a cytokine polypeptide such as a GM-CSF polypeptide (e.g., a lentiviral vector encoding at least one gRNA sequence specific to a nucleic acid encoding that cytokine and at least one Cas9 nuclease or a nucleic acid encoding at least one oligonucleotide that is complementary to that cytokine's mRNA) and one or more nucleic acids encoding an antigen receptor (e.g., a CAR) can be simultaneously introduced into one or more T cells. One or more nucleic acids used to reduce expression of a cytokine polypeptide (e.g., a GM-CSF polypeptide) and one or more nucleic acids encoding an antigen receptor can be introduced into one or more T cells on separate nucleic acid constructs or on a single nucleic acid construct. In some cases, one or more nucleic acids used to reduce expression of a cytokine polypeptide (e.g., a GM-CSF polypeptide) and one or more nucleic acids encoding an antigen receptor can be introduced into one or more T cells on a single nucleic acid construct. In some cases, one or more nucleic acids used to reduce expression of a cytokine polypeptide (e.g., a GM-CSF polypeptide) and one or more nucleic acids encoding an antigen receptor can be introduced ex vivo into one or more T cells. In cases where T cells are engineered ex vivo to have a reduced expression levels of one or more cytokine polypeptides (e.g., a GM-CSF polypeptide) and to express an antigen receptor, the T cells can be obtained from any appropriate source (e.g., a mammal such as the mammal to be treated or a donor mammal, or a cell line).

In some cases, a T cell having (e.g., engineered to have) a reduced expression level of one or more cytokine polypeptides (e.g., a GM-CSF KO T cell) can be stimulated. A T cell can be stimulated at the same time as being engineered to have a reduced level of one or more cytokine polypeptides or independently of being engineered to have a reduced level of one or more cytokine polypeptides. For example, one or more T cells having a reduced level of GM-CSF polypeptides used in an adoptive cell therapy can be stimulated first, and can be engineered to have a reduced expression level of GM-CSF polypeptides second, or vice versa. In some cases, one or more T cells having a reduced expression level of a cytokine polypeptide (e.g., a GM-CSF polypeptide) used in an adoptive cell therapy can be stimulated first, and can be engineered to have a reduced level of that cytokine polypeptide second. A T cell can be stimulated using any appropriate method. For example, a T cell can be stimulated by contacting the T cell with one or more CD polypeptides. Examples of CD polypeptides that can be used to stimulate a T cell include, without limitation, CD3, CD28, inducible T cell co-stimulator (ICOS), CD137, CD2, OX40, and CD27. In some cases, a T cell can be stimulated with CD3 and CD28 prior to introducing components of a CRISPR/Cas system (e.g., a gRNA and/or a Cas nuclease) to the T cell to KO a nucleic acid encoding one or more cytokine polypeptides (e.g., a GM-CSF polypeptide).

This document also provides methods and materials involved in treating cancer. For example, one or more T cells having (e.g., engineered to have) a reduced expression level of a cytokine polypeptide (e.g., a GM-CSF KO T cells) can be administered (e.g., in an adoptive cell therapy such as a CART therapy) to a mammal (e.g., a human) having cancer to treat the mammal. In some cases, methods of treating a mammal having cancer as described herein can reduce the number of cancer cells (e.g., cancer cells expressing a tumor antigen) within a mammal. In some cases, methods of treating a mammal having cancer as described herein can reduce the size of one or more tumors (e.g., tumors expressing a tumor antigen) within a mammal.

In some cases, administering T cells having (e.g., engineered to have) a reduced expression level of a cytokine polypeptide (e.g., a GM-CSF KO T cell) to a mammal does not result in CRS. For example, administering T cells having a reduced level of GM-CSF polypeptides to a mammal does not result in release of cytokines associated with CRS (e.g., CRS critical cytokines). Examples of cytokines associated with CRS include, without limitation, IL-6, G-CSF, IFN-g, IL-1B, IL-10, MCP-1, MIG, MIP, MIP lb, TNF-a, IL-2, and perforin.

In some cases, administering T cells having (e.g., engineered to have) a reduced expression level of a cytokine polypeptide (e.g., a GM-CSF KO T cell) to a mammal does not result in neurotoxicity. For example, administering T cells having a reduced level of GM-CSF polypeptides to a mammal does not result in differentiation and/or activation of white blood cells, the differentiation and/or activation of which, is associated with neurotoxicity. Examples of white blood cells, the differentiation and/or activation of which, is associated with neurotoxicity include, without limitation, monocytes, macrophages, T-cells, dendritic cells, microglia, astrocytes, and neutrophils.

Any appropriate mammal (e.g., a human) having a cancer can be treated as described herein. Examples of mammals that can be treated as described herein include, without limitation, humans, primates (such as monkeys), dogs, cats, horses, cows, pigs, sheep, mice, and rats. For example, a human having a cancer can be treated with one or more T cells having (e.g., engineered to have) a reduced expression level of a cytokine polypeptide (e.g., a GM-CSF polypeptide) in, for example, an adoptive T cell therapy such as a CART cell therapy using the methods and materials described herein.

When treating a mammal (e.g., a human) having a cancer as described herein, the cancer can be any appropriate cancer. In some cases, a cancer treated as described herein can be a solid tumor. In some cases, a cancer treated as described herein can be a hematological cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a tumor-associated antigen (e.g., an antigenic substance produced by a cancer cell). Examples of cancers that can be treated as described herein include, without limitation, B cell cancers (e.g., diffuse large B cell lymphoma (DLBCL) and B cell leukemias), acute lymphoblastic leukemia (ALL), chronic lymphocytic leukemia (CLL), follicular lymphoma, mantle cell lymphoma, non-Hodgkin lymphoma, Hodgkin lymphoma, acute myeloid leukemia (AML), multiple myeloma, head and neck cancers, sarcomas, breast cancer, gastrointestinal malignancies, bladder cancers, urothelial cancers, kidney cancers, lung cancers, prostate cancers, ovarian cancers, cervical cancers, genital cancers (e.g., male genital cancers and female genital cancers), and bone cancers. For example, one or more T cells having (e.g., engineered to have) a reduced level of GM-CSF polypeptides (e.g., a GM-CSF KO T cells) can be used to treat a mammal having DLBCL. For example, one or more T cells having (e.g., engineered to have) a reduced level of GM-CSF polypeptides (e.g., a GM-CSF KO T cells) can be used to treat a mammal having ALL.

Any appropriate method can be used to identify a mammal having cancer. For example, imaging techniques and biopsy techniques can be used to identify mammals (e.g., humans) having cancer.

Once identified as having a cancer (e.g., DLBCL or ALL), a mammal can be administered one or more T cells having (e.g., engineered to have) a reduced expression level of a cytokine polypeptide (e.g., a GM-CSF KO T cells) described herein.

For example, one or more T cells having (e.g., engineered to have) a reduced expression level of a cytokine polypeptide (e.g., a GM-CSF KO T cells) can be used in an adoptive T cell therapy (e.g., a CART cell therapy) to treat a mammal having a cancer. For example, one or more T cells having a reduced level of GM-CSF polypeptides can be used in an adoptive T cell therapy (e.g., a CART cell therapy) targeting any appropriate antigen within a mammal (e.g., a mammal having cancer). In some cases, an antigen can be a tumor-associated antigen (e.g., an antigenic substance produced by a cancer cell). Examples of tumor-associated antigens that can be targeted by an adoptive T cell therapy provided herein include, without limitation, CD19 (associated with DLBCL, ALL, FL, MCL, and CLL), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33 (associated with AML), CD123 (associated with AML), CLL-1 (associated with AML), E-Cadherin (associated with epithelial tumors), folate receptor alpha (associated with ovarian cancers), folate receptor feta (associated with ovarian cancers and AML), IL13R (associated with brain cancers), EGFRviii (associated with brain cancers), CD22 (associated with B cell cancers), CD20 (associated with B cell cancers), kappa light chain (associated with B cell cancers), lambda light chain (associated with B cell cancers), CD44v (associated with AML), CD45 (associated with hematological cancers), CD30 (associated with Hodgkin lymphomas and T cell lymphomas), CD5 (associated with T cell lymphomas), CD7 (associated with T cell lymphomas), CD2 (associated with T cell lymphomas), CD38 (associated with multiple myelomas and AML), BCMA (associated with multiple myelomas), CD138 (associated with multiple myelomas and AML), FAP (associated with solid tumors), CS-1 (associated with multiple myeloma), EphA3 (associated with solid tumors including breast, lung, colon, prostate, renal, glioblastoma multiforme, and melanoma) and c-Met (associated with breast cancer). For example, one or more T cells having a reduced level of GM-CSF polypeptides can be used in CART cell therapy targeting CD19 (e.g., CART19 cell therapy) to treat cancer as described herein.

In some cases, one or more T cells having (e.g., engineered to have) a reduced expression level of a cytokine polypeptide (e.g., a GM-CSF KO T cells) can be used in an adoptive T cell therapy (e.g., a CART cell therapy) to treat a mammal having a disease or disorder other than cancer. For example, one or more T cells having a reduced level of GM-CSF polypeptides can be used in an adoptive T cell therapy (e.g., a CART cell therapy) targeting any appropriate disease-associated antigen (e.g., an antigenic substance produced by cell affected by a particular disease) within a mammal. Examples of disease-associated antigens that can be targeted by an adoptive T cell therapy provided herein include, without limitation desmopressin (associated with auto immune skin diseases). In another embodiment, the disease-associated antigens that can be targeted by an adoptive T cell therapy provided herein include, but are not limited to the DSG3 antigen, the B cell receptor (BCR) that binds to DSG3 in Pemphigus Vulgaris or the antigen MuSK. In a further embodiment, the disease-associated antigens that can be targeted by an adoptive T cell therapy provided herein include, but are not limited to the BCR for MuSK in MuSK Myasthenia Gravis.

In some cases, one or more T cells having (e.g., engineered to have) a reduced expression level of a cytokine polypeptide (e.g., a GM-CSF KO T cells) used in an adoptive T cell therapy (e.g., a CART cell therapy) can be administered to a mammal having a cancer as a combination therapy with one or more additional agents used to treat a cancer. For example, one or more T cells having a reduced level of GM-CSF polypeptides used in an adoptive cell therapy can be administered to a mammal in combination with one or more anti-cancer treatments (e.g., surgery, radiation therapy, chemotherapy (e.g., alkylating agents such as busulfan), targeted therapies (e.g., GM-CSF inhibiting agents such as lenzilumab), hormonal therapy, angiogenesis inhibitors, immunosuppressants (e.g., interleukin-6 inhibiting agents such as tocilizumab)) and/or one or more CRS treatments (e.g., ruxolitinib and ibrutinib). In cases where one or more T cells having a reduced level of GM-CSF polypeptides used in an adoptive cell therapy are used with additional agents treat a cancer, the one or more additional agents can be administered at the same time or independently. In some cases, one or more T cells having a reduced level of GM-CSF polypeptides used in an adoptive cell therapy can be administered first, and the one or more additional agents administered second, or vice versa.

Lenzilumab (Humanigen, Burlingame, CA), an hGM-CSF neutralizing antibody in accordance with embodiments described herein and as described in U.S. Patent Nos. 8,168,183 and 9,017, 674, each of which is incorporated herein by reference in its entirety, is a novel, first in class Humaneered® monoclonal antibody that neutralizes human GM-CSF.

All scientific publications cited herein are hereby incorporated by reference in their entireties.

The following examples are presented in order to illustrate certain embodiments of the invention more fully. The examples should in no way be construed, however, as limiting the broad scope of the invention described in the claims.

### EXAMPLES

### Example 1

### Generation of Cytokine to Deficient CART Cells to Increase Therapeutic Index of CART Cell Therapy

This example describes the development of GM-CSF knocked out (GM-CSF KO) CART19 cells, and shows that the resulting GM-CSF KO CART19 cells function normally and have enhanced expansion.

### Experimental Design:

CAR19 in B cell leukemia xenografts were used. These plasmids were used for packaging and lentivirus production as described herein. As a mouse model, two models were employed:

1. Xenograft models: NSG mice were subcutaneously engrafted with the CD19 positive, luciferase positive cell line NALM6. Engraftment was confirmed by bioluminescence imaging. Mice were treated with human PBMCs intravenously and intra-tumor injection of lentivirus particles. Generation of CART cells is measured by flow cytometry. Trafficking of CARTs to tumor sites is assessed and anti-tumor response is measured by bioluminescence imaging as a measure of disease burden.
2. Humanized Immune System (HIS) mice from the Jackson Laboratory: These mice were injected with fetal CD34+ cells as neonates and therefore develop human hematopoiesis. We will engraft these mice with the CD19+ cell line NALM6, as previously used. Similarly, we will generate CART19 in vivo through the intratumoral injection of lentivirus particles. Then will measure the activity of CART19 cells in eradication of NALM6 and compare that to ex vivo generated lenti-virally transduced CART 19 cells (currently used in the clinic).

### Materials and Methods:

### Generation of CAR Plasmid::

The anti-CD19 clone FMC63 was do novo synthesized into a CAR backbone using 41BB and CD3 zeta and then cloned into a third generation lentivirus backbone.

To generate the control CART19 cells, normal donor T cells were negatively selected using pan T cell kit and expanded ex vivo using anti-CD3/CD28 Dynabeads (Invitrogen, added on the first day of culture). T cells were transduced with lentiviral supernatant one day following stimulation at a multiplicity of infection (MOI) of 3. The anti-CD3/CD28 Dynabeads were removed on day 6 and T cells were grown in T cell media (X-vivo 15 media, human serum 5%, penicillin, streptomycin and glutamine) for up to 15 days and then cryopreserved for future experiments. Prior to all experiments, T cells were thawed and rested overnight at 37° C.

### Generation of GM-CSF Knock Out CART Cells:

GM-CSF knockout CART cells were generated with a CRISR-Cas9 system, using two methodologies:

1. gRNA was generated and cloned into a lentivirus vector that encodes Cas9 and the gRNA. During T cell expansion, T cells were transduced with this lentivirus on Day 1, on the same day and simultaneously with CAR19 lentivirus particles. Cells were expanded for a period of 8 days and then T cell were harvested, DNA isolated and sequenced to assess the efficiency of knockout. These cells were cryopreserved and used for future in vitro or in vivo experiments. A nucleic acid sequence encoding is shown in FIG. 5.
2. mRNA was generated from the gRNA and used it to knock out GM-CSF. To do so, gRNA was mixed with RNP at 1:1 ratio and then T cells were electroporated on Day 3 post stimulation with CD3/CD28 beads. Cells were expanded for a period of 8 days and then T cell were harvested, DNA isolated and sequenced to assess the efficiency of knockout. These cells were cryopreserved and used for future in vitro or in vivo experiments

### Cells

The NALM6 cell line was obtained from the ATCC and maintained in R10 media (RPMI media, 10% fetal calf serum, penicillin, and streptomycin). NALM6-cells transduced with luciferase-GFP cells under the control of the EF 1 a promoter were used in some experiments as indicated. De-identified primary human ALL specimens were obtained from the Mayo Clinic Biobank. All samples were obtained after informed, written consent. For all functional studies, cells were thawed at least 12 hours before analysis and rested overnight at 37° C.

### Flow Cytometry Analysis

Anti-human antibodies were purchased from BioLegend, eBioscience, or BD Biosciences. Cells were isolated from in vitro culture or from animals, washed once in PBS supplemented with 2% fetal calf serum, and stained at 4° C. after blockade of Fc receptors. For cell number quantitation, Countbright beads (Invitrogen) were used according to the manufacturer's instructions (Invitrogen). In all analyses, the population of interest was gated based on forward vs. side scatter characteristics followed by singlet gating, and live cells were gated using Live Dead Aqua (Invitrogen). Surface expression of anti-CD19 CAR was detected by staining with an Alexa Fluor 647-conjugated goat anti-mouse F(ab′)2 antibody from Jackson Immunoresearch.

### T Cell Function Assays:

### T Cell Degranulation and Intracellular Cytokine Assays:

Briefly, T cells were incubated with target cells at a 1:5 ratio. After staining for CAR expression; CD107a, CD28, CD49d and monensin were added at the time of incubation. After 4 hours, cells were harvested and stained for CAR expression, CD3 and Live Dead staining (Invitrogen). Cells were fixed and permeabilized (FIX & PERM® Cell Fixation & Cell Permeabilization Kit, Life technologies) and intracellular cytokine staining was then performed.

### Proliferation Assays:

T cells were washed and resuspended at 1×10⁷/ml in 100 μl of PBS and labeled with 100 μl of CFSE 2.50/1 (Life Technologies) for 5 minutes at 37° C. The reaction was then quenched with cold R10, and the cells were washed three times. Targets were irradiated at a dose of 100 Gy. T cells were incubated at a 1:1 ratio with irradiated target cells for 120 hours. Cells were then harvested, stained for CD3, CAR and Live Dead aqua (Invitrogen), and Countbright beads (Invitrogen) were added prior to flow cytometric analysis.

### Cytotoxicity Assays:

NALM6-Luc cells or CFSE (Invitrogen) labelled primary ALL samples were used for cytotoxicity assay. In brief, targets were incubated at the indicated ratios with effector T cells for 4, 16, 24, 48, and/or 72 hours. Killing was calculated either by bioluminescence imaging on a Xenogen IVIS-200 Spectrum camera or by flow cytometry. For the latter, cells were harvested; Countbright beads and 7-AAD (Invitrogen) were added prior to analysis. Residual live target cells were CFSE+ 7-AAD−.

### Secreted Cytokine Measurement:

Effector and target cells were incubated at a 1:1 ratio in T cell media for 24 or 72 hours as indicated. Supernatant was harvested and analyzed by 30-plex Luminex array according to the manufacturer's protocol (Invitrogen).

### Results

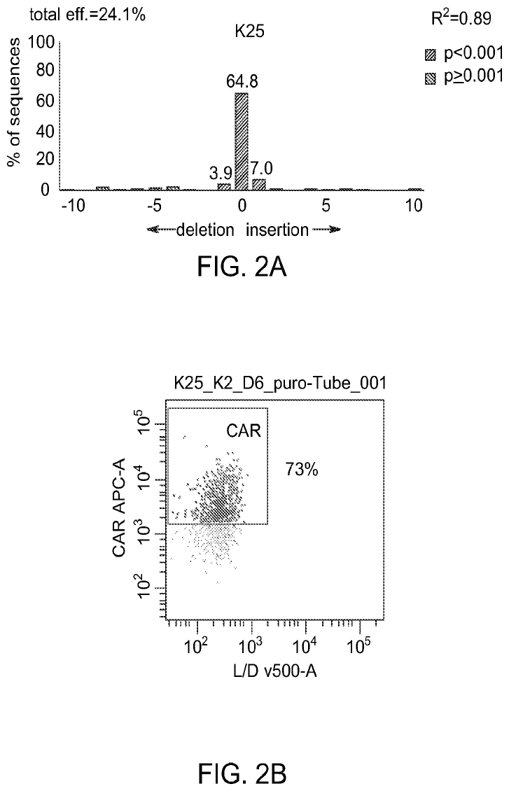

GM-CSF KO CART cells were generated with a CRISR-Cas9 system. During T cell expansion, T cells were transduced (Day 1) with lentivirus encoding gRNA and Cas9 and lentivirus encoding CARI 9. Cells were expanded for a period of 8 days. After 8 days, T cells were harvested, DNA was isolated, and the isolated DNA was sequenced to assess the efficiency of knockout. See, e.g., FIG. 1. T cells exhibited a knockout efficiency of 24.1% (FIG. 2A), and CAR transduction efficiency was 73% (FIG. 2B).

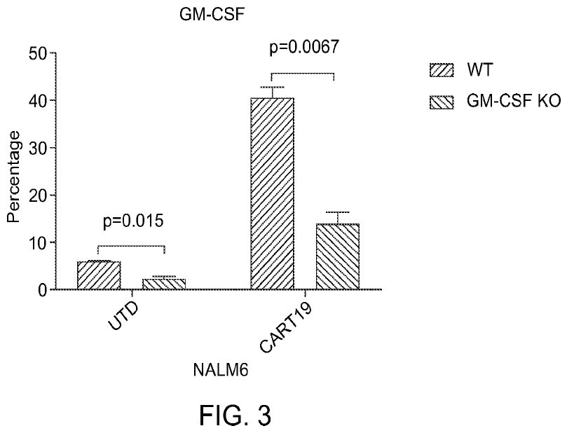

To evaluate cell effector functions of GM-CSF KO CART cells, CART19, GM-CSF KO CART19, UTD, or GM-CSF KO UTD were co-cultured with the CD19 positive cell line NALM6 at a ratio of 1:5. After 4 hours, the cells were harvested, permeabilized, fixed, and stained for cytokines (FIG. 3).

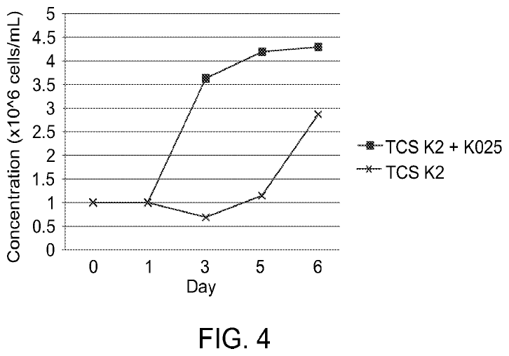

To evaluate proliferation of GM-CSF KO CART cells, expansion kinetics were followed after T cells were transduced. GM-CSF KO CART cells expand more robustly than cells transduced with CART19 alone (FIG. 4).

These results demonstrate that GM-CSF knockout CARTs can enhance CART cell function and antitumor activity. These results also demonstrate that blockade of GMCSF in combination with CART19 does not impact CART cell effector functions.

### Example 2

### GM-CSF Depletion During CART Therapy Reduces Cytokine Release Syndrome and Neurotoxicity and May Enhance CART Cell Function

This example investigates depleting granulocyte macrophage colony-stimulating factor (GM-CSF) and myeloid cells as a potential strategy to manage CART cell associated toxicities. It was found that the GM-CSF blockade with a neutralizing antibody does not inhibit CART function in vitro or in vivo. CART cell proliferation was enhanced in vitro, and CART cells resulted in a more efficient control of leukemia in patient derived xenografts after GM-CSF depletion. Furthermore, in a primary acute lymphoblastic leukemia xenograft model of CRS and NT, GM-CSF blockade resulted in a reduction of myeloid cell and T cell infiltration in the brain, and ameliorated the development of CRS and NT. Finally, GM-CSF knocked out CART cells were generated through CRISPR/cas9 disruption of GM-CSF during CART cell manufacturing. GM-CSF k r CART cells continued to function normally and had resulted in enhanced anti-tumor activity in vivo. These demonstrate that GM-CSF neutralization can abrogate neurotoxicity and CRS, and also can enhance CART cell functions.

### Materials and Methods

### Cells Lines and Primary Cells

NALM6 and MOLM13 were purchased from ATCC, Manassas, VA, USA, transduced with a luciferase-ZsGreen lentivirus (addgene) and sorted to 100% purity. Cell lined were cultured in RIO (RPMI, 10% FCS v/v, 1% pen strep v/v). Primary cells were obtained from the Mayo Clinic biobank for patients with acute leukemia under an institutional review board approved protocol. The use of recombinant DNA in the laboratory was approved by the Institutional Biosafety Committee (IBC).

### Primary T Cells and CART Cells

Peripheral blood mononuclear cells (PBMC) were isolated from de-identified donor blood apheresis cones using a FICOLL protocol (see, e.g., Dietz et al., 2006 Transfusion 46:2083-2089, which is incorporated herein by reference in its entirety). T cells were separated with negative selection magnetic beads (Stemcell technologies) and monocytes were positively selected using CD14+ magnetic beads (Stemcell technologies). Primary cells were cultured in X-Vivo 15 media with 5% human serum, penicillin, streptomycin and glutamax. CD19 directed CART cells were generated through the lentiviral transduction of normal donor T cells as described below. Second generation CARI 9 constructs were do nova synthesized (IDT) and cloned into a third generation lentivirus under the control of EF-la promotor. The CD19 directed single chain variable fragment was derived from the clone FMC63. A second generation 41BB co-stimulated (FMC63-41BBz) CAR construct was synthesized and used for these experiments. Lentivirus particles were generated through the transient transfection of plasmid into 293T virus producing cells, in the presence of lipofectamine 3000, VSV-G and packaging plasmids. T cells isolated from normal donors were stimulated using CD3/CD28 stimulating beads (StemCell) at 1:3 ratio and then transduced with lentivirus particles 24 hours after stimulation at a multiplicity of infection of 3.0. Magnetic bead removal was performed on Day 6 and CART cells were harvested and cryopreserved on Day 8 for future experiments. CART cells were thawed and rested in T cell medium 12 hours prior to their use in experiments.

### Generation of GM-CSF^{k/o} CART Cells:

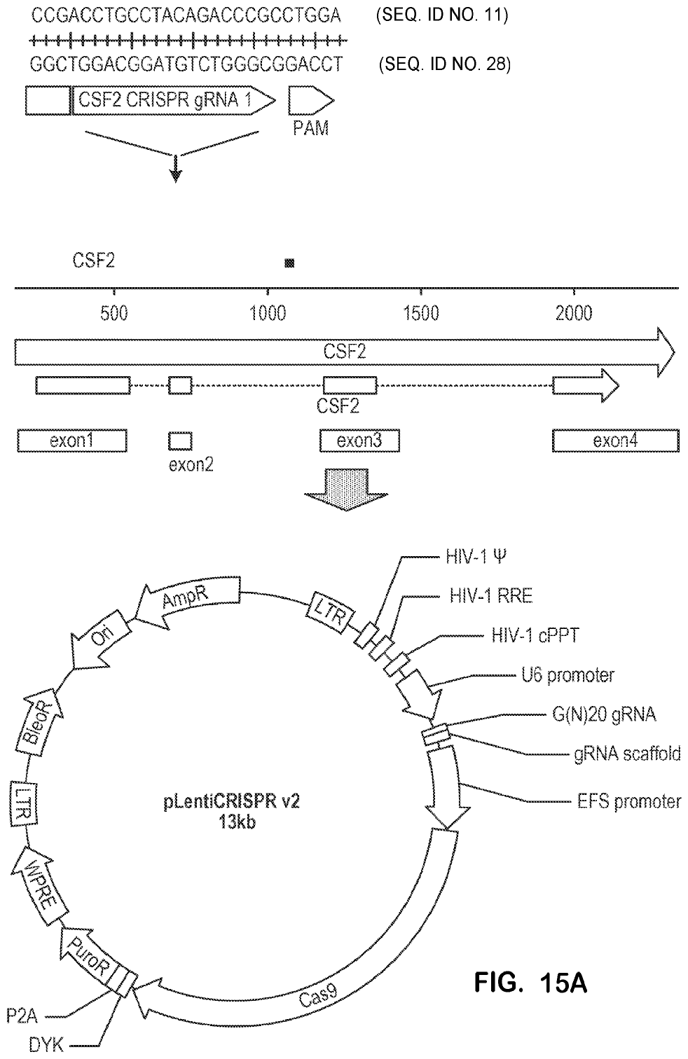

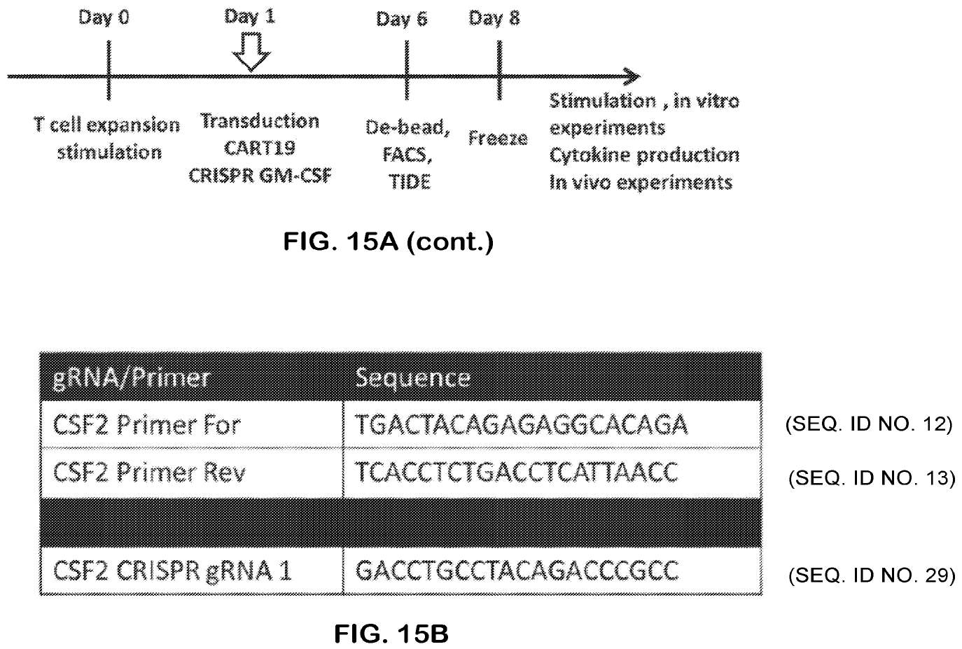

A guide RNA (gRNA) targeting exon 3 of human GM-CSF was selected via screening gRNAs previously reported to have high efficiency for human GM-CSF.25 This gRNA was ordered in a CAS9 third generation lentivirus construct (lentiCRISPRv2), controlled under a U6 promotor (GenScript, Township, NJ, USA). Lentiviral particles encoding this construct were produced as described above. T cells were dual transduced with CAR19 and GM-CSFgRNA-lentiCRISPRv2 lentiviruses, 24 hours after stimulation with CD3/CD28 beads. CAR-T cell expansion was then continued as described above. To analyze efficiency of targeting GM-CSF, genomic DNA was extracted from the GM-CSFk/o CART19 cells using PureLink Genomic DNA Mini Kit (Invitrogen, Carlsbad, CA, USA). The DNA of interest was PCR amplified using Choice Taq Blue Mastermix (Thomas Scientific, Minneapolis, MN, USA) and gel extracted using QIAquick Gel Extraction Kit (Qiagen, Germantown, MD, USA) to determine editing. PCR amplicons were sent for Eurofins sequencing (Louisville, KY, USA) and allele modification frequency was calculated using TIDE (Tracking of lndels by Decomposition) software available at tide.nki.nl. FIGS. 15A-15B describe the gRNA sequence, primer sequences, and the schema for generation of GM-CSFk/o CART 19 schema.

### GM-CSF Neutralizing Antibodies and Isotype Controls

Lenzilumab (Humanigen, Brisbane, CA) is a humanized antibody that neutralizes human GM-CSF, as described in U.S. Pat. Nos. 8,168,183 and 9,017, 674, each of which is incorporated herein by reference in its entirety. For in vitro experiments, lenzilumab or isotype control 10 ug/mL was used. For in vivo experiments, 10 mg/kg of lenzilumab or isotype control was injected, and the schedule, route and frequency are indicated in the individual experimental schema. In some experiments, anti-mouse GM-CSF neutralizing antibody (10 mg/kg) was also used, as indicated in the experimental schema.

### T Cell Functional Experiments

Cytokine assays were performed 24 or 72 hours after a co-culture of CART cells with their targets at 1:1 ratio as indicated. Human GM-CSF singleplex (Millipore), 30-plex human multiplex (Millipore), or 30-plex mouse multiplex (Millipore) was performed on supernatant collected from these experiments, as indicated. This was analyzed using flow cytometry bead assay or Luminex, Intracellular cytokine analysis and T cell degranulation assays were performed following incubation of CART cells with targets at 1:5 ratio for 4 hours at 37° C., in the presence of monensin, hCD49d, and hCD28. After 4 hours, cells were harvested and intracellular staining was performed after surface staining, followed by fixation and permealization (FIX & PERM Cell Fixation & Cell Permeabilization Kit, Life Technologies). For proliferation assays, CFSE (Life Technologies) labeled effector cells (CART19), and irradiated target cells were co cultured at 1:1. In some experiments with CD14+ monocytes was added to the co-culture at 1:1:1 ratio as indicated. Cells were co-cultured for 3-5 days, as indicated in the specific experiment and then cells were harvested and surface staining with anti-hCD3 and live/dead aqua was performed. PMA/ionomycin was used as a positive non-specific stimulant of T cells, at different concentrations as indicated in the specific experiments. For killing assays, the CD19+Luciferase+ ALL cell line NALM6 or the CD19⁻Luciferase⁺ control MOLM13 cells were incubated at the indicated ratios with effector T cells for 24 or 48 hours as listed in the specific experiment. Killing was calculated by bioluminescence imaging on a Xenogen IVIS-200 Spectrum camera (PerkinElmer, Hopkinton, MA, USA) as a measure of residual live cells. Samples were treated with 1 μl D-luciferin (30 ug/mL) per 100 μl sample volume, 10 minutes prior to imaging.

### Multi-parametric Flow Cytometry

Anti-human antibodies were purchased from Biolegend, eBioscience, or BD Biosciences. Cells were isolated from in vitro culture or from peripheral blood of animals (after ACK lysis), washed twice in phosphate-buffered saline supplemented with 2% fetal calf serum and stained at 4° C. For cell number quantitation, Countbright beads (Invitrogen) were used according to the manufacturer's instructions (Invitrogen). In all analyses, the population of interest was gated based on forward vs side scatter characteristics, followed by singlet gating, and live cells were gated using Live Dead Aqua (Invitrogen). Surface expression of CAR was detected by staining with a goat anti-mouse F(ab′)2 antibody. Flow cytometry was performed on a four-laser Canto II analyzer (BD Biosciences). All analyses were performed using FlowJo X10.0.7r2.

### Xenogeneic Mouse Models

Male and female 8-12-week old NOD-SCID-IL2ry−/− (NSG) mice were bred and cared for within the Department of Comparative Medicine at the Mayo Clinic under a breeding protocol approved by the Institutional Animal Care and Use Committee (IACUC). Mice were maintained in an animal barrier spaces that is approved by the institutional Biosafety Committee for BSL2+ level experiments.

### NALM6 Cell Line Xenografts

The CD19+, luciferase+ ALL NALM6 cell line was used to establish ALL xenografts. These xenograft experiments were approved by a different IACUC protocol. Here, 1×10⁶ cells were injected intravenously via a tail vein injection. After injection, mice underwent bioluminescent imaging using a Xenogen IVIS-200 Spectrum camera six days later, to confirm engraftment. Imaging was performed after the intraperitoneal injection of 10 Wig D luciferin (15 mg/ml). Mice were then randomized based on their bioluminescent imaging to receive different treatments as outlined in the specific experiments. Typically, 1-2×10⁶ CART cells or UTD cells are injected and exact doses are listed in the specific experimental details. Weekly imaging was performed to assess and follow disease burden. Tail vein bleeding was done 7-10 days after injection of CART cells to assess T cell expansion and as needed following that. Mouse peripheral blood was lysed using ACK lysing buffer (Thermofisher) and then used for flow cytometry studies. Bioluminescent images were acquired using a Xenogen IVIS-200 Spectrum camera (PerkinElmer, Hopkinton, MA, USA) and analyzed using Living Image version 4.4 (Caliper LifeSciences, PerkinElmer). For antibody treated mice, antibody therapy (10 mg/kg lenzilumab or isotype control) was commenced IP, for a total of 10 days.

### Primary Patient Derived ALL Xenografts

To establish primary ALL xenografts, NSG mice first received 30 mg/kg busulfan IP. The following day, mice were injected with 2×10⁶ primary blasts derived from the peripheral blood of patients with relapsed refractory ALL. Mice were monitored for engraftment for 4-6 weeks and when CD19+ cells were consistently observed in the blood (>1 ce11/0), they were randomized to receive different treatments of CART19 or UTD (1×10⁶ cells) with or without antibody therapy (10 mg/kg lenzilumab or isotype control IP for a total of 10 days, starting on the day they received CART cell therapy). Mice were periodically monitored for leukemic burden via tail vein bleeding.

### Primary Patient Derived ALL Xenografts for CRS/NT

Similar to the experiments above, mice were IP injected with 30 mg/kg busulfan. The following day, they received 1-2×10⁶ primary blasts derived from the peripheral blood of patients with relapsed refractory ALL. Mice were monitored for engraftment for 4-6 weeks and when CD19+ cell level was high (>10 cells/0), they received CART19 (2-5×10⁶ cells) and commenced antibody therapy for a total of 10 days, as indicated in the details of the specific experiment. Mice were weighed on daily basis as a measure of their well-being. Brian MRI of the mice was performed 5-6 days post CART injection and tail vein bleeding was performed 4-11 days post CART injection. Brain MRI images were analyzed using Azalyze.

### MRI Acquisition

A Bruker Avance II 7 Tesla vertical bore small animal MRI system (Bruker Biospin) was used for image acquisition to evaluate central nervous system (CNS) vascular permeability. Inhalation anesthesia was induced and maintained via 3 to 4% isoflurane. Respiratory rate was monitored during the acquisition sessions using an MRI compatible vital sign monitoring system (Model 1030; SA Instruments, Stony Brook, NY). Mice were given an IP injection of gadolinium using weight-based dosing of 100 mg/kg, and after a standard delay of 15 min, a volume acquisition T1-weighted spin echo sequence was used (repetition time=150 ms, echo time=8 ms, field of view: 32 mm×19.2 mm×19.2 mm, matrix: 160×96×96; number of averages=1) to obtain T1-weighted images. Gadolinium-enhanced MRI changes were indicative of blood-brain-barrier disruption. Volumetric analysis was performed using Analyze Software package developed by the Biomedical Imaging Resource at Mayo Clinic.

### RNA-Seq on Mouse Brain Tissue

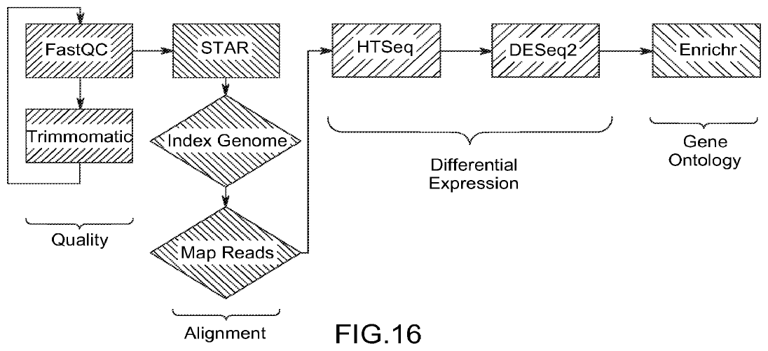

RNA was isolated using miRNeasy Micro kit (Qiagen, Gaithersburg, MD, USA) and treated with RNase-Free DNase Set (Qiagen, Gaithersburg, MD, USA). RNA-seq was performed on an Illumina HTSeq 4000 (Illumina, San Diego, CA, USA) by the Genome Analysis Core at Mayo Clinic. The binary base call data was converted to fastq using Illumina bcl2fastq software. The adapter sequences were removed using Trimmomatic, and FastQC was used to check for quality. The latest human (GRCh38) and mouse (GRCm38) reference genomes were downloaded from NCBI. Genome index files were generated using STAR, and the paired end reads were mapped to the genome for each condition. HTSeq3 1 was used to generate expression counts for each gene, and DeSeq2 was used to calculate differential expression. Gene ontology was assessed using Enrichr. FIG. 16 summarizes the steps detailed above. RNA sequencing data are available at the Gene Expression Omnibus under accession number GSE121591.

### Statistics

Prism Graph Pad and Microsoft Excel used to analyze data. The high cytokine concentrations in the heat map were normalized to “1” and low concentrations normalized to “0” via Prism. Statistical tests described in figure legends.

### Results

### GM-CSF Neutralization In Vitro Enhances CAR-T Cell Proliferation in the Presence of Monocytes and Does Not Impair CAR-T Cell Effector Function.

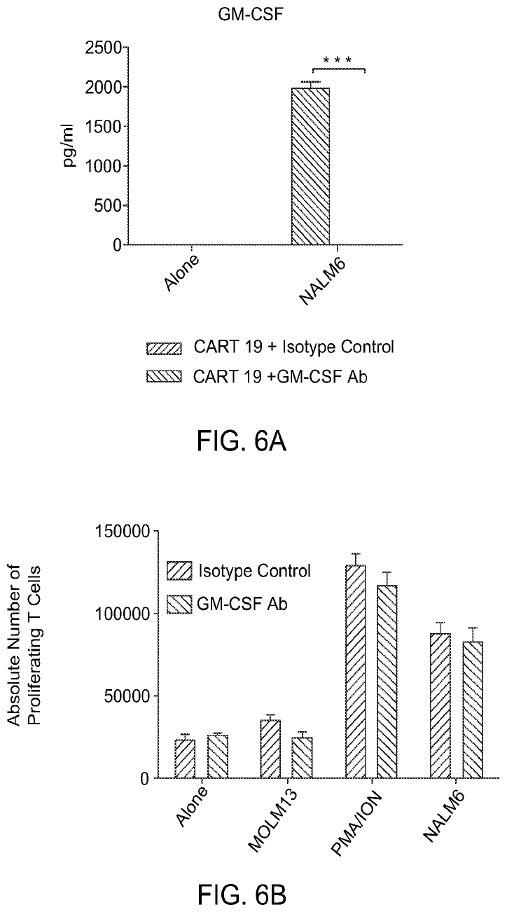

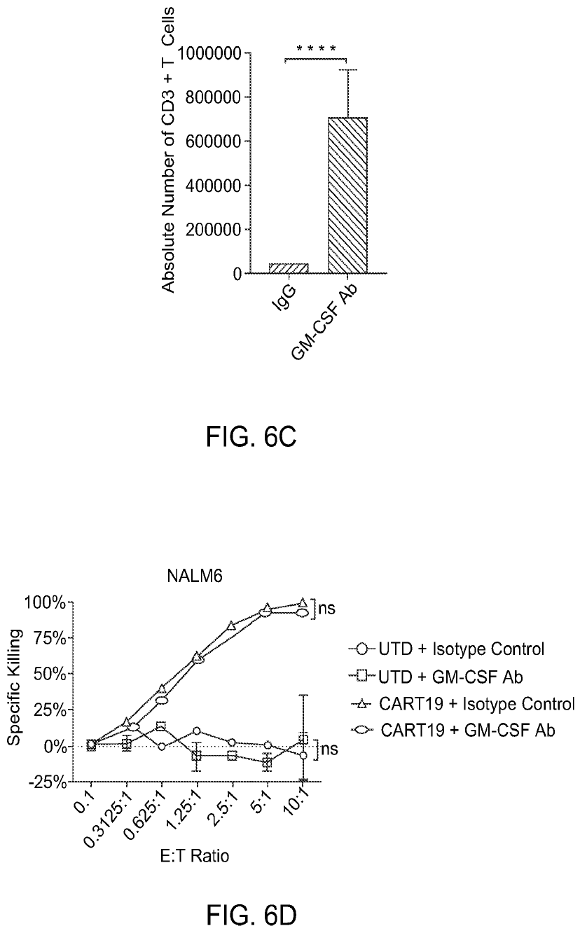

If GM-CSF neutralization after CAR-T cell therapy is to be utilized as a strategy to prevent CRS and NT, it must not inhibit CAR-T cell efficacy. Therefore, our initial experiments aimed to investigate the impact of GM-CSF neutralization on CAR-T cell effector functions. Here, CART19 cells were co-cultured with or without the CD19+ALL cell line NALM6 in the presence of lenzilumab (GM-CSF neutralizing antibody) or an isotype control (IgG). We established that lenzilumab, but not IgG control antibody, was indeed able to completely neutralize GM-CSF (FIG. 6A) but did not inhibit CAR-T cell antigen specific proliferation (FIG. 6B). When CART19 cells were co-cultured with the CD19+ cell line NALM6 in the presence of monocytes, lenzilumab in combination with CART19 demonstrated an exponential increase in antigen specific CART19 proliferation compared to CART19 plus isotype control IgG (P<0.0001, FIG. 6C). To investigate CAR-T specific cytotoxicity, either CART19 or control UTD T cells were cultured with the luciferase+CD19+ NALM6 cell line and treated with either isotype control antibody or GM-CSF neutralizing antibody (FIG. 6D). GM-CSF neutralizing antibody treatment did not inhibit the ability of CAR-T cells to kill NALM6 target cells (FIG. 6D). Overall, these results indicate that lenzilumab does not inhibit CAR-T cell function in vitro and enhances CART19 cell proliferation in the presence of monocytes, suggesting that GM-CSF neutralization may improve CAR-T cell mediated efficacy.

### GM-CSF Neutralization In Vivo Enhances CAR-T Cell Anti-tumor Activity in Xenograft Models.

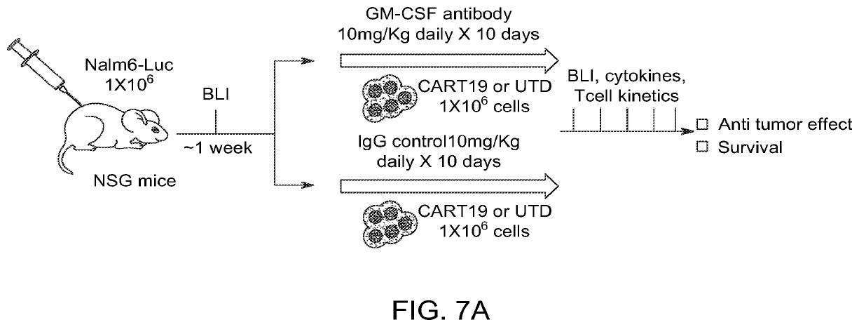

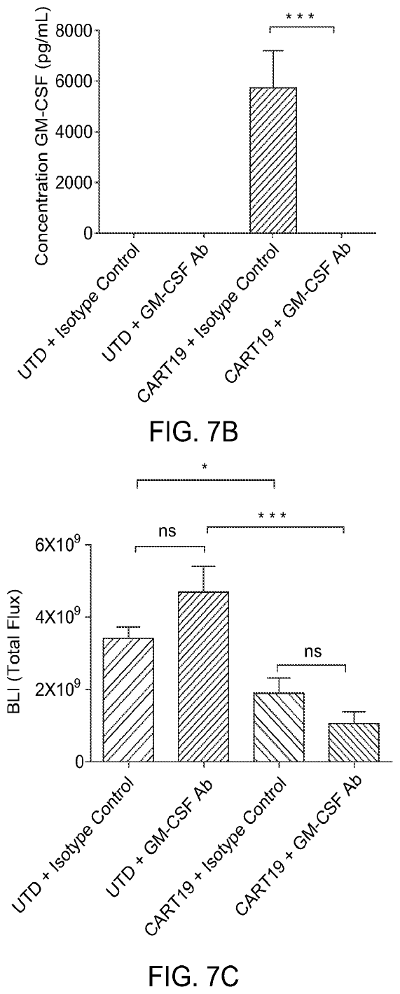

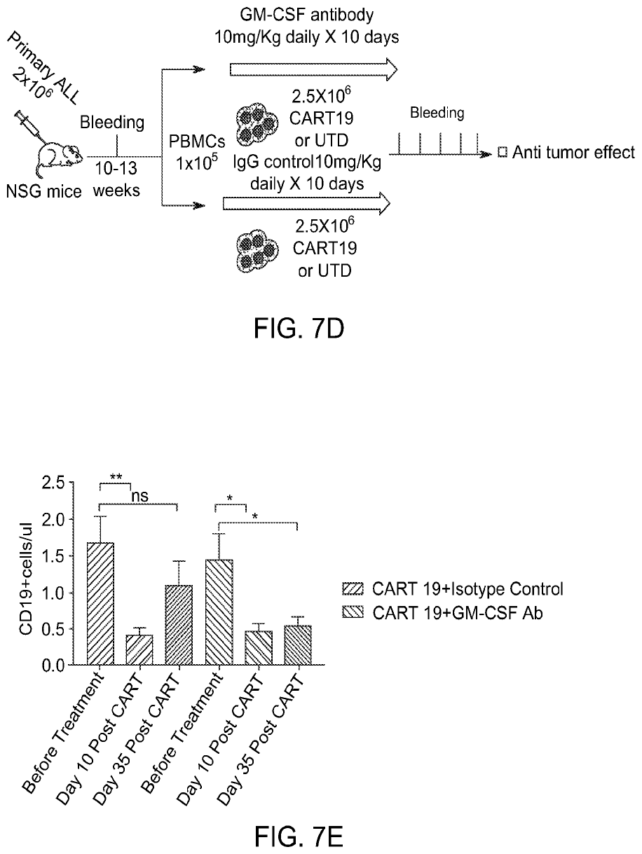

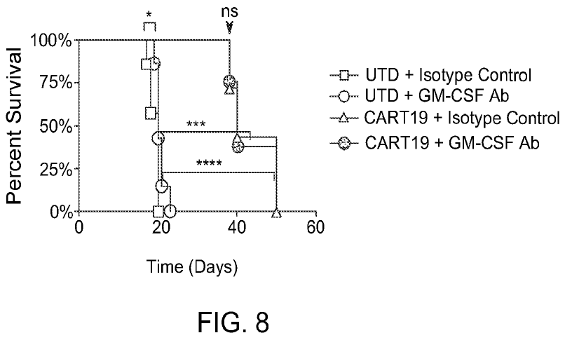

To confirm that GM-CSF depletion does not inhibit CART19 effector functions, we investigated the role of GM-CSF neutralization with lenzilumab on CART19 antitumor activity in xenograft models. First, a relapse model intended to vigorously investigate whether the antitumor activity of CART19 cells was impacted by GM-CSF neutralization was used. NSG mice were injected with 1×10⁶ luciferase+ NALM6 cells and then imaged 6 days later, allowing sufficient time for mice to achieve very high tumor burdens. Mice were randomized to receive a single injection of either CART19 or UTD cells and 10 days of either isotype control antibody or lenzilumab (FIG. 7A). GM-CSF assay on serum collected 8 days after CART19 injection revealed that lenzilumab successfully neutralizes GM-CSF in the context of CART19 therapy (FIG. 7B). Bioluminescence imaging one week after CART19 injection showed that CART19 in combination with lenzilumab effectively controlled leukemia in this high tumor burden relapse model and significantly better than control UTD cells (FIG. 7C). Treatment with CART19 in combination with lenzilumab resulted in potent anti-tumor activity and improved overall survival, similar to CART19 with control antibody despite neutralization of GM-CSF levels, indicating that GM-CSF does not impair CAR-T cell activity in vivo (FIG. 8). Second, these experiments were performed in a primary ALL patient derived xenograft model, in the presence of human PBMCs as this represents a more relevant heterogeneous model. After conditioning chemotherapy with busulfan, mice were injected with blasts derived from patients with relapsed ALL. Mice were monitored for engraftment for several weeks through serial tail vein bleedings and when the CD19+ blasts in the blood were >li.tL, mice were randomized to receive CART19 or UTD treatment in combination with PBMCs with either lenzilumab plus an anti-mouse GM-CSF neutralization antibody or isotype control IgG antibodies starting on the day of CART 19 injection for 10 days (FIG. 7D). In this primary ALL xenograft model, GM-CSF neutralization in combination with CART19 therapy resulted in a significant improvement in leukemic disease control sustained over time for more than 35 days post CART19 administration as compared to CART19 plus isotype control (FIG. 7E). This suggests that GM-CSF neutralization may play a role in reducing relapses and increasing durable complete responses after CART19 cell therapy.

### GM-CSF CRISPR Knockout CAR-T Cells Exhibit Reduced Expression of GM-CSF, Similar Levels of Key Cytokines, and Enhanced Anti-Tumor Activity.

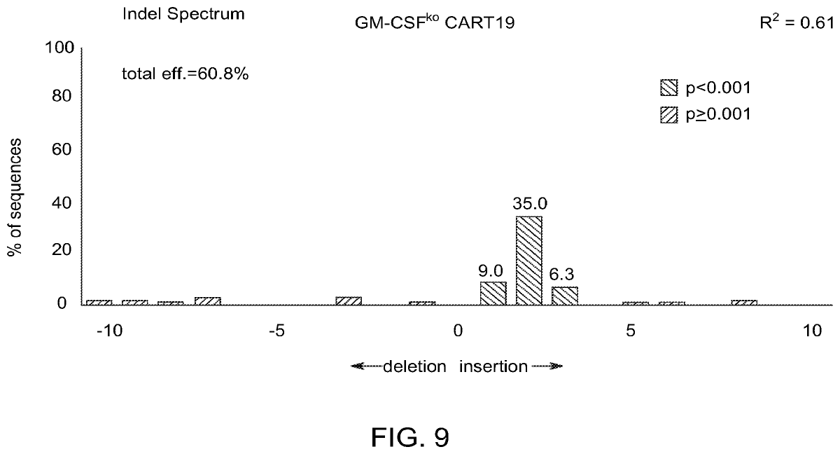

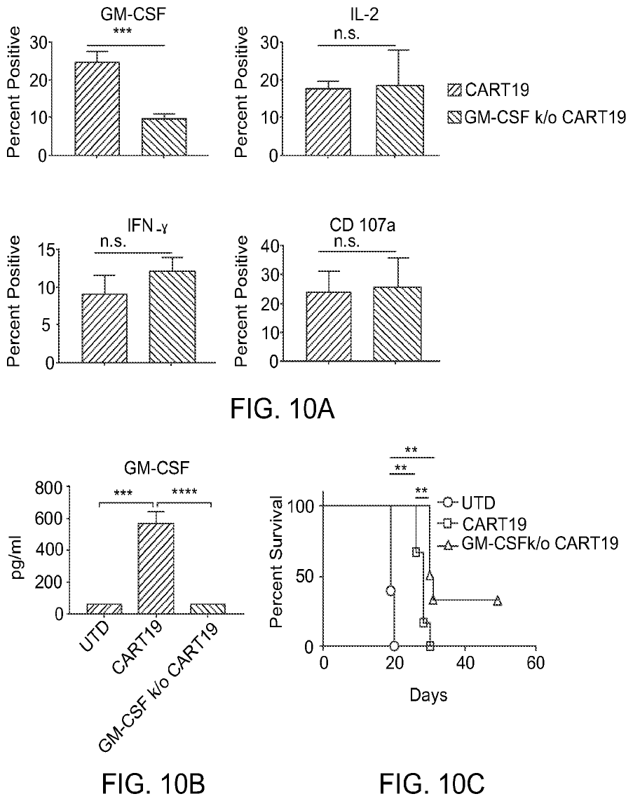

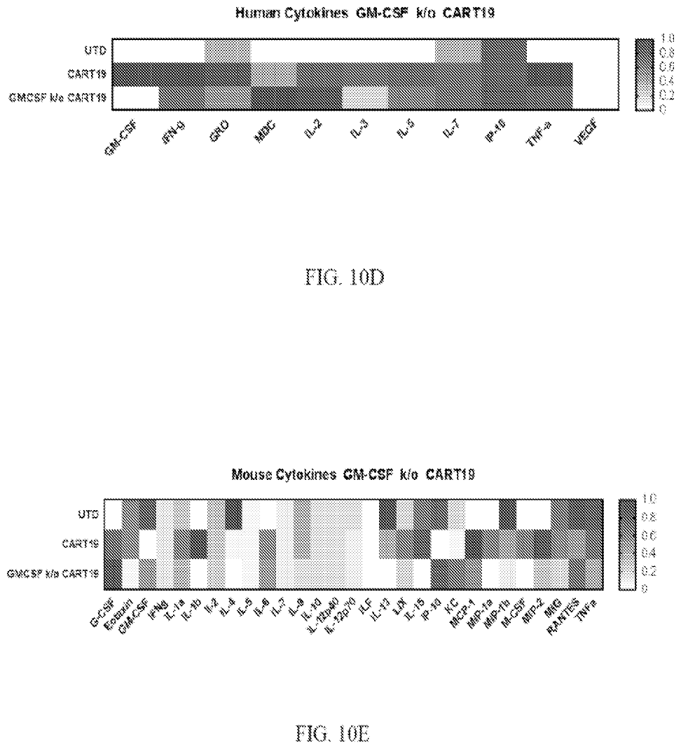

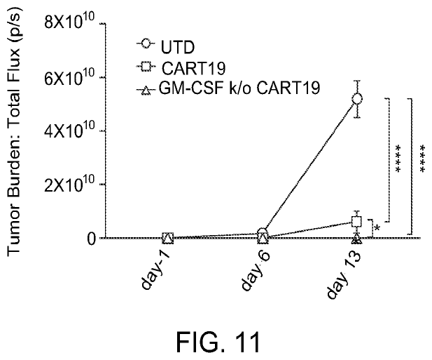

To confidently exclude any role for GM-CSF critical in CAR-T cell function, we disrupted the GM-CSF gene during CAR-T cell manufacturing using a gRNA that has been reported to yield high efficiency, cloned into a CRISPR lentivirus backbone. Using this gRNA, we achieved around 60% knockout efficiency in CART19 cells (FIG. 9). When CAR-T cells were stimulated with the CD19+ cell line NALM6, GM-CSF^{k/o} CAR-T cells produced statistically significantly less GM-CSF compared to CART19 with a wild-type GM-CSF locus (“wild type CART19 cells”). GM-CSF knockout in CAR-T cells did not impair the production of other key T cell cytokines, including IFN-y, IL-2, or CAR-T cell antigen specific degranulation (CD107a) (FIG. 10A) but did exhibit reduced expression of GM-CSF (FIG. 10B). To confirm that GM-CSF^{k/o} CAR-T cells continue to exhibit normal functions, we tested their in vivo efficacy in the high tumor burden relapsing xenograft model of ALL (as described in FIG. 7A). In this xenograft model, utilization of GM-CSF^{k/o} CART19 instead of wild type CART19 markedly reduced serum levels of human GM-CSF at 7 days after CART19 treatment (FIG. 10B). Bioluminescence imaging data implied that GM-CSF^{k/o} CART19 cells show enhanced leukemic control compared to CART19 in this model (FIG. 11). Importantly, GM-CSF^{k/o} CART19 cells demonstrated significant improvement in overall survival compared to wild type CART19 cells (FIG. 10C). Other than GM-CSF, no statistically significantly alterations in either human (FIG. 10D) or mouse (FIG. 10E) cytokines were detected. Together, these results confirm FIGS. 6 and 7, indicating that GM-CSF depletion does not impair cytokines that are critical to CAR-T efficacy functions. In addition, the results in FIG. 10 indicate that GM-CSFk/o CART may represent a therapeutic option for “built in” GM-CSF control as a modification during CAR-T cell manufacturing.

### Patient Derived Xenograft Model for Neurotoxicity and Cytokine Release Syndrome

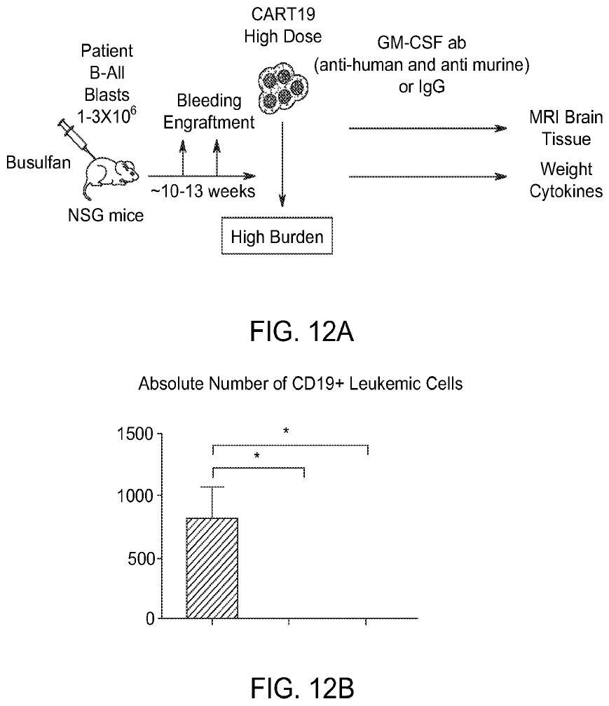

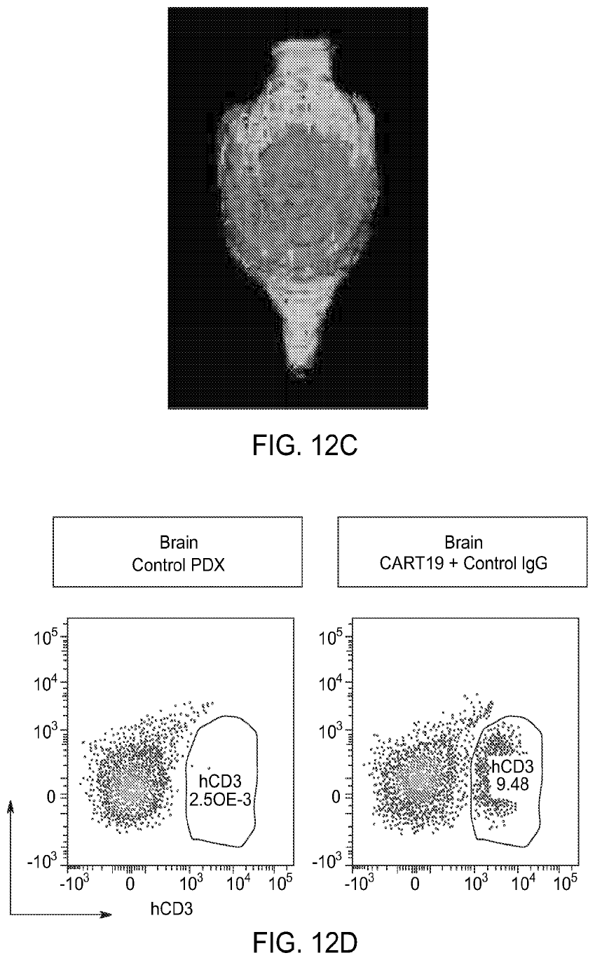

In this model, conditioned NSG mice were engrafted with primary ALL blasts and monitored for engraftment for several weeks until they developed high disease burden (FIG. 12A). When the level of CD19+ blasts in the peripheral blood was ≥10/μL, mice were randomized to receive different treatments as indicated (FIG. 12A). Treatment with CART19 (with control IgG antibodies or with GM-CSF neutralizing antibodies) successfully eradicated the disease (FIG. 12B). Within 4-6 days after treatment with CART19, mice began to develop motor weakness, hunched bodies, and progressive weight loss; symptoms consistent with CRS and NT. This was associated with elevation of key serum cytokines 4-11 days post CART19 injection similar to what is seen in human CRS after CAR-T cell therapy (including human GM-CSF, TNF-a, IFN-y, IL-10, IL-12, IL-13, IL-2, IL-3, IP-10, MDC, MCP-1, MIP-la, MIP-1 , and mouse IL-6, GM-CSF,

IL-4, IL-9, IP-10, MCP-1, and MIG). These mice treated with CART19 also developed NT as indicated by brain MRI analyses revealing abnormal T1 enhancement, suggestive of blood-brain barrier disruption and possibly brain edema (FIG. 12D), together with flow cytometric analysis of the harvested brains revealing infiltration of human CART19 cells (FIG. 12E). In addition, RNA-seq analyses of brain sections harvested from mice that developed these signs of NT showed significant upregulation of genes regulating the T cell receptor, cytokine receptors, T cell immune activation, T cell trafficking, and T cell and myeloid cell differentiation (Table 1).

TABLE 1
Table of canonical pathways altered in brains from patient
derived xenografts after treatment with CART19 cells.

| Canonical Pathway | Adj P-Value | Genes |
|---|---|---|
| regulation of immune | 9.45E−14 | IFITM1, ITGB2, TRAC, ICAM3, CD3G, PTPN22, CD3E, |
| response (G0:0050776) | | ITGAL, SAMHD1, SLA2, CD3D, ITGB7, SLAMF6, B2M, |
| | | NPDC1, CD96, BTN3A1, ITGA4, SH2D1A, HLA-B, HLA-C, |
| | | BTN3A2, HLA-A, CD8B, SELL, CD8A, CD226, CD247, |
| | | CLEC2D, HCST, B1RC3 |
| cytokine-mediated | 1.36E−12 | IFITM1, SP100, TRADD, ITGB2, IL2RG, SAMHD1, IL27RA, |
| signaling pathway | | OASL, CNN2, IL18RAP, RIPK1, CCR5, IL12RB1, B2M, GBP1, |
| (G0:0019221) | | IL6R, JAK3, CCR2, IL32, ANXA1, IL4R, TGFB1, IL10RB, |
| | | IL10RA, STAT2, PRKCD, HLA-B, HLA-C, IL16, HLA-A, |
| | | TNFRSF1B, CD4, IRF3, OAS2, IL2RB, FAS, TNFRSF25, |
| | | LCP1, P4HB, IL7R, MAP3K14, CD44, IL18R1, IRF9, MYD88, |
| | | B1RC3 |
| T cell receptor complex | 1.30E−11 | ZAP70, CD4, CD6, CD8B, CD8A, CD3G, CD247, CD3E, |
| (G0:0042101) | | CD3D, CARD11 |
| T cell activation | 2.07E−11 | ITK, RHOH, CD3G, NLRC3, PTPN22, CD3E, SLA2, CD3D, |
| (G0:0042110) | | CO2, ZAP70, CD4, PTPRC, CD8B, CD8A, LCK, CD28, LCP1, |
| | | LAT |
| regulation of T cell | 2.46E−10 | PTPN22, LAX1, CCDC88B, CD2, CD4, LCK, SIT1, TBX21, |
| activation (G0:0050863) | | TIGIT, JAK3, LAT, PAG1, CCR2 |
| T cell receptor signaling | 4.35E−08 | ITK, BTN3A1, TRAC, WAS, CD3G, PTPN22, BTN3A2, CD3E, |
| pathway (G0:0050852) | | CD3D, ZAP70, CD4, PTPRC, LCK, GRAP2, LCP2, CD247, |
| | | CARD11, LAT, PAG1 |
| positive regulation of | 1.57502E−07 | GBP5, ANXA1, TGFB1, CYBA, PTPN22, PARK7, TMEM173, |
| cytokine production | | CCDC88B, MAVS, CD6, IRF3, CD28, RIPK1, SLAMF6, CD46, |
| (G0:0001819) | | IL12RB1, TIGIT, IL6R, CARD11, MYD88, CCR2 |
| T cell differentiation | 2.36E−07 | ZAP70, CD4, ANXA1, PTPRC, CD8A, LCK, CD28, RHOH, |
| (G0:0030217) | | PTPN22, CD3D |
| cytokine receptor activity | 2.43E−07 | IL4R, IL10RB, IL10RA, IL2RG, CD4, CXCR3, IL2RB, CCR5, |
| (G0:0004896) | | IL12RB1, IL7R, IL6R, CD44, CCR2 |
| type I interferon | 3.27E−07 | IFITM1, SP100, IRF3, OAS2, STAT2, HLA-B, HLA-C, HLA-A, |
| signaling pathway | | SAMHD1, IRF9, MYD88, OASL |
| (G0:0060337) | | |
| response to cytokine | 0.0004679 | SIGIRR, IFITM1, SP100, HCLS1, RIPK1, PTPN7, IKBKE, |
| (G0:0034097) | | IL6R, JAK3, IL18R1, MYD88, AES |
| regulation of innate | 0.001452 | GBP5, GFI1, STAT2, ADAM8, NLRC3, PTPN22, SAMHD1, |
| immune response | | B1RC3 |
| (G0:0045088) | | |
| regulation of tumor | 0.003843 | CD2, MAVS, CYBA, NLRC3, PTPN22, RIPK1, SLAMF1 |
| necrosis factor | | |
| production (G0:0032680) | | |
| T cell receptor binding | 0.0102397 | LCK, CD3G, CD3E |
| (G0:0042608) | | |
| regulation of tumor | 0.0124059 | SHARPIN, TRADD, CASP4, RIPK1, TRAF1, B1RC3 |
| necrosis factor- | | |
| mediated signaling | | |
| pathway (G0:0010803) | | |
| positive regulation of | 0.0376647 | CD4, HCLS1, RIPK1, EVI2B |
| myeloid leukocyte | | |
| differentiation | | |
| (G0:0002763) | | |

GM-CSF neutralization in vivo ameliorates cytokine release syndrome and neurotoxicity after CART19 therapy in a xenograft model.

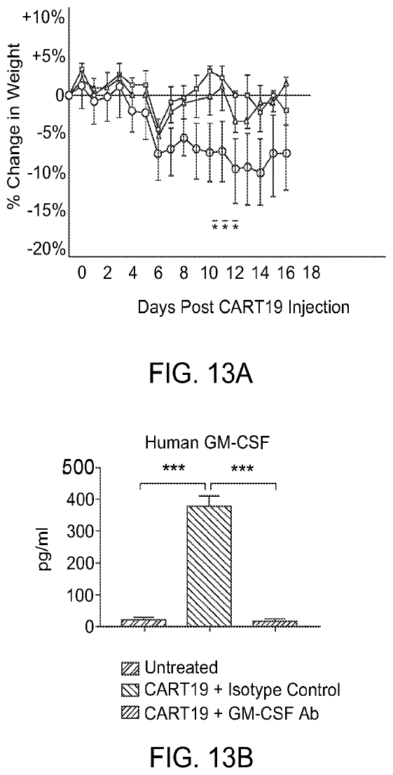

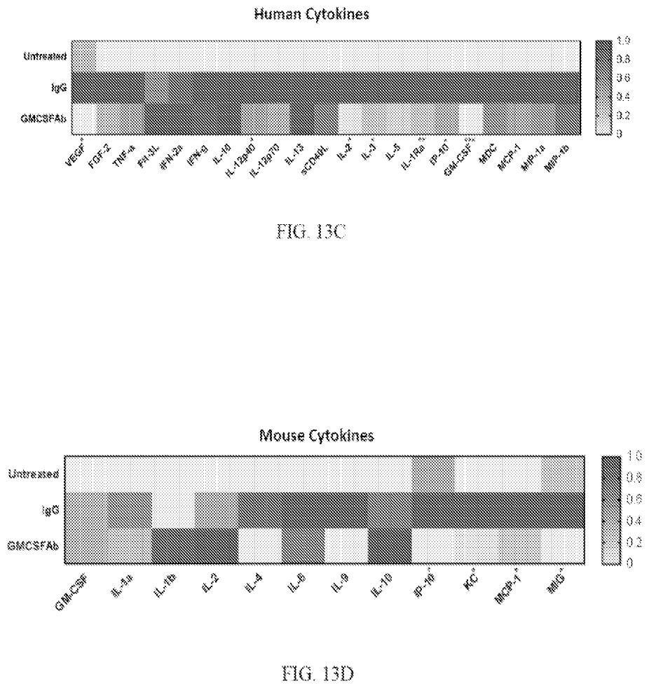

Using the xenograft patient derived model for NT and CRS shown in FIG. 4A, we investigated the effect of GM-CSF neutralization on CART19 toxicities. To rule out the cofounding effect of mouse GM-CSF, mice received CART19 cells in combination with 10 days of GM-CSF antibody therapy (10mg/kg lenzilumab and 10mg/kg anti-mouse GM-CSF neutralizing antibody) or isotype control antibodies. GM-CSF neutralizing antibody therapy prevented CRS induced weight loss after CART19 therapy (FIG. 13A). Cytokine analysis 11 days after CART19 cell therapy showed that human GM-CSF was neutralized by the antibody (FIG. 13B). In addition, GM-CSF neutralization resulted in significant reduction of several human (IP-10, IL-3, IL-2, IL-1Ra, IL-12p40, VEGF, GM-CSF) (FIG. 5C) and mouse (MIG, MCP-1, KC, IP-10) (FIG. 13D) cytokines. Interferon gamma-induced protein (IP-10, CXCL1O) is produced by monocytes among other cell types and serves as a chemoattractant for numerous cell types including monocytes, macrophages, and T cells. IL-3 plays a role in myeloid progenitor differentiation. IL-2 is a key T cell cytokine. Interleukin-1 receptor antagonist (IL-1Ra) inhibits IL-1. (IL-1 is produced by macrophages and is a family of critical inflammatory cytokines.) IL-12p40 is a subunit of IL-12, which is produced by macrophages among other cell types and can encourage Thl differentiation. Vascular endothelial growth factor (VEGF) encourages blood vessel formation. Monokine induced by gamma interferon (MIG, CXCL9) is a T cell chemo attractant. Monocyte chemoattractant protein 1 (MCP-1, CCL2) attracts monocytes, T cells, and dendritic cells. KC (CXCL1) is produced by macrophages among other cell types and attracts myeloid cells such as neutrophils. There was also a trend in reduction of several other human and mouse cytokines after GM-CSF neutralization. This suggests that GM-CSF plays a role in the downstream activity of several cytokines that are instrumental in the cascade that results in CRS and NT.

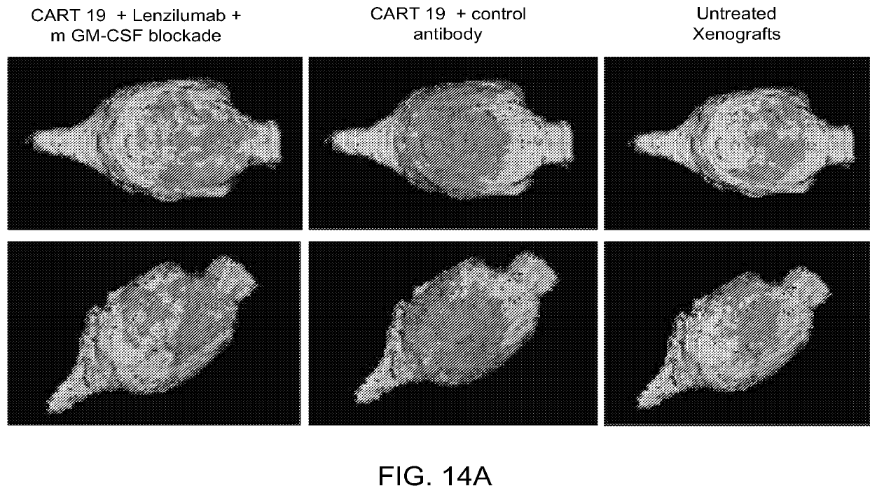

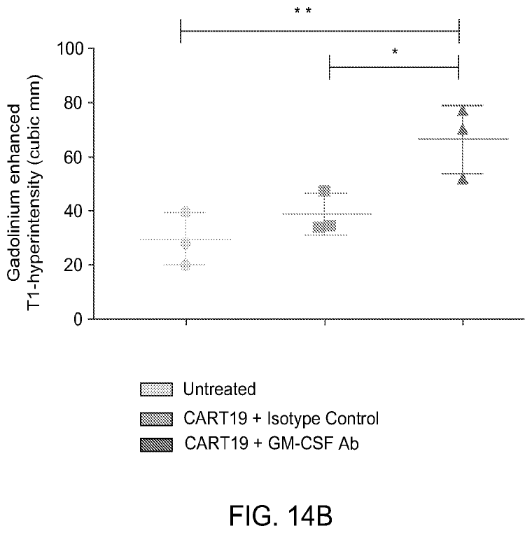

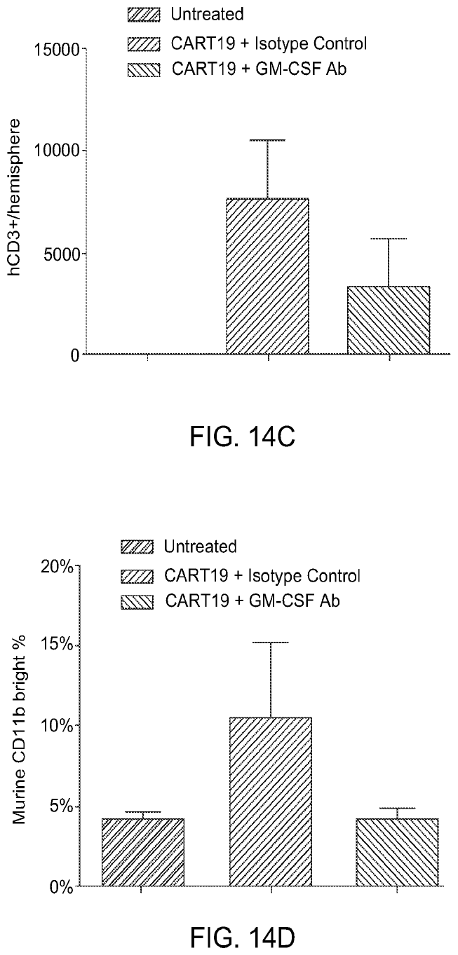

Brain MRIs 5 days after CAR19 treatment showed that GM-CSF neutralization reduced T1 enhancement as a measure of brain inflammation, blood-brain barrier disruption, and possibly edema, compared to CART19 plus control antibodies. The MRI images after GM-CSF neutralization (with lenzilumab and anti-mouse GM-CSF antibody) were similar to baseline pre-treatment scans, suggesting that GM-CSF neutralization effectively helped abrogated the NT associated with CART19 therapy (FIGS. 14A, 14B). Using human ALL blasts and human CART19 in this patient-derived xenograft model, GM-CSF neutralization after CART19 reduced neuro-inflammation by 75% compared to CART19 plus isotype controls (FIG. 14B). This is a significant finding, and the first time it has been demonstrated in vivo that the NT caused by CART19 can be effectively abrogated. Human CD3 T cells were present in the brain after CART19 therapy as assayed by flow cytometry, and with GM-CSF neutralization, there was a trend toward reduction in brain CD3 T cells (FIG. 14C). Finally, a trend in reduction of CD1 lb+bright macrophages was observed in the brains of mice receiving GM-CSF neutralization during CAR-T cell therapy compared to isotype control during CAR-T therapy (FIG. 14D), implicating that GM-CSF neutralization helps reduce macrophages within the brain.

### Example 3-A

### Combination Therapy with GM-CSF Gene KO in CART19 Cells (GM-CSF^{k/o} CART19) and hGM-CSF Neutralizing Antibody (Lenzilumab) for CAR19 T Derived GM-CSF

Lenzilumab (e.g., 10 mg/kg or up to 30 mg/kg or 1,800 mg flat dosing) is administered to a subject in combination with GM-CSF^{k/o} CART19 cells. GM-CSF^{k/o} CAR-T cells help control GM-CSF release upon contact/binding of CAR-T cells with tumor cells. The reduction of GM-CSF secretion at the tumor site results in less activation and trafficking of inflammatory myeloid cells and reduced levels of MCP-1, IL-6, IP10, KC, MIP-la, MIP-lb, MIG, VEGF, IL-1RA, and IL-12p40 in measured systemically. The reduced cytokine levels prevents or reduces the incidence or severity of CRS and NT. The addition of lenzilumab ensures that GM-CSF is neutralized from all sources and helps deplete MDSCs from the tumor microenvironment. Lenzilumab dosing can be repeated at intervals of every two weeks to insure continued depletion of MDSCs. The combination of GM-CSF^{k/o} CAR-T cells with lenzilumab results in improved response rates, improved progression free survival, and improved overall survival in patients treated with the combination therapy vs. control. The combination therapy also results in lower levels (or elimination) of the toxicities associated with CAR-T cell therapy, including CRS and NT.

### Example 3-B

### Combination Therapy with GM-CSF Gene KO in CART19 Cells (GM-CSF^{k/o} CART19) and hGM-CSF Neutralizing Antibody (Lenzilumab) for CAR19 T Derived GM-CSF

Lenzilumab (/600-1800mg) is administered to a subject in combination with GM-CSF^{k/o} CART19 cells.

Non-Hodgkins Lymphoma cancer patients are pre-conditioned prior to therapy. They are dosed I.V. with anti-hGM-CSF antibody (600-1800mg) followed by 2×10⁶ transduced autologus CD19CART cells (GM-CSF^{KO}). At specific times after treatment effects are assessed e.g., safety, blood chemistry, neurologic assessments, disease status. The treatment may be repeated on a monthly basis until there is no further detectable cancer or there is a significant reduction in cancer load.

### Example 4

### Recombinant Anti-hGM-CSF Antibody, Lenzilumab, Reduces Myeloid Cell Infiltration in the CNS

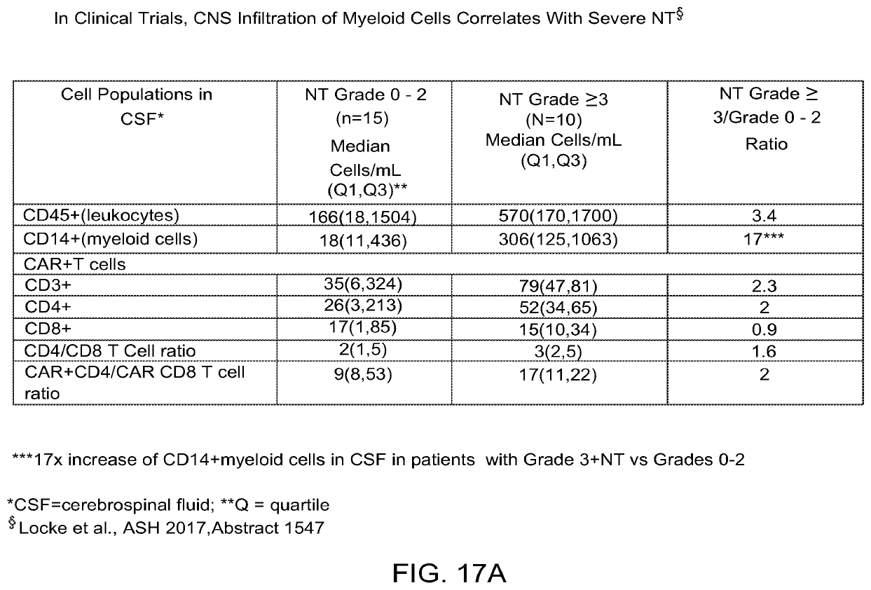

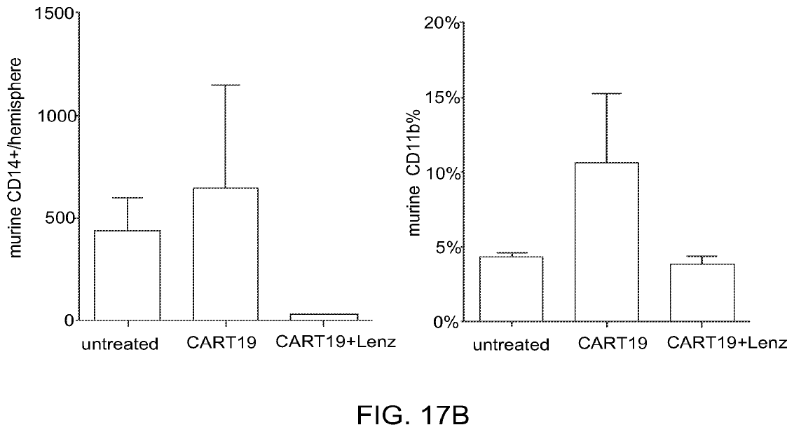

CD14+ cells comprise a greater proportion of the CNS cell population in human patients with grade 3 or above neurotoxicity, as shown in FIG. 17A. Administration of recombinant anti-hGM-CSF antibody (lenzilumab) that binds to and neutralizes human GM-CSF to mice treated with CART19 therapy demonstrated a reduction in CNS infiltration by CD14+ cells and by CD1 1b+ cells, as shown in FIG. 17B in comparison to untreated mice and mice treated only with CART19 therapy. A primary ALL mouse model was used, as detailed below for the NT experiments.

### Primary Patient Derived ALL Xenografts for CRS/NT

Similar to the experiments above, mice were IP injected with 30 mg/kg busulfan. The following day, they received 1-2×10⁶ primary blasts derived from the peripheral blood of patients with relapsed refractory ALL. Mice were monitored for engraftment for 4-6 weeks and when CD19+ cell level was high (>10 cells/0), they received CART19 (2-5×10⁶ cells) and commenced antibody therapy for a total of 10 days, as indicated in the details of the specific experiment. (as described in Example 2). Mice were weighed on daily basis as a measure of their well-being. Brian MRI of the mice was performed 5-6 days post CART injection and tail vein bleeding was performed 4-11 days post CART injection. Brain MRI images were analyzed using Azalyze.

### Example 5

### Gene Editing Technologies to Knockout GM-CSF Genes in T Cells

Several strategies are being pursued by various groups to incorporate gene editing into the development of next-generation chimeric antigen receptor (CAR) T cells for the treatment of various cancers. Severe toxicity (cytokine release syndrome and neurotoxicity) is associated with CAR T cell therapy and can result in poor patient outcomes. A key initiator in the toxicity process seems to be CART cell derived GM-CSF.

Gene-editing (with e.g., engineered nucleases) may be used to KO GM-CSF genes in T cells and/or gene/s encoding proteins essential for GM-CSF gene expression. Nucleases useful for such genome editing include, without limitation, CRISPR-associated (Cas) nucleases, zinc-finger nucleases (ZFNs), transcription activator-like effector (TALE) nucleases, and homing endonucleases (HEs) also known as meganucleases

### Zinc-Finger Nuclease Use for GM-CSF

A GM-CSF gene in CART cells can be inactivated using Zinc Finger Nuclease (ZFN) technology. DNA sequence specific nucleases cleave the GM-CSF gene/s and DNA double strand break repair results in inactivation of the gene/s. The sequence specific nucleases are created by combining sequence specific DNA binding domains (Zinc fingers) with a Fokl endonuclease domain. The targeted nuclease acts as a dimer and two different DNA recognition domains are employed to provide site specific cleavage. Engineering of the Fok 1 endonuclease ensures that heterodimers form rather than homodimers. Thus, the obligate heterodimer Fokl-EL variant provides a higher level of specificity.

Clinical experience to date with gene KO approaches using ZFN technology is limited. However, in a small safety study in which the CCRS receptor was knocked-out using ZFN technology and the T-cells re-introduced into HIV patients, there was a notable survival advantage of the modified T cells vs unmodified when anti-retroviral drug therapy was stopped.

The best effect was observed when biallelic gene disruption was achieved. This suggests that the KO technology that achieves greatest % gene disruption is likely to be the most effective (Singh 2017, Tebas 2014). In some human cell types biallelic targeting efficiency is increased by RAD51 over expression and valproic acid treatment (Takayama 2017).

Exons 1-4 of the human GM-CSF gene can be targeted with ZFNs that form pairs within the chosen target region. A potential advantage to targeting close to the translational initiation codon within the DNA sequence is that it ensures that the gene knockout does not result in a large fragment of protein that is still synthesized. Such protein fragments could have unwanted biological activities.

A variety of tools are available for the identification of potential zinc finger nuclease (ZFN) sites in specific target sequences. An example of such tools can be found at: http://bindr.gdcb.iastate.edu/ZiFiT/. Vectors for the expression of pairs of ZFNs identified in this way (for use in GM-CSF gene KO) are tested in human cells expressing GM-CSF and the effectiveness of gene disruption for each pair is measured by changes in GM-CSF production within a pool of cells. Pairs of ZFNs demonstrating the highest reduction in GM-CSF levels are chosen for testing in human CART cells.

For example, autologous T-cells can be transduced ex vivo with a replication deficient recombinant Ad5 viral vector encoding pairs of the GM-CSF specific ZFNs, resulting in modification of the GM-CSF gene. The vector supports only transient expression of genes encoded by the vector. The two ZFNs bind to a composite bp sequence found specifically in the region chosen for mutagenesis (within exons 1,2, 3 or 4) of the GM-CSF gene . Expression of the GM-CSF-specific ZFNs induces a double stranded break in the cellular DNA which is repaired by cellular machinery leading to random sequence insertions or deletions in the transduced cells. These insertions and deletions disrupt the GM-CSF coding sequence leading to frameshift mutation and termination of protein expression.

### The T Cell Manufacture/Patient—Specific Sample

Study subjects undergo a 10 liter leukapheresis to collect >10⁹ white blood cells. The leukapheresis product is enriched for CD4+ cells by depleting monocytes via counterflow centrifugal elutriation, and by magnetically depleting CD8+ T-cells, both employing a single-use closed-system disposable set. The resulting enriched CD4+ T-cells are activated with anti-CD3/anti-CD28 mAb coated paramagnetic beads and transduced with vector encoding CAR T and vector encoding ZFNs. Cells are then expanded and cultured in a closed system. T-cell expansion continues after transfer to a WAVE Bioreactor for additional expansion under perfusion conditions. At the end of the culture period, cells are depleted of magnetic beads, washed, concentrated, and cryopreserved.

Primary T cells may also be treated with treated with other agents, e.g., valproic acid in order to increase bi-allelic targeting efficiency of the ZFNs.

Putative Targeting Sequences
Exon 1
ATG TGG CTG CAG AGC CTG CTG CTC TCG GGC (SEQ ID NO: 14)
TAC ACC GAC GTC TCG GAC GAC GAG AGC CCG (SEQ ID NO: 15)
CTC GCC CAG CCC CAG CAC GCA GCC (SEQ ID NO: 14 continued)
GAG CGG GTC GGG GTC GTG CGT CGG (SEQ ID NO: 15 continued)
Exon 2
AAT GAA ACA GTA GAA GTC ATC TCA GAA ATG (SEQ ID NO: 16)
TTA CTT TGT CAT CTT CAG TAG AGT CTT TAC (SEQ ID NO: 17)
GAA GTC ATC TCA GAA ATG TTT GAC (SEQ ID NO: 16 continued)
CTT CAG TAG AGT CTT TAC AAA CTG (SEQ ID NO: 17 continued)
Design Exon 3
GAG CCG ACC TGC CTA CAG ACC CGC CTG GAG (SEQ ID NO: 18)
CTC GGC TGG ACG GAT GTC TGG GCG GAC CTC (SEQ ID NO: 19)
GCC TAC AGA CCCGCCT GGA GCT GTA (SEQ ID NO: 18 continued)
CGG ATG TCT GGGCGGA CCT CGA CAT (SEQ ID NO: 19 continued)
Exon 4
GAA ACT TCC TGT GCA ACC CAG ATT ATC ACC (SEQ ID NO: 20)
CTT TGA AGG ACA CGT TGG GTC TAA TAG TGG (SEQ ID NO: 21)
TGC AAC CCA GAT TATC ACC TTT GAA (SEQ ID NO: 20 continued)
ACG TTG GGT CTA ATAG TGG AAA CTT (SEQ ID NO: 21 continued)

### TALENS

GM-CSF gene/s in T cells can also be inactivated using activator-like effector nucleases (TALENS). TALENS are similar to ZFNs in that they comprise a Fokl nuclease domain fused to a sequence specific DNA-binding domain. The targeted nuclease then makes a double-strand break in the DNA and error-prone repair creates a mutated target gene. TALENS can be easily designed using a simple protein-DNA code that uses DNA binding TALE (transcriptional-activator —like effectors) repeat domains to individual bases in a binding site. The robustness of TALEN means that genome editing is a reliable and facile process (Reyon D., et al., 2012 Nat Biotechnol. 2012 May;30(5):460-5. doi: 10.1038/nbt.2170, which is incorporated herein by reference its entirety.)

By way of examples, some TALE target sequences within Exon 1 of human GM-CSF gene are:

1.
(SEQ ID NO: 22)
TGGCTGCAGAGCCTGCTGCTCTTGGGCACTGTGGCCTGCAGCATC
TCTGCA
2.
(SEQ ID NO: 23)
TTGGGCACTGTGGCCTGCAGCATCTCTGCACCCGCCCGCTCGCCC
AGCCCCA
Examples of TALE target sequences in Exon 4
of human GM-CSF gene:
1.
(SEQ ID NO: 24)
TGTGCAACCCAGATTATCACCTTTGAAAGTTTCAAAGAGAACCTG
AAGGA
2.
(SEQ ID NO: 25)
TCCTGTGCAACCCAGATTATCACCTTTGAAAGTTTCAAAGAGAAC
CTGAA
3.
(SEQ ID NO: 26)
TTATCACCTTTGAAAGTTTCAAAGAGAACCTGAAGGACTTTCTGC
TTGTCA

### CRISPR Cas-9 Mediated GM-CSF Gene KO in Primary T-cells.

The CRISPR (clustered regularly interspaced short palindromic repeats), Cas-9 system is composed of Cas9, a RNA-guided nuclease and a short guide RNA (gRNA) that facilitates the generation of site-specific DNA breaks, which are repaired by cell-endogenous mechanisms. Cas9/gRNA RNP delivery to primary human T-cells results in highly efficient target gene modification. CRISPR/Cas9 mediated methods to knockout the GM-CSF gene are described by Detailed protocols see Oh, S. A., Seki, A., & Rutz, S. (2018) Current Protocols in Immunology, 124, e69. doi: 10.1002/cpim.69, and Seki and Rutz, J Exp. Med. 2018 Vol. 215 No. 3 985-997, each of which is incorporated herein by reference its entirety.

GM-CSF inactivation by gene KO has been reported to reduce cytokine release syndrome and neurotoxicity and improve anti-tumor activity in CAR T treated mice with tumor xenografts (as described by Sterner RM et al., 2018 Blood 2018:blood-2018-10-881722; doi: https://doi.org/10.1182/blood-2018-10-881722), which is incorporated herein by reference its entirety.

### Inactivation of GM-CSF Gene by CRISPR Approach Targeting Exon 1 or 2 or 3 or 4.

Multiple, e.g., 3 Cas9 constructs targeting 3 different sequences within the GM-CSF gene may be used so as to ensure efficient gene inactivation in all samples. (This is easily done with CRISPR compared to other gene editing methods.)

High frequency of bi-allelic KO reported using Cas9 (as described by Zhang, Y., et al. Methods. 2014 September; 69(2): 171-178. doi:10.1016/j.ymeth.2014.05.003, which is incorporated herein by reference its entirety. This high frequency of bi-allelic KO provides a possible advantage.

Other Gene Silencing Technologies for GM-CSF KO in CAR T cells

Other methods that can be used for gene silencing are well known to those ordinary skilled in the art and may include, without limitation, homing endonucleases (HEs) also known as meganucleases, RNA interference (RNAi), short interfering RNS (siRNA), DNA-directed RNA interference (ddRNAi).

Combination of GM-CSF Gene KO in CAR T Cells and Neutralizing Antibody for non-CAR T Derived GM-CSF.

Removal/neutralization of all GM-CSF in patients requires anti-GM-CSF antibody, anti-receptor antibody, or soluble receptor-Fc fusion used in combination with GM-CSF gene KO in CART cells. The CART cells administered, include but are not limited to, GM-CSF^{k/o} CART cells. In one embodiment, the administered GM-CSF^{k/o} CART cells are GM-CSF^{k/o} CART19.

An anti-GM-CSF neutralizing antibody is administered in this combination therapy, including but not limited to Lenzilumab. Lenzilumab is a novel, high affinity, recombinant human, neutralizing anti-hGM-CSF antibody. Studies in non-human primates have shown that this antibody is safe when repeat-dosed, even at doses as high as >100 mg/kg/wk. for 6 weeks. This antibody is also safe in humans when repeat-dosed (7 doses of 400 mg/dose, over 24 weeks to severe asthmatics). This antibody can be used in combination with GM-CSF KO CART cell therapy in cancer patients providing complete neutralization of human GM-CSF. Cancer patients are dosed I.V. with anti-hGM-CSF antibody (600-1800mg) followed by 2×10⁶ CART cells (GM-CSF KO). At specific times after treatment effects are assessed, e.g., safety, blood chemistry, neurologic assessments, disease status. The treatment may be repeated on a monthly or 3 monthly basis and may result in disease remission and improved progression free survival.

GM-CSF can also be neutralized using an anti-human GM-CSF receptor alpha (Ra) antibody (as described in Minter, RR, et al. 2012 DOI:10.1111/j.1476-5381.2012.02173.x). Cancer patients are dosed I.V. with anti-hGM-CSF receptor antibody (70-700 mg) followed by 2×10⁶ CAR T cells (GM-CSF^{KO}). At specific times after treatment effects are assessed, e.g., safety, blood chemistry, neurologic assessments, disease status. Treatment results in disease remission and improved progression free survival. The treatment may be repeated on a monthly basis until there is no further detectable cancer or there is a significant reduction in cancer load.

### Example 6

### GM-CSF Disruption in CART Cells Ameliorates CART Cell Activation and Reduces Activation-induced Cell Death

It was hypothesized that GM-CSF depletion in CART cells results in reduced activation-induced cell death (AICD) and enhanced anti-tumor activity independent of the effect on myeloid cell activation. In the present Example, how GM-CSF disruption in CART cells impacts their functions was tested.

### Methods

Cell lines. The acute lymphoblastic leukemia cell line NALM6 was purchased from ATCC (CRL-3273, Manassas, VA, USA). Cell lines were cultured in R10 (RPMI 1640, Gibco, Gaithersburg, MD, US), 10% Fetal Bovine Serum (FBS, Millipore Sigma, Ontario, Canada), and 1% Penicillin-Streptomycin-Glutamine (Gibco, Gaithersburg, MD, US). Cell lines are kept in culture after 20 passages, and fresh aliquots are thawed every 7-8 weeks. The use of recombinant DNA in the laboratory was approved by the Mayo Clinic Institutional Biosafety Committee (IBC).

CART Cells. Generation of constructs, lentiviral production, titration, GM-CSF^{k/o} CART19 cells, and T cell functional experiments were performed as previously described in Sterner RM, Sakemura R, Cox MJ, et al. GM-CSF inhibition reduces cytokine release syndrome and neuroinflammation but enhances CAR-T cell function in xenografts. Blood. 2019;133(7):697-709 and Sterner RM, Cox MJ, Sakemura R, Kenderian SS. Using CRISPR/Cas9 to Knock Out GM-CSF in CAR-T Cells. J Vis Exp. 2019(149), each of which is incorporated herein by reference in its entirety.

Flow Cytometric Analysis. Extracellular staining, acquisition, and gating were previously described in Sterner RM, et al. Blood. 2019;133(7):697-709, which is incorporated herein by reference in its entirety. The following antibodies were used: anti-CD116 (GM-CSFRa) (clone 4H1) FITC (BioLegend, San Diego, CA, USA), anti-CD131 (GM-CSFR(3) (clone 1C1) PE (BioLegend, San Diego, CA, USA), CD262 (clone DJR2-4) PE (BioLegend, San Diego, CA, USA), CD3 (clone OKT3) BV421 (BioLegend, San Diego, CA, USA), CD3 (clone SK7) APC-H7 (BD Pharmingen, San Jose, CA, USA), CD4 (clone OKT4) FITC (eBioscience, San Diego, CA, USA), CD8 (clone SK1) PerCP (BioLegend, San Diego, CA, USA), CD3 BV650 (BioLegend, San Diego, CA, USA), CD45 BV421 (BioLegend, San Diego, CA, USA), CD20 PE (BioLegend, San Diego, CA, USA), CD25 PE-Cy7 (BioLegend, San Diego, CA, USA), CD69 BV785 (BioLegend, San Diego, CA, USA), H1A-DR APC-Fire/750 (BioLegend, San Diego, CA, USA), and mCD45 PE (BioLegend, San Diego, CA, USA). Absolute quantification was obtained using volumetric measurement.

Sequencing. RNA isolation and analysis were previously described in Sterner RM, et al. Blood. 2019;133(7):697-709, which is incorporated herein by reference in its entirety. DNA was isolated using PureLink Genomic DNA Mini Kit (Invitrogen, Carlsbad, CA, USA), prepared with Agilent SureSelectXT (Santa Clara, CA, USA), and sequenced on Illumina HiSeq 4000 (Illumina, San Diego, CA, USA) by the Medical Genome Facility Genome Analysis Core (Mayo Clinic, Rochester, MN, USA). Burrows-Wheeler Aligner, as described in Li H, Durbin R. Fast and accurate long-read alignment with Burrows-Wheeler transform. Bioinformatics. 2010;26(5):589-595, which is incorporated herein by reference in its entirety, and Genome Analysis Toolkit, as described in McKenna A, Hanna M, Banks E, et al. The Genome Analysis Toolkit: a MapReduce framework for analyzing next-generation DNA sequencing data. Genome Res. 2010;20(9):1297-1303, which is incorporated herein by reference in its entirety, were used to align reads to GRCh28 and call variants. SAS 9.4 (SAS Institute Inc., Cary, NC, USA) was used to find differences and filter by genomic prevalence (allele frequency ≤1%), as described in Genomes Project C, Auton A, Brooks LD, et al. A global reference for human genetic variation. Nature. 2015;526(7571):68-74, which is incorporated herein by reference in its entirety. CRISPR/Cas9 target online predictor (CCTop) off-target predictions were cross-referenced, as described in Stemmer M, et al. CCTop: An Intuitive, Flexible and Reliable CRISPR/Cas9 Target Prediction Tool. PLoS One. 2015;10(4):e0124633, which is incorporated herein by reference in its entirety. Single nucleotide variants (SNVs) or insertions/deletions (indels) were compared between knockouts and controls. Statistical testing using Wilcoxon signed-rank test performed using GraphPad Prism version 8.1.1 for Windows (GraphPad Software, La Jolla, CA, USA, www.graphpad.com), as described in Iyer V, Boroviak K, Thomas M, et al. No unexpected CRISPR-Cas9 off-target activity revealed by trio sequencing of gene-edited mice. PLoS Genet. 2018;14(7):e1007503, which is incorporated herein by reference in its entirety.

Data Sharing Statement. Sequencing data are available at BioProject PRJNA623000.

Flow Cyometric Analysis. All anti-human and anti-mouse antibodies were purchased from BioLegend, eBioscience or BD Biosciences (San Diego, CA, USA). Cells were always washed twice in phosphate-buffered saline supplemented with 2% FBS (Millipore Sigma, Ontario, Canada) and 1% sodium azide (Ricca Chemical, Arlington, TX, USA). In all analyses, populations of interest were gated based on forward vs. side scatter characteristics, followed by singlet gating, and live cells were gated following staining with LIVE/DEAD™ Fixable Aqua Dead Cell Stain Kit (Invitrogen, Carlsbad, CA, USA). Surface expression of CAR was detected by staining with a goat anti-mouse F(ab′) 2 antibody (Invitrogen, Carlsbad, CA, USA). Expression of GM-CSF receptors α and β was detected with anti-human CD116 (4H1) FITC (305906, BioLegend, San Diego, CA) and anti-human CD131 (1C1) PE (306104, BioLegend, San Diego, CA), respectively. Cytometric data were acquired using a CytoFLEX Flow Cytometer (Beckman Coulter, Chaska, MN, USA). Gating was performed using Kaluza version 2.1 (Beckman Coulter, Chaska, MN, USA).

TUNEL assay. One million CART19 cells or GM-CSF^{k/o} CART19 at 1×10⁶/mL were co-cultured with 1 million irradiated NALM6 at 0.5×10⁶/mL for 0, 1, 2, and 4 hours. To assess these samples with the TUNEL assay, the manufacturer's protocol was followed (APO-BRDU MilliporeSigma, St. Louis, MO, USA). Briefly, the cells were fixed at each timepoint and stored at −20° C. To measure apoptosis, the cells were then washed and incubated with DNA labeling solution containing TdT and Br-dUTP. Then the cells were labeled with anti-BrDU-FITC and propridium iodide. The samples were then run on the CytoFLEX flow cytometer with positive and negative controls provided in the kit (Beckman Coulter, Chaska, MN, USA).

Apoptosis assays. CART19 or GM-CSF^{k/o} CART19 cells were stimulated with PMA/ionomycin, CD19+ cell line NALM6 or CD3/CD28 beads at different time points (0 hr, 1 hr, 2 hr, 4 hr, 6 hr) on a 1:1 ratio. Then, cells were spin and washed with flow buffer, followed by incubation in the dark with the following antibodies: CD3 (SK7) APC-Cy7 (560176, BioLegend, San Diego, CA), Annexin V PE (556421, BD Biosciences, San Jose, CA), 7-AAD (559925, BD Biosciences, San Jose, CA). Then, the expression of Annexin V and 7-AAD was measured via flow cytometry. Three biological replicates of unstimulated and stimulated CART19 and GM- CSF^{ko} CART19 cells were included. For assays including blocking antibodies, TRAIL-R2 (DR5) Fc (10140-T2-100, R&D Systems, Minneapolis, MN) and Fas Fc (326-FS-050, R&D Systems, Minneapolis, MN) were added at a dose of 10 ng/mL.

Western blot. For immunoblot assays, irradiated target cell line NALM6 was co-cultured at a 1:1 ratio with CART19 or GM-CSF^{ko} CART19 cells at different time points (0 hr, 2 hr, 4 hr, 6 hr). Cell pellets were washed with PBS and lysed in 100 uL of RIPA buffer (89900, Thermo Fisher, Waltham, MA, USA), and protein concentration was measured by BCA protein assay (23255, Thermo Fisher, Waltham, MA, USA). SDS-PAGE gels were used to resolve 30 ug cell lysates, and proteins were transferred to Nitrocellulose membranes via wet transfer. Nitrocellulose membranes were blocked with 5% BSA in TBST for 1 hr at room temperature. Membranes were incubated overnight at 4° C. with the following antibodies: Rabbit BID (2002, Cell Signaling, Danvers, MA, USA) (dilution 1:1000) and Rabbit (3-Actin D6A8 (8457, Cell Signaling, Danvers, MA, USA). Membranes were washed with TBST and incubated with HRP-conjugated secondary antibodies at a dilution of 1:1000 for 1 hr at room temperature. Blots were revealed using the SuperSignal West Pico Plus Chemiluminescent substrate (34579,Thermo Fisher, Waltham, MA, USA).

Animal Models. 6-8 week old non-obese diabetic/severe combined immunodeficient mice bearing a targeted mutation in the interleukin (IL)-2 receptor gamma chain gene (NSG) mice were purchased from Jackson Laboratories (Jackson Laboratories, Bar Harbor, ME, USA) and then maintained at the Mayo Clinic animal facility. All animal experiments were performed under an IACUC approved protocol (A00001767). Mice were maintained in an animal barrier space that is approved by the IBC for BSL2+ level experiments (IBC #HIP00000252.20). Mice were intravenously injected with 1.0×10⁶ luciferase+ JeKo-1 cells. Fourteen days after injection, mice were imaged with a bioluminescent imager using an IVIS® Lumina S5 Imaging System (PerkinElmer, Hopkinton, MA, USA) to confirm engraftment. Imaging was performed 10 minutes after the intraperitoneal injection of 10 μL/g D-luciferin (15 mg/mL, Gold Biotechnology, St. Louis, MO, USA). Mice were then randomized based on their bioluminescence imaging to receive different treatments as outlined in the separate specific experiments. Mice were euthanized for necropsy when moribund.

### Results

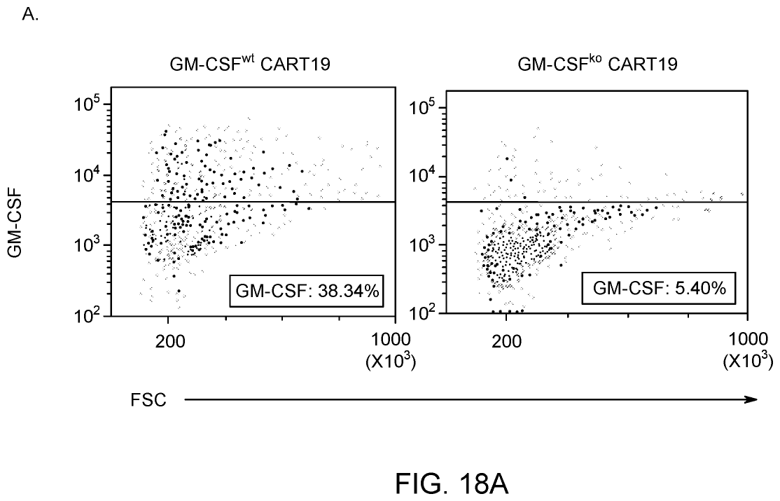

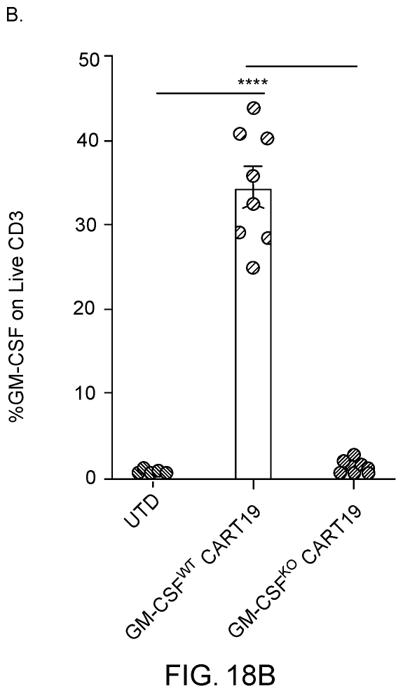

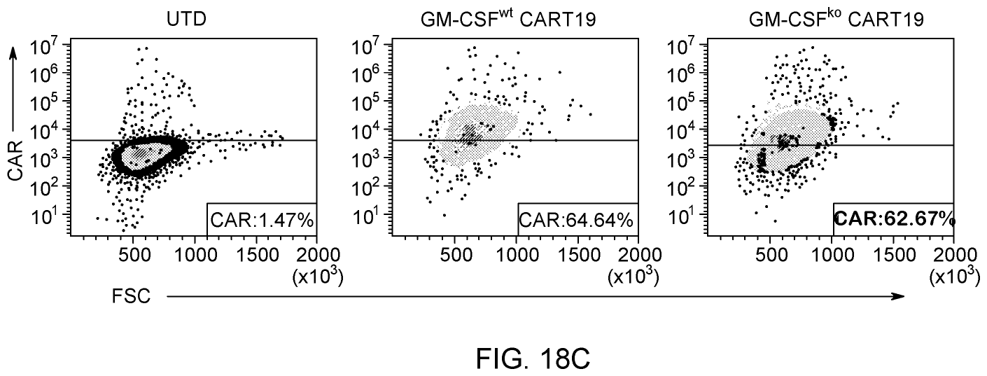

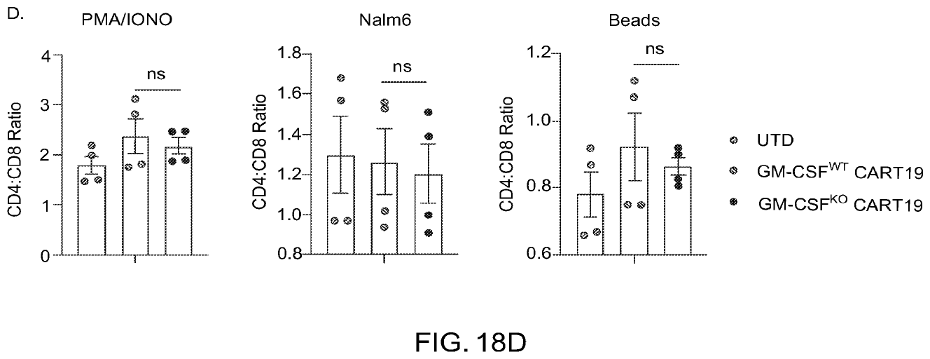

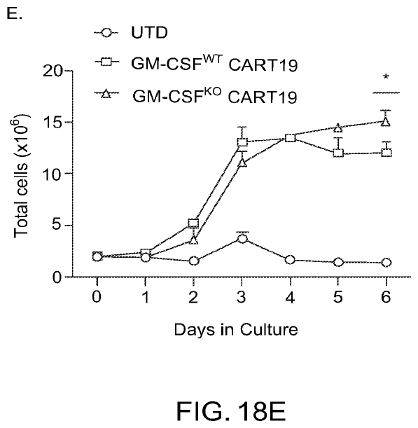

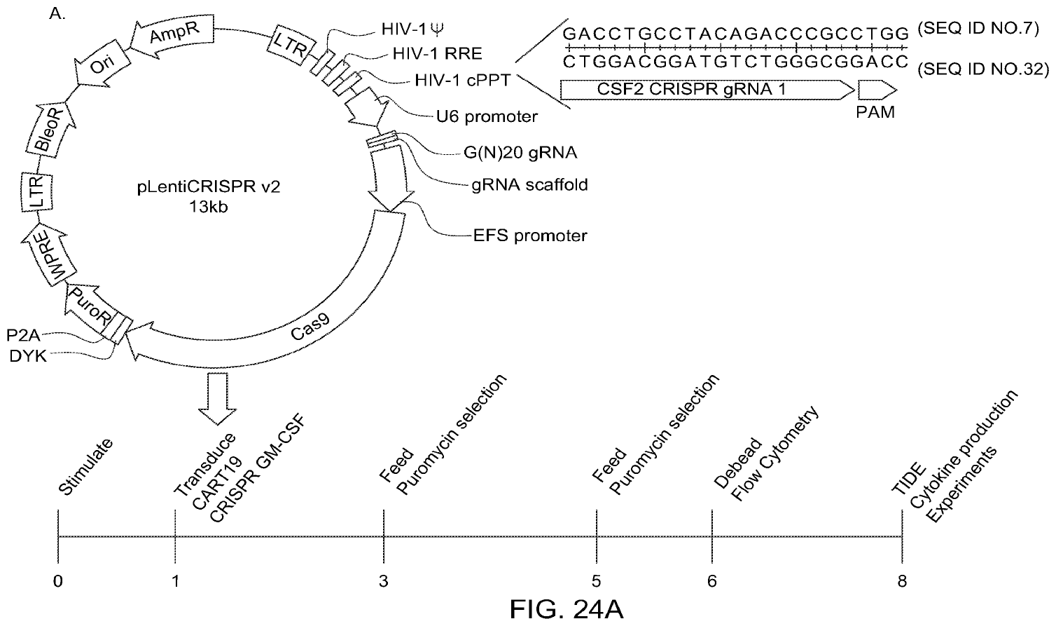

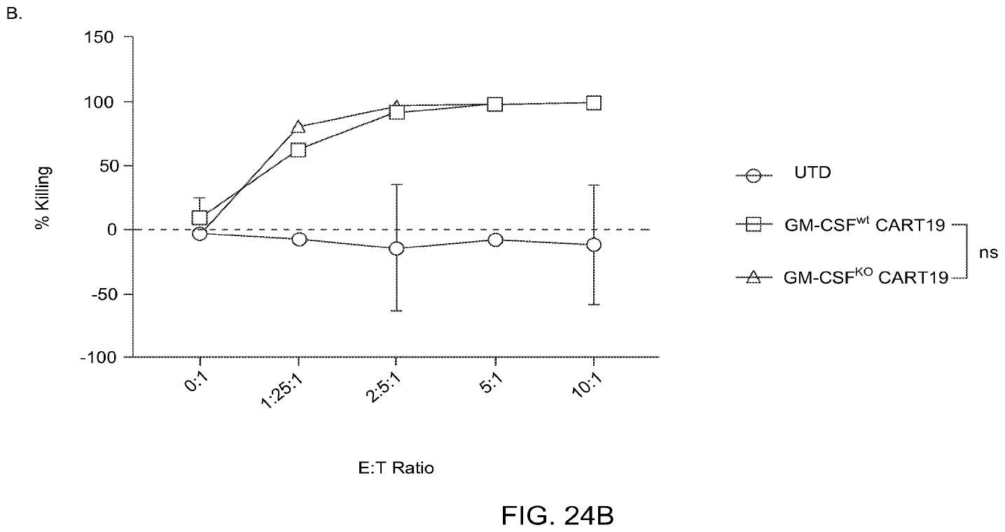

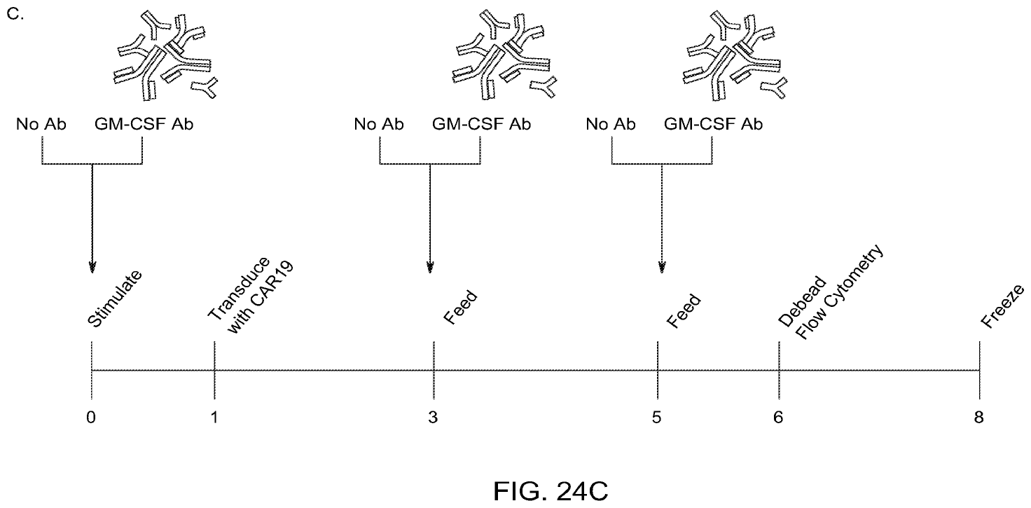

Knock out of GM-CSF in CART19 cells enhances their antigen specific proliferation. In order to generate GM-CSF^{k/o} CART cells, CRISPR/Cas9 was used to disrupt GM-CSF (CSF2) in CART19 cells, and generated CART cells that produced little to no GM-CSF upon activation through their CAR (FIGS. 9 and 18A-18B, see method section, CART Cells, FIG. 24A). GM-CSF disruption in CART cells did not affect the transduction efficiency of T cells (FIG. 18C), change the composition of CART cell product (CD4:CD8 ratio) at rest or upon activation (FIG. 18D), or alter CART cell antigen specific killing (FIG. 24B). However, GM-CSF^{k/o} CART19 cells exhibited superior antigen specific proliferation compared to GM-CSF^{wt} CART19 when stimulated through the CAR19 via a co-culture with the irradiated CD19+ NALM6 cells (FIG. 18D). Importantly, while antigen specific proliferation of GM-CSF^{k/o} CART19 and GM-CSF^{wt} CART19 was initially similar, it significantly improved after 5 days following the initial stimulation (FIG. 18E).

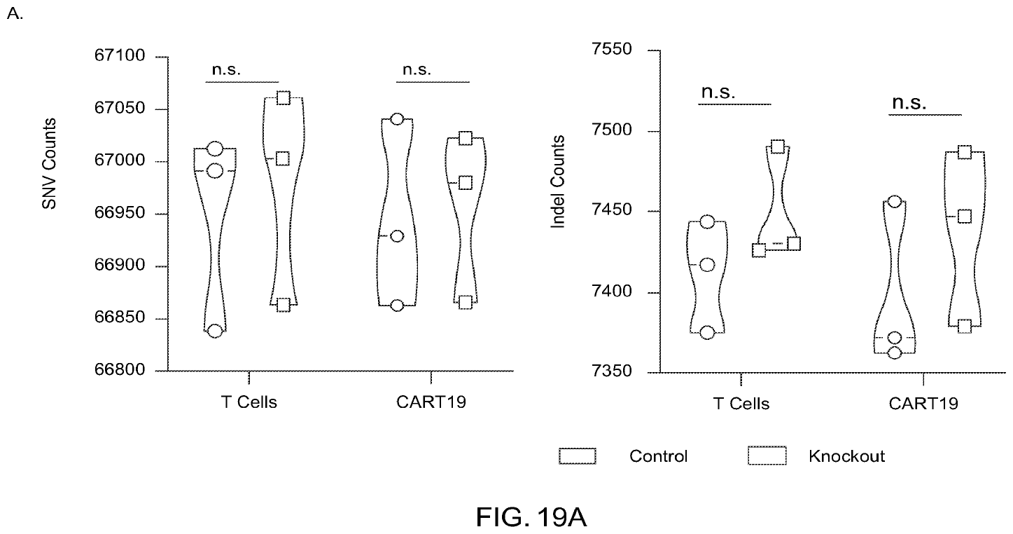

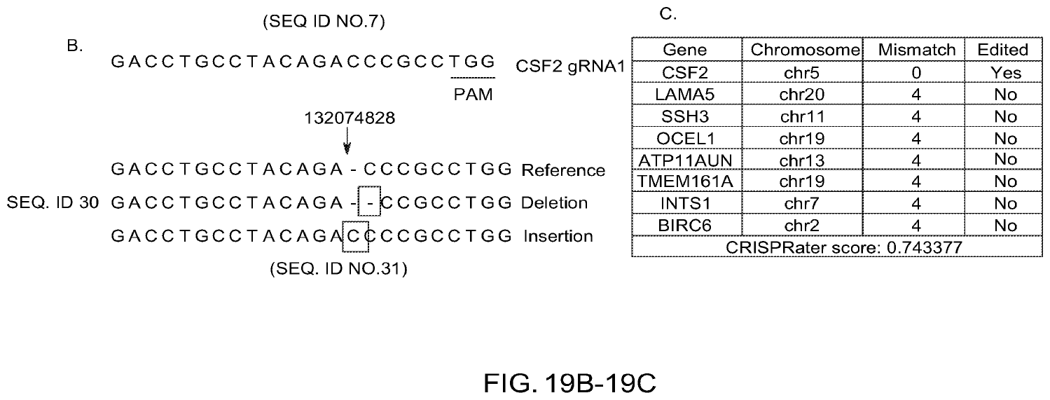

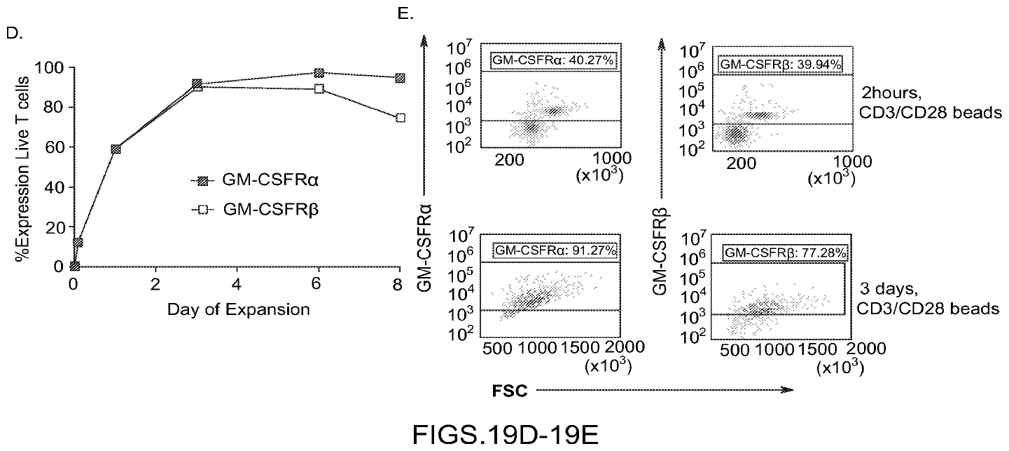

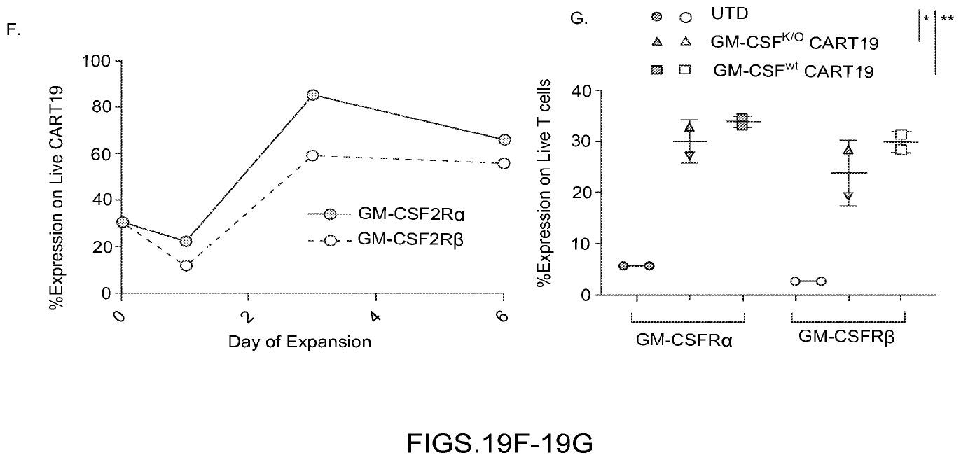

GM-CSF editing of CART19 cells is precise and efficient. Having shown that GM-CSF^{k/o} CART19 cells exhibit enhanced antigen specific proliferation, the aim was to rule out an off-target effect of the CSF2 directed gRNA. Whole exome sequencing of CART19 and GM-CSF^{k/o} CART19 cells were performed. Using CRISPR/Cas9 to disrupt CSF2 resulted in an efficiency of 60-70% (FIG. 9). Whole exome sequencing (WES) of the modified cells showed no significant difference in SNV or indels between GM-CSF^{k/o} and control (GM-CSF^{wt}) CART19 cells (FIG. 19A). WES was only significant for alterations in the intended gene target (FIGS. 19B-19C). The high precision and specificity of targeting GM-CSF exon 3 indicated that improved CART function is unlikely due to an off-target effect of the guide RNA, suggesting a direct effect of GM-CSF depletion on CART cells.

Activated T cells and CART19 cells express high levels of GM-CSF receptors. In order to determine if GM-CSF directly interacts with GM-CSF producing CART cells, whether CART cells express GM-CSF receptors (GM-CSFR) was first studied. While resting CART19 cells do not express any GM-CSF receptors, the experiments indicated that activated CART cells significantly upregulate both GM-CSF receptor (GM-CSFR) α and β subunits. This finding was significant when T cells or CART19 cells were non-specifically activated through their T cell receptors (FIGS. 19D-19F). Additionally, both GM-CSF^{k/o} and GM-CSF^{wt} CART19 cells upregulated GM-CSFRα and GM-CSFRβ when activated through the CAR with irradiated CD19+ NALM6 cells (FIG. 19G).

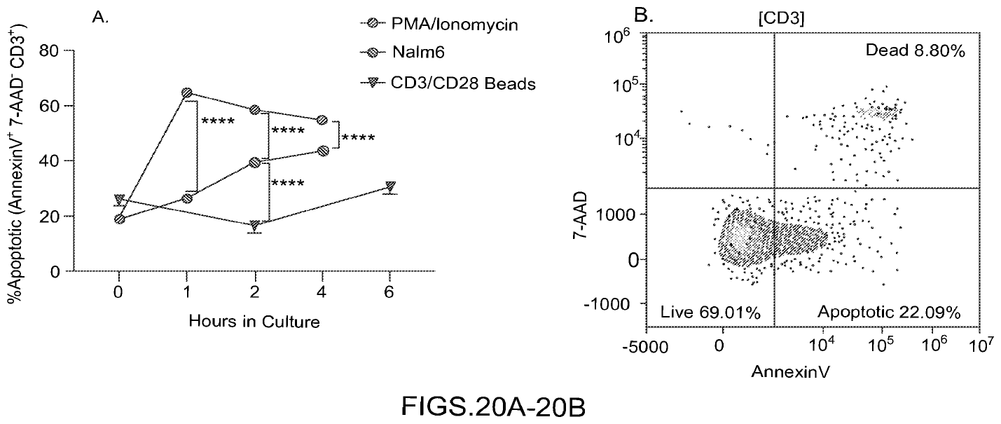

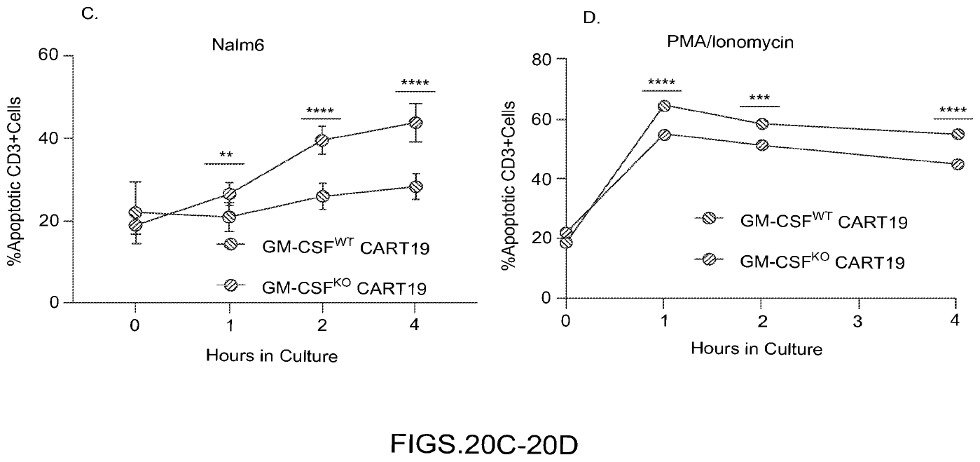

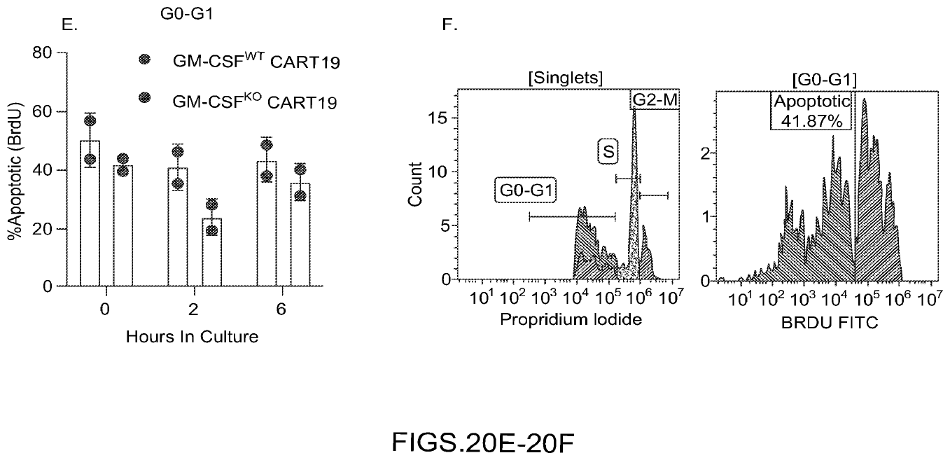

GM-CSF knockout in CART cells ameliorate CART cell apoptosis. Next, the aim was to determine if CART cells undergo apoptosis and whether GM-CSF disruption ameliorates CART cell apoptosis. The expression of Annexin V and 7-AAD was first measured by flow cytometry at early activation time points following either their stimulation through the CAR (through a co-culture with irradiated CD19+ NALM6 cells), non-specific stimulation through the TCR (CD3/CD28 beads), or non-specific stimulation with PMA/ionomycin. There were significantly more apoptotic CART19 cells (AnnexinV±7-AAD⁻) when stimulated via the CAR or PMA/ionomycin compared to non-specific stimulation through the TCR (FIGS. 20A-20B). GM-CSF^{k/o} CART19 cells exhibited significantly less apoptosis upon antigen specific stimulation compared to GM-CSF^{wt} CART19 at early time points following antigen activation (FIG. 20C). GM-CSF^{k/o} CART cells also exhibited less apoptosis compared to GM-CSF^{wt} CART cells upon PMA/ionomycin stimulation (FIG. 20D). To further validate these findings, the TUNEL assay was performed, which preferentially incorporates BrdU into apoptotic cells. The expression of BrdU on the T cells in the G0-G1 phase was measured and again found fewer apoptotic CART cells upon antigen-specific stimulation of GM-CSF^{k/o} CART19 compared to GM-CSF^{wt} CART19 cells (FIGS. 20E-20F).

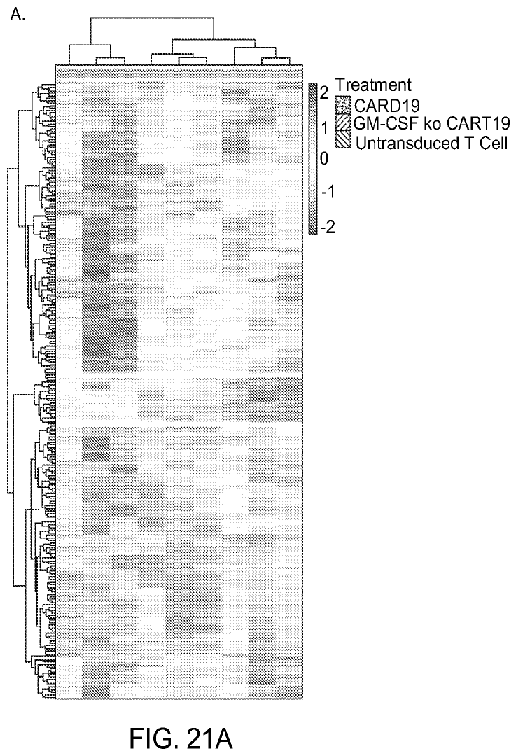

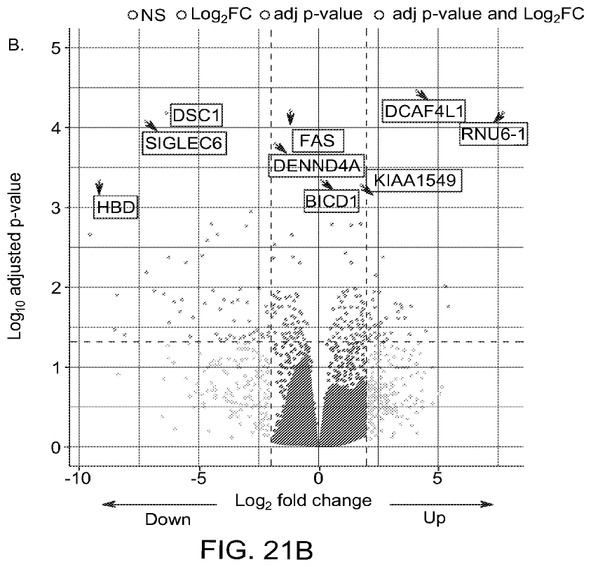

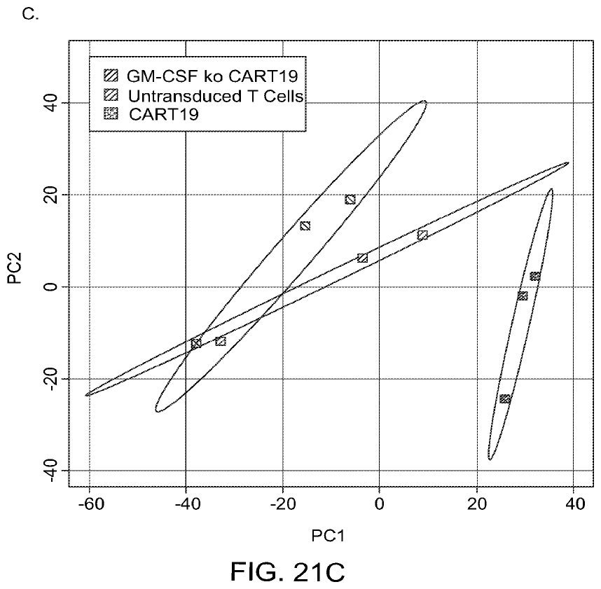

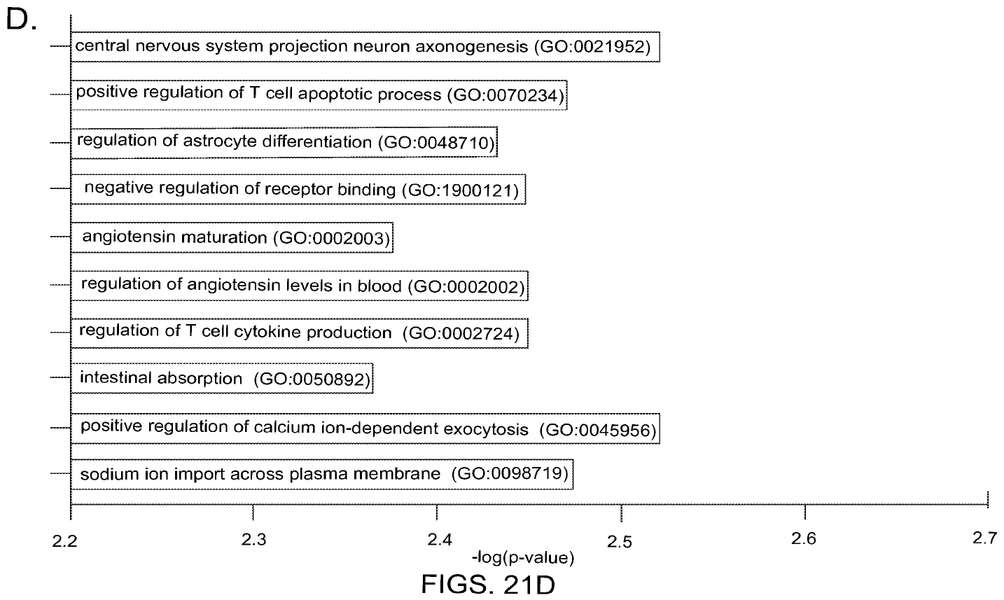

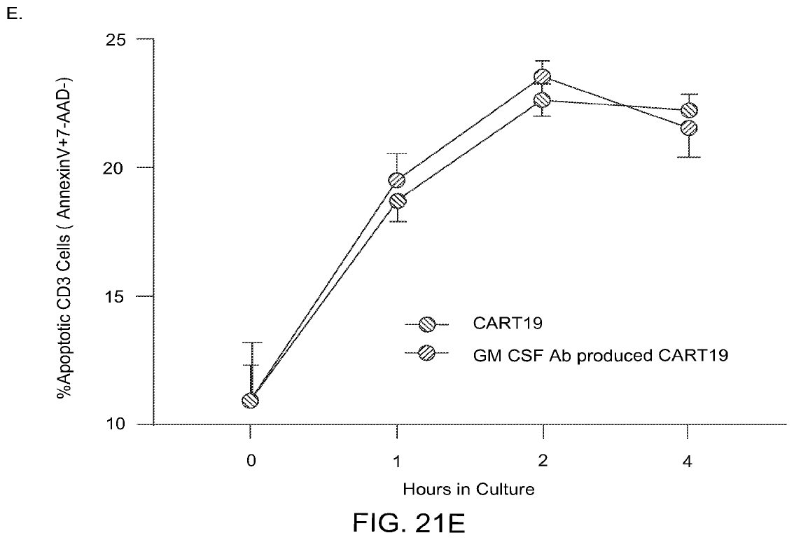

GM-CSF producing CART19 cells are intrinsically more susceptible to apoptosis. Since the data indicate that GM-CSF disruption in CART19 cells ameliorate their apoptosis upon antigen specific stimulation, and that activated CART19 cells upregulate GM-CSF receptors, the aim was to investigate the mechanisms of this effect. First, the susceptibility to apoptosis of GM-CSF^{k/o} CART cells compared to GM-CSF^{wt} CART cells was investigated by studying activation pathways in resting CART cells (at the end of CART cell manufacturing (see Methods). Transcriptome interrogation of resting GM-CSF^{k/o} CART19 revealed a distinct gene expression signature compared to resting GM-CSF^{wt} CART19 (FIG. 21A). There are more genes significantly downregulated in the GM-CSF^{k/o} CART19 compared to GM-CSF^{wt} CART19 (FIGS. 21B-21C). The transcriptome pattern of GM-CSF k/o CART19 more closely resembled untransduced T cells for wt CART19. Gene set enrichment analysis demonstrated that apoptotic pathways were most significantly altered after GM-CSF disruption in CART19 cells (FIG. 21D). These results indicated that GM-CSF^{k/o} CARTs are less susceptible to apoptosis at their baseline, independent of subsequent activation. Next, the aim was to determine whether the reduced apoptosis in GM-CSF^{k/o} CART19 cells is a direct result of GM-CSF disruption or whether it is due to interactions between secreted GM-CSF and upregulated GM-CSF receptors on CART19 cells. To test this, CART19 cells were expanded in the presence of the GM-CSF neutralizing antibody lenzilumab (see FIG. 24C Schema of CART19 production in the presence of GM-CSF blocking antibody, [FIG. 21E and FIG. 24C). Following generation of CART19 cells in the presence of GM-CSF neutralizing antibody, their apoptosis was measured after antigen specific stimulation. There was no difference in apoptotic cells (Annexin⁺ 7AAD⁻) between CART19 generated in the presence of lenzilumab or antibody controls (FIG. 21E), indicating that amelioration of apoptosis is not due to interaction between GM-CSF and its upregulated receptors on activated CART19 cells.

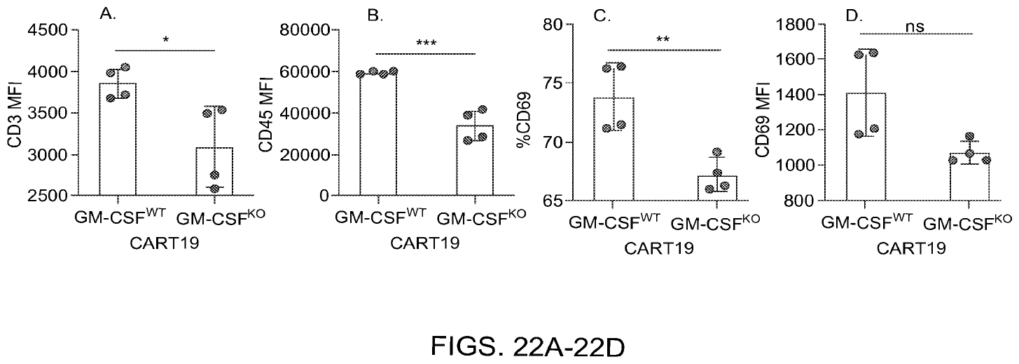

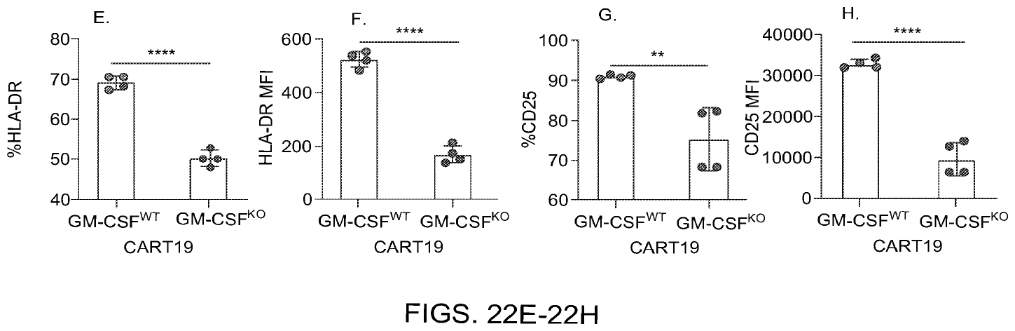

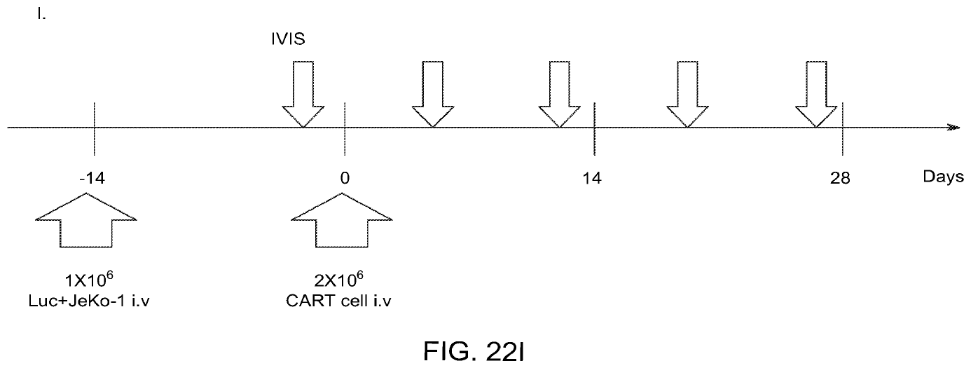

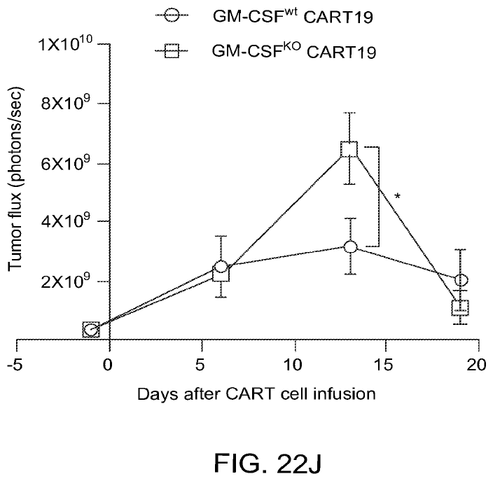

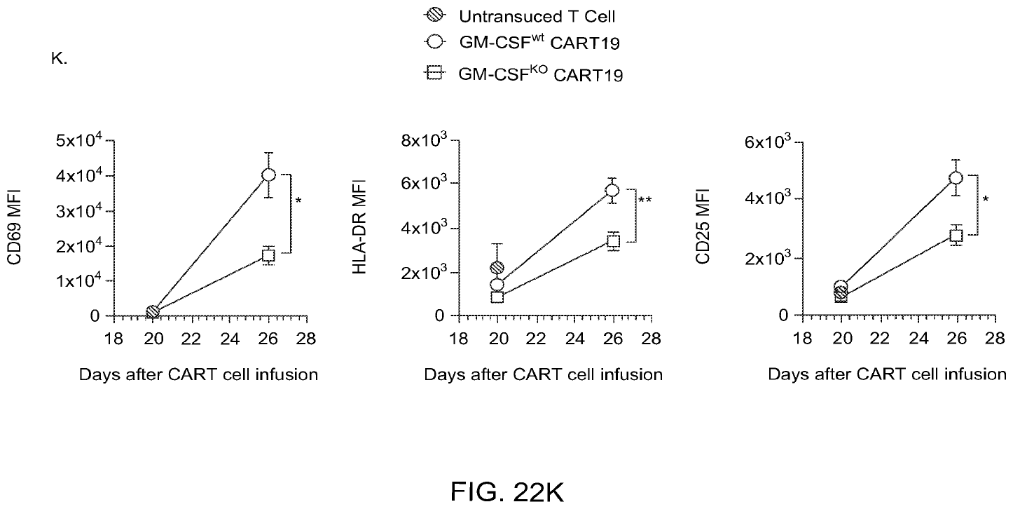

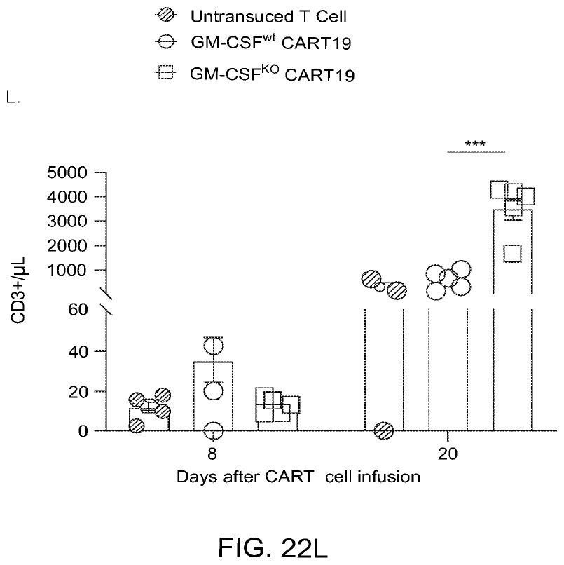

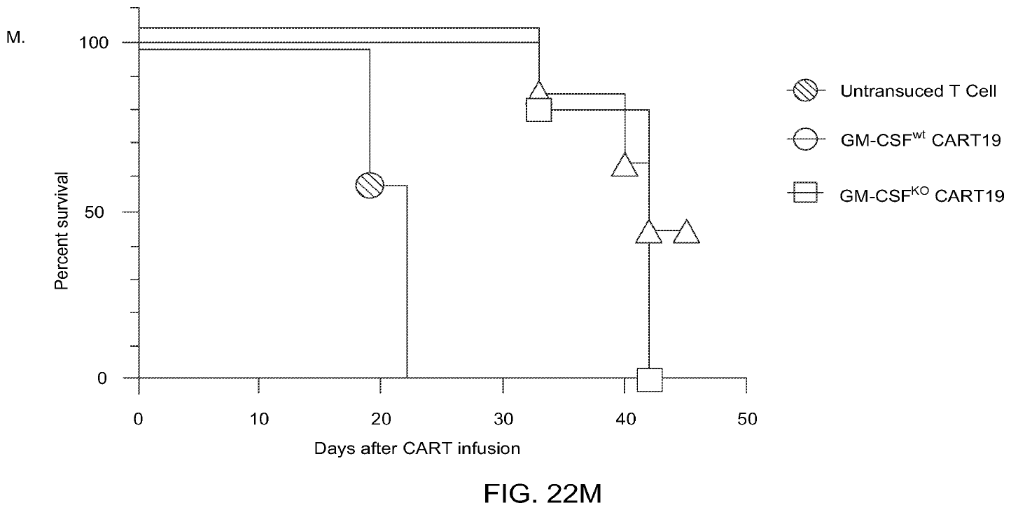

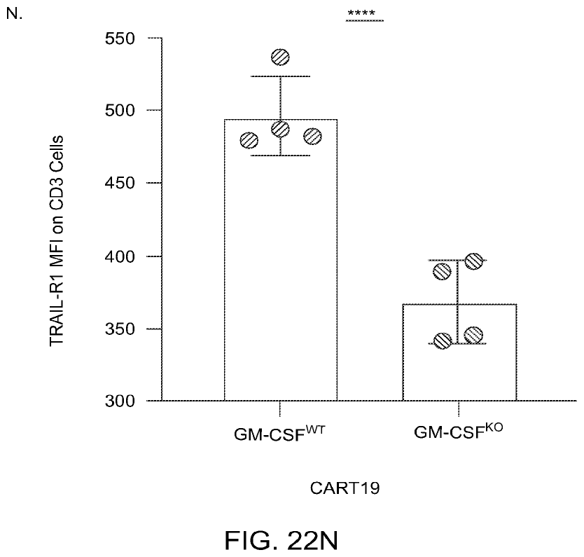

GM-CSF disruption of CART19 cells primes their activation and anti-tumor effect. Having demonstrated that GM-CSF^{k/o} CART19 cells exhibit less apoptosis upon antigen specific stimulation, the next aim was to study how GM-CSF disruption impacts the level of CART cell activation and how this impact their proliferation and antitumor activity. Twenty-four hours following antigen specific stimulation, GM-CSF^{k/o} CART19 expressed lower levels of CD3, CD45, CD69, HLA-DR, and CD25 (FIGS. 22A-22H), compared to GM-CSF^{wt} CART19 indicating reduced levels of T cells activation. Then a xenograft model for relapsed lymphoma (FIG. 22I) was used to study the impact of GM-CSF knockout of CART19 in vivo. Here, NSG mice were engrafted with JeKo-1 and randomized to receive control T cells, GM-CSF^{k/o} or GM-CSFW t CART19. GM-CSF^{k/o} CART19 cells exhibited reduced activation, but enhanced delayed proliferation, and improved antitumor activity after 13 days of treatment and show improved percent survival at 40 days compared to control T cells similat to that of GM-CSF^{wt} CART19 (FIGS. 22J-22M).

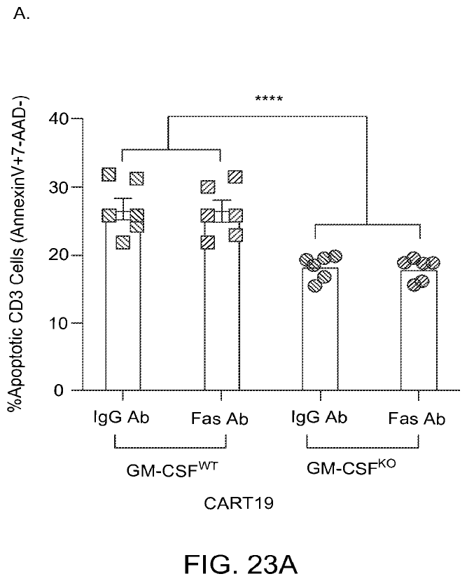

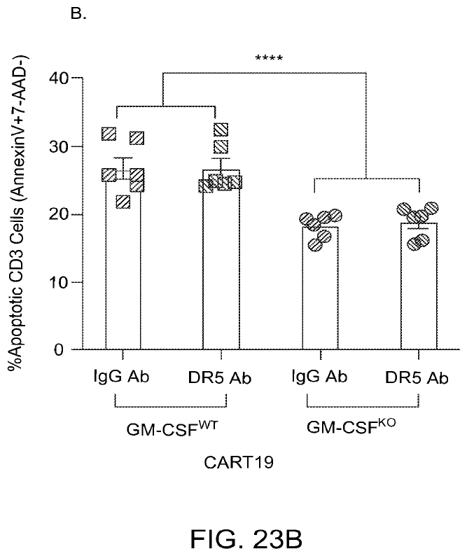

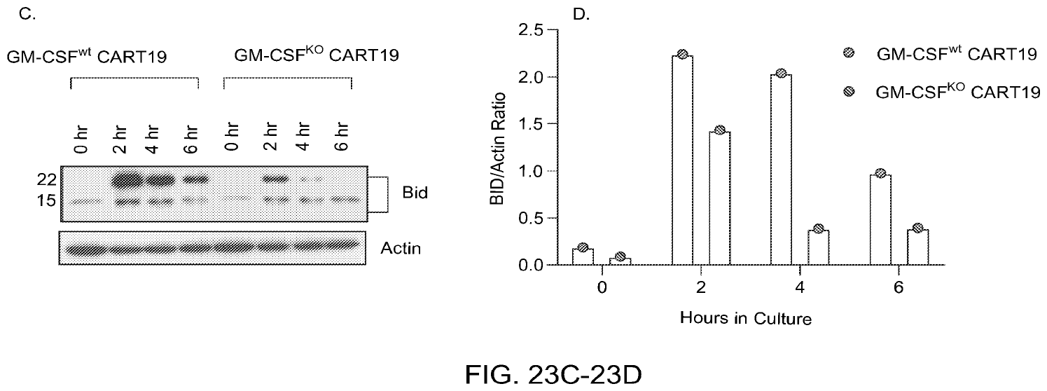

Finally, to determine the mechanisms of reduced AICD following GM-CSF disruption, the intrinsic and extrinsic regulators of apoptosis were interrogated. There was no change in GM-CSF^{k/o} CART cell apoptosis following blockade of extrinsic death pathways using TRAIL:TRAILR or Fas:FasL blockade (FIGS. 23A and 23B); however, there was a consistent reduction in Bid following GM-CSF disruption (FIGS. 23C-23D).

### Discussion

In this Example a novel mechanism by which GM-CSF affects CART cell function is described. It was identified for the first time that activated CART cells upregulate GM-CSF receptors, and that GM-CSF disruption in CART cells ameliorates their apoptosis and AICD, which in turns enhances their antitumor activity. It was demonstrates that GM-CSF mediated CART cell apoptosis is likely a result of cross talk between GM-CSF and intrinsic pathways of apoptosis.

Preclinical studies and correlative science from CART19 clinical trials have shown that inhibitory myeloid cells and cytokines are major players in both inducing CART cell toxicity and limiting anti-tumor effects. While efforts are predominantly focused on mechanisms of myeloid cell polarization and interactions with CART cells, no direct interaction between myeloid-derived cytokines and CART cells has yet been identified. In this study, a novel mechanism is reported by which GM-CSF directly affects CART cell function by promoting their apoptosis and AICD.

The present findings have significant therapeutic implications. It has become increasingly evident that CART cells are susceptible to apoptosis and AICD. CART cells upregulate Fas and its ligand (FasL), TRAIL, and TRAIL-R and are prone to FAS- and TRAIL-mediated death when a threshold of cell activation is reached. The interaction between Fas-FasL within the CART cells and tumor microenvironment limits both their persistence and anti-tumor efficacy, and genetic engineering of the CAR to include a Fas dominant negative receptor enhanced anti-tumor activity and persistence in solid tumor models. Most significantly, a recent study showed that TRAIL-deficient CART19 completely lose their antigen-specific killing abilities. As reports continue to demonstrate clinical safety and feasibility of CRISPR-modified CART cells, the present study validates CRISPR/Cas9 GM-CSF^{k/o} CART19 as a potential next generation strategy to be tested in B cell malignancies. The data presented in this this Example suggest a significant advantage for using genetic knock out of GM-CSF, which results in amelioration of apoptosis and AICD, in addition to reduction of GM-CSF levels, monocyte activation, and CART cell toxicities.

In conclusion, the present study reveals that GM-CSF producing CART19 cells are more prone to apoptosis and that GM-CSF gene expression disruption of CART19 cells reduces their apoptosis and AICD and enhances their proliferation and antitumor effect. These findings uncover a new mechanism of reduction in efficacy of CART cell therapy and importantly illuminates a new avenue to overcome CART cell apoptosis through the disruption of GM-CSF gene or GM-CSF production.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating or preventing CAR-T cell related toxicity in a subject in need thereof, the method comprising administering to the subject CAR-T cells having a GM-CSF gene inactivation, GM-CSF gene knock-down or gene knockout (GM-CSF^{k/o} CAR-T cells).

2. The method of claim 1, wherein the CAR-T cell related toxicity comprises neurotoxicity, cytokine release syndrome (CRS) or a combination thereof.

3. The method of claim 1, wherein the subject has a cancer and/or a tumor.

4. The method of claim 3, wherein the cancer is lymphoma or a leukemia.

5. The method of claim 4, wherein the lymphoma is a diffuse large B cell lymphoma (DLBCL), mantle cell lymphoma, or follicular lymphoma.

6. The method of claim 4, wherein the leukemia is acute lymphoblastic leukemia (ALL).

7. The method of claim 3, wherein the cancer is multiple myeloma.

8. The method of claim 1, wherein the GM-CSF^{k/o} CAR-T cells target tumor antigen CD19 on lymphoma or leukemia cancer

9. The method of claim 1, wherein the GM-CSF^{k/o} CAR-T cells target tumor antigen BCMA on multiple myeloma cells.

10. The method of claim 3, wherein levels of the CAR-T cells having a GM-CSF gene inactivation, GM-CSF gene knock-down or gene knockout (GM-CSF^{k/o} CAR-T cells) expand and persist in blood of the subject from a peak level of GM-CSF^{k/o} CAR-T cell expansion during the first 30 days after administration of the GM-CSF^{k/o} CAR-T cells and expansion of the GM-CSF^{k/o} CAR-T cells up to at least 90 days to 180 days after the administration of the GM-CSF^{k/o} CAR-T cells.

11. The method of claim 10, wherein GM-CSF^{k/o} CAR-T cell expansion and persistence in the blood of the subject continues for up to 24 months after administration of the GM-CSF^{k/o} CAR-T cells.

12. The method of claim 10, wherein GM-CSF^{k/o} CAR-T cell expansion and persistence in the blood of the subject achieves an anti-cancer or anti-tumor efficacy from 90 days to 24 months after administration of the GM-CSF^{k/o} CAR-T cells.

13. The method of claim 10, wherein GM-CSF^{k/o} CAR-T cells peak expansion is enhanced relative to wild type (wt) CAR-T cells and persistence as measured by CAR area under the curve (AUC) is improved relative to wt CAR-T cells.

14. The method of claim 13, wherein improved GM-CSF^{k/o} CAR-T cell expansion and persistence results in improved objective response rates (ORR), improved progression free survival (PFS), or improved overall survival (OS) compared to wt CAR-T cells.

15. The method of claim 12, wherein the anti-cancer or anti-tumor efficacy in the subject is a complete or partial remission of the cancer and/or the tumor.

16. The method of claim 12, wherein the anti-cancer or anti-tumor efficacy in the subject is a reduction or an absence of signs and symptoms of the cancer and/or the tumor.

17. A method for increasing CAR-T cell proliferation in a subject treated with GM-CSF-inactivated or GM-CSF^{k/o} CAR-T cells, the method comprising administering to the subject CAR-T cells having a GM-CSF gene inactivation, GM-CSF gene knock-down or gene knockout (GM-CSF^{k/o} CAR-T cells), wherein administration of the GM-CSF^{k/o} CAR-T cells increases CAR-T proliferation in the subject.

18. The method of claim 17, wherein administration of the GM-CSF^{k/o} CAR-T cells and expansion of the GM-CSF^{k/o} CAR-T cells reduces production of GM-CSF by 75%-99% or eliminates production of GM-CSF by the GM-CSF^{k/o} CAR-T cells.

19. The method of claim 18, wherein reduction or elimination of the production of GM-CSF by the GM-CSF^{k/o} CAR-T cells increases production and expansion of the GM-CSF by the GM-CSF^{k/o} CAR-T cells.

20. The method of claim 19, wherein increased production and expansion of the GM-CSF by the GM-CSF^{k/o} CAR-T cells reduces of eliminates CAR-T cell related toxicity in the subject, wherein the CAR-T cell related toxicity comprises neurotoxicity, cytokine release syndrome (CRS) or a combination thereof.

21. The method of claim 17, wherein the subject has a cancer and/or a tumor.

22. The method of claim 21, wherein the cancer is lymphoma or a leukemia.

23. The method of claim 22, wherein the lymphoma is a diffuse large B cell lymphoma (DLBCL).

24. The method of claim 22, wherein the leukemia is acute lymphoblastic leukemia (ALL).

25. The method of claim 21, wherein the cancer is multiple myeloma.

26. The method of claim 17, wherein the GM-CSF^{k/o} CAR-T cells target tumor antigen CD19 on lymphoma or leukemia cancer cells.

27. The method of claim 17, wherein the GM-CSF^{k/o} CAR-T cells target tumor antigen BCMA on multiple myeloma cells.

28. The method of claim 18, wherein levels of the CAR-T cells having a GM-CSF gene inactivation, GM-CSF gene knock-down or gene knockout (GM-CSF^{k/o} CAR-T cells) expand and persist in blood of the subject from a peak level of GM-CSF^{k/o} CAR-T cell expansion during the first 30 days after administration of the GM-CSF^{k/o} CAR-T cells and expansion of the GM-CSF^{k/o} CAR-T cells up to at least 90 days to 180 days after the administration of the GM-CSF^{k/o} CAR-T cells.

29. The method of claim 28, wherein GM-CSF^{k/o} CAR-T cell expansion and persistence in the blood of the subject continues for up to 24 months after administration of the GM-CSF^{k/o} CAR-T cells.

30. The method of claim 28, wherein GM-CSF^{k/o} CAR-T cell expansion and persistence in the blood of the subject achieves an anti-cancer or anti-tumor efficacy from 90 days to 24 months after administration of the GM-CSF^{k/o} CAR-T cells.

31. The method of claim 28, wherein GM-CSF^{k/o} CAR-T cells peak expansion is enhanced relative to wt CAR-T cells and persistence as measured by CAR area under the curve (AUC) is improved relative to wt CAR-T cells.

32. The method of claim 31, wherein improved GM-CSF^{k/o} CAR-T cell expansion and persistence results in improved objective response rates (ORR), improved progression free survival (PFS), or improved overall survival (OS) compared to wt CAR-T cells.

33. The method of claim 30, wherein the anti-cancer or anti-tumor efficacy in the subject is a complete or partial remission of the cancer and/or the tumor.

34. The method of claim 30, wherein the anti-cancer or anti-tumor efficacy in the subject is a reduction or an absence of signs and symptoms of the cancer and/or the tumor.


SEQUENCE LISTING (32 sequences)

SEQ ID NO: 1 - DNA, 20 bp, Artificial Sequence
  guideRNA targeting a GM-CSF nucleic acid
gacctgcctacagacccgcc

SEQ ID NO: 2 - DNA, 1458 bp, Artificial Sequence
  nucleic acid encoding a chimeric antigen receptor targeting CD19 (CAR19)
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 3 - DNA, 20 bp, Artificial Sequence
  guideRNA targeting a GM-CSF nucleic acid
tcaggagacgccgggcctcc

SEQ ID NO: 4 - DNA, 20 bp, Artificial Sequence
  guideRNA targeting a GM-CSF nucleic acid
cagcagcagtgtctctactc

SEQ ID NO: 5 - DNA, 20 bp, Artificial Sequence
  guideRNA targeting a GM-CSF nucleic acid
ctcagaaatgtttgacctcc

SEQ ID NO: 6 - DNA, 20 bp, Artificial Sequence
  guideRNA targeting a GM-CSF nucleic acid
ggccggtctcactcctggac

SEQ ID NO: 7 - DNA, 23 bp, Artificial Sequence
  guideRNA targeting a CSF2 gene nucleic acid
gacctgcctacagacccgcctgg

SEQ ID NO: 8 - DNA, 19 bp, Artificial Sequence
  CSF2 nucleic acid having a deletion caused by a CRISPR/Cas system
gacctgcctacagaccgcc

SEQ ID NO: 9 - DNA, 21 bp, Artificial Sequence
  CSF2 nucleic acid having an insertion caused by a CRISPR/Cas system
gacctgcctacagaccccgcc

SEQ ID NO: 10 - DNA, 20 bp, Artificial Sequence
  guideRNA targeting a GM-CSF nucleic acid
gcagtgctgcttgtagtggc

SEQ ID NO: 11 - DNA, 26 bp, Artificial Sequence
  guideRNA targeting a CSF2 gene nucleic acid
ccgacctgcctacagacccgcctgga

SEQ ID NO: 12 - DNA, 20 bp, Artificial Sequence
  synthetic primer
tgactacagagaggcacaga

SEQ ID NO: 13 - DNA, 21 bp, Artificial Sequence
  synthetic primer
tcacctctgacctcattaacc

SEQ ID NO: 14 - DNA, 54 bp, Homo sapiens
atgtggctgcagagcctgctgctctcgggcctcgcccagccccagcacgcagcc

SEQ ID NO: 15 - DNA, 54 bp, Homo sapiens
tacaccgacgtctcggacgacgagagcccggagcgggtcggggtcgtgcgtcgg

SEQ ID NO: 16 - DNA, 54 bp, Homo sapiens
aatgaaacagtagaagtcatctcagaaatggaagtcatctcagaaatgtttgac

SEQ ID NO: 17 - DNA, 54 bp, Homo sapiens
ttactttgtcatcttcagtagagtctttaccttcagtagagtctttacaaactg

SEQ ID NO: 18 - DNA, 55 bp, Artificial Sequence
  design Exon 3 human GM-CSF
gagccgacctgcctacagacccgcctggaggcctacagacccgcctggagctgta

SEQ ID NO: 19 - DNA, 55 bp, Artificial Sequence
  GM-CSF Exon 3 complementary strand
ctcggctggacggatgtctgggcggacctccggatgtctgggcggacctcgacat

SEQ ID NO: 20 - DNA, 55 bp, Homo sapiens
gaaacttcctgtgcaacccagattatcacctgcaacccagattatcacctttgaa

SEQ ID NO: 21 - DNA, 55 bp, Homo sapiens
ctttgaaggacacgttgggtctaatagtggacgttgggtctaatagtggaaactt

SEQ ID NO: 22 - DNA, 51 bp, Homo sapiens
tggctgcagagcctgctgctcttgggcactgtggcctgcagcatctctgca

SEQ ID NO: 23 - DNA, 52 bp, Homo sapiens
ttgggcactgtggcctgcagcatctctgcacccgcccgctcgcccagcccca

SEQ ID NO: 24 - DNA, 50 bp, Homo sapiens
tgtgcaacccagattatcacctttgaaagtttcaaagagaacctgaagga

SEQ ID NO: 25 - DNA, 50 bp, Homo sapiens
tcctgtgcaacccagattatcacctttgaaagtttcaaagagaacctgaa

SEQ ID NO: 26 - DNA, 51 bp, Homo sapiens
ttatcacctttgaaagtttcaaagagaacctgaaggactttctgcttgtca

SEQ ID NO: 27 - DNA, 20 bp, Artificial Sequence
  Guide RNA targeting exon 3 of GM-CSF
ccgcccagacatccgtccag

SEQ ID NO: 28 - DNA, 26 bp, Artificial Sequence
  Guide RNA
ggctggacggatgtctgggcggacct

SEQ ID NO: 29 - DNA, 20 bp, Artificial Sequence
  gRNA/Primer CSF2 CRISPR gRNA 1
gacctgcctacagacccgcc

SEQ ID NO: 30 - DNA, 22 bp, Artificial Sequence
  CSF2 gRNA1 Deletion
gacctgcctacagaccgcctgg

SEQ ID NO: 31 - DNA, 24 bp, Artificial Sequence
  CSF2 gRNA1 Insertion
gacctgcctacagaccccgcctgg

SEQ ID NO: 32 - DNA, 23 bp, Artificial Sequence
  CSF2 CRISPR gRNA1
ctggacggatgtctgggcggacc